(19) United States
(12) Patent Application Publication
(10) Pub. No.: US 2010/0305173 A1
(43) Pub. Date: Dec. 2, 2010

(54) HYDROXYETHYLAMINO SULFONAMIDE DERIVATIVES

(75) Inventors: Scott L. Harbeson, Cambridge, MA (US); Roger D. Tung, Lexington, MA (US); Julie F. Liu, Lexington, MA (US); Craig E. Masse, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: CoNCERT Pharmaceuticals, Inc., Lexington, MA (US)

(21) Appl. No.: 12/771,551

(22) Filed: Apr. 30, 2010

Related U.S. Application Data
(60) Provisional application No. 61/214,977, filed on Apr. 30, 2009.

(51) Int. Cl. A61K 31/428; C07D 493/04; A61K 31/34; A61K 31/427; A61P 31/18; A61P 33/06; A61K 31/36

(52) U.S. Cl. 514/367; 549/464; 514/470; 514/365; 549/362; 514/452; 548/159

(57) ABSTRACT

This invention relates to novel hydroxyethylamino sulfonamides, their derivatives, pharmaceutically acceptable salts thereof. This invention also provides compositions comprising a compound of this invention and the use of such compositions in methods of treating diseases and conditions that are beneficially treated by administering a compound with the ability to act as an HIV (human immunodeficiency virus) protease inhibitor.

### RELATED APPLICATION

This application claims the benefit of U.S. Provisional Application No. 61/214,977, filed on Apr. 30, 2009. The entire teachings of the above application are incorporated herein by reference.

### BACKGROUND OF THE INVENTION

AIDS or autoimmune deficiency syndrome is caused by the HIV virus. HIV destroys CD4 positive (CD4+) T cells, which are white blood cells crucial to maintaining the function of the human immune system. As the virus attacks those cells, the person infected with HIV is less equipped to fight off infection and disease ultimately resulting in the development of AIDS. Despite the fact that newer treatments have cut the AIDS death rate significantly, it continues to be a serious disease. By the end of 2006, the Centers for Disease Control and Prevention (CDC) estimated that over 1.1 million people in the United States were infected with the HIV virus.

When HIV infects a CD4 cell in a person's body, it copies its own genetic code into the cell's DNA. As a result, the CD4 cell becomes programmed to make new HIV genetic material and HIV proteins. These proteins are cleaved by an HIV protease to make functional new HIV particles. One important class of drugs that are used to treat HIV is protease inhibitors that inhibit the protease enzyme and thus prevent the cell from producing new viruses. It is recommended that a protease inhibitor be used in combination with at least two other HIV drugs to treat HIV infection.

One shortcoming in the use of the currently marketed protease inhibitors is that they are susceptible to fairly rapid metabolism in the liver by the cytochrome enzyme CYP3A4. To overcome this limitation, protease inhibitors are often co-administered with a “booster” such as the HIV protease inhibitor ritonavir that inhibits the CYP enzyme. However, ritonavir itself is associated with a number of side effects such as nausea, vomiting, diarrhea and loss of appetite. It would be advantageous therefore to have an HIV protease inhibitor that eliminates or reduces the need for ritonavir co-administration.

Darunavir, also known as Prezista™, or [(1S,2R)-3-[[(4-aminophenyl)sulfonyl](2-methylpropyl)amino]-2-hydroxy-1-(phenylmethyl)propyl]-carbamic acid (3R,3aS,6aR)-hexahydrofuro[2,3-b]furan-3-yl ester monoethanolate, is one of the new HIV protease inhibitors. First approved in the U.S. in June, 2006, it acts by selectively inhibiting the cleavage of HIV encoded Gag-Pol polyproteins in infected cells, thereby preventing the formation of mature virus particles. (See FDA label for darunavir@http://www.fda.gov/cder/foi/label/2006/021976s001lbl.pdf).

The most common adverse events experienced by patients dosed with darunavir include, but are not limited to, diarrhea, nausea, abdominal pain, constipation, headache, common cold, increased amylase, neutropenia, and nasopharyngitis. Darunavir is rapidly metabolized by cytochrome P450 3A4 (CYP3A4) and as a result requires co-dosing with ritonavir to maintain suitable plasma concentrations. Co-dosing with ritonavir contraindicates co-dosing with other drugs that are highly dependent on CYP3A4 for clearance and for which elevated plasma concentrations are associated with serious and/or life-threatening events. (See FDA label for darunavir@(http://www.fda.gov/cder/foi/label/2006/021976s001lbl.pdf).

Brecanavir, or N-[3-[N-(1,3-Benzodioxol-5-ylsulfonyl)-N-isobutylamino]-2(R)-hydroxy-1-(S)-[4-(2-methylthiazol-4-ylmethoxy)benzyl]propyl]carbamic acid (3R,3aS,6aR)-perhydrofuro[2,3-b]furan-3-yl ester, is a highly potent HIV protease inhibitor which demonstrated potent viral load suppression in patients infected with either wild-type HIV or virus containing mutations that confer resistance to other HIV protease inhibitors. (See Lalezari, J P et al., J. Antimicrob. Chemother. 2007 60: 170 (2007)).

Brecanavir proved difficult to formulate and its development was discontinued due to reportedly insurmountable issues. (See GlaxoSmithKline press release dated 18 December 2006 (http://us.gsk.com/html/media-news/pressreleases/2006/2006_{—}12_{—}18_GSK1001.htm)).

Despite the beneficial activities of darunavir and brecanavir, there is a continuing need for new compounds to treat HIV infection, especially protease inhibitors that are less reliant on ritonavir co-administration.

### SUMMARY OF THE INVENTION

This invention relates to novel hydroxyethylamino sulfonamides, their derivatives, pharmaceutically acceptable salts thereof. This invention also provides compositions comprising a compound of this invention and the use of such compositions in methods of treating diseases and conditions that are beneficially treated by administering a compound with the ability to act as an HIV (human immunodeficiency virus) protease inhibitor.

### BRIEF DESCRIPTION OF THE DRAWINGS

FIG. 1 depicts a plot of percentage of starting material of various compounds of the invention over time of incubation with human liver microsomes.

FIG. 2 depicts a plot of percentage of starting material of various compounds of the invention over time of incubation with human liver microsomes.

FIG. 3 depicts a plot of percentage of starting material of various compounds of the invention over time of incubation with human liver microsomes.

### DETAILED DESCRIPTION OF THE INVENTION

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of an HIV protease inhibitor such as darunavir will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; Gannes L Z et al., Comp Biochem Physiol Mol Integr Physiol, 1998, 119: 725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3340 (50.1% deuterium incorporation) at each atom designated as deuterium in said compound.

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopologue” refers to a species that differs from a specific compound of this invention only in the isotopic composition thereof.

The term “compound,” as used herein, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers. Methods of obtaining or synthesizing an individual enantiomer for a given compound are known in the art and may be applied as practicable to final compounds or to starting material or intermediates.

Unless otherwise indicated, when a disclosed compound has one or more chiral centers and is named or depicted by a structure without specifying the stereochemistry at one or more of those stereocenters, it is understood to represent compounds bearing all possible stereoisomers of the unspecified chiral centers.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert”, “^{t}”, and “t-” each refer to tertiary. “US” refers to the United States of America.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

The term “optionally substituted” refers to the optional replacement of one or more hydrogen atoms with another moiety.

The term “halo” refers to any of —Cl, —F, —Br, or —I.

The term “carboxy” refers to —C(O)OH

The term “oxo” refers to ═O.

The term “alkoxy” refers to —O-alkyl.

The term “alkylamino” refers to —NH-alkyl.

The term “dialkylamino” refers to N(alkyl)-alkyl, wherein the two alkyl moieties are the same or different.

The term “alkyl” refers to straight or branched alkyl chains of from 1 to 12 carbon atoms, unless otherwise specified. Examples of straight chained and branched alkyl groups include methyl, ethyl, n-propyl, iso-propyl, n-butyl, sec-butyl, tert-butyl, pentyl, hexyl, pentyl and octyl.

The term “alkylene” refers to straight or branched saturated divalent chains of from 1 to 12 carbon atoms, unless otherwise specified. Examples of straight chained and branched alkylene groups include —CH₂—, —CH₂—CH₂—, —CH₂—CH₂—CH₂—, —CH₂—CH(CH₃)—, —CH₂—CH₂—CH₂—CH₂—, —CH₂—CH(CH₃)—CH₂—, and —CH₂—C(CH₃)₂—CH₂—.

Examples of optional substituents on an alkyl group, such as a C₁₋₇ alkyl include halo, cyano, hydroxyl, carboxy, alkoxy, oxo, amino, alkylamino, dialkylamino, cycloheteroalkyl, aryl, and heteroaryl.

The term “cycloheteroalkyl” refers to an optionally substituted non-aromatic monocyclic, bicyclic, tricyclic, spirocyclic, or tetracyclic ring system which includes one or more heteroatoms such as nitrogen, oxygen or sulfur in at least one of the rings. Each ring can be four, five, six, seven or eight-membered. Examples include tetrahydrofuryl, tetrahydrothiophenyl, morpholino, thiomorpholino, pyrrolidinyl, piperazinyl, piperidinyl, and thiazolidinyl, along with the cyclic form of sugars. Suitable substituents on a cycloheteroalkyl can include, but are not limited to for example, alkyl, halo, cyano, hydroxyl, carboxy, alkoxy, oxo, amino, alkylamino and dialkylamino. Examples of alkyl substituted cycloheteroalkyls include 4-methylpiperazin-1-yl and 4-methylpiperidin-1-yl.

The term “aryl” refers to optionally substituted carbocyclic aromatic groups such as phenyl and naphthyl. Suitable substituents on an aryl can include, but are not limited to for example, alkyl, halo, cyano, hydroxyl, carboxy, alkoxy, amino, alkylamino and dialkylamino.

The term “heteroaryl” refers to an optionally substituted monocyclic aromatic group comprising one or more heteroatoms such as nitrogen, oxygen or sulfur in the ring, such as imidazolyl, thienyl, furyl, pyridyl, pyrimidyl, pyranyl, pyrazolyl, pyrrolyl, pyrazinyl, thiazolyl, oxazolyl, and tetrazolyl. Heteroaryl groups also include fused polycyclic aromatic ring systems in which at least one ring comprises one or more heteroatoms such as nitrogen, oxygen or sulfur. Examples include benzothienyl, benzofuryl, indolyl, quinolinyl, benzothiazole, benzoxazole, benzimidazole, quinolinyl, isoquinolinyl and isoindolyl. Suitable substituents on a heteroaryl can include, but are not limited to for example, alkyl, halo, cyano, hydroxyl, carboxy, alkoxy, amino, alkylamino and dialkylamino.

Unless otherwise specified, the term “α-amino acid (AA)” includes α-amino acids having a (D)-, (L)- or racemic (D,L) configuration. It is understood that in the case of the variable R⁸-AA α-amino acid, it is bonded to R⁸ through the carbonyl carbon which is directly bonded to the α-carbon of the amino acid. In accordance with the structure of Formula I, such a linkage results in the formation of an ester.

### Therapeutic Compounds

In one embodiment, the present invention provides a compound of Formula A:

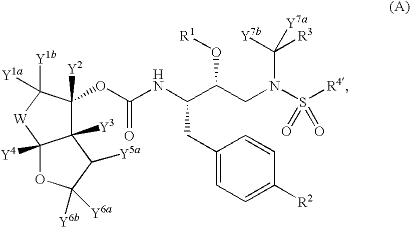

or a pharmaceutically acceptable salt thereof, wherein:

W is —O—, —CH₂—, or —CD₂-;

each Y is independently selected from hydrogen or deuterium;

R¹ is hydrogen or —(C₁-C₁₁ alkylene)-R⁶, wherein, the C₁-C₁₁ alkylene is optionally substituted by one or more groups independently selected from halo, cyano, —OH, ═O, —SH, —PO₃H, —PO₃(C₁₋₆ alkyl), ═N, —NH₂, NH(C₁-C₄ alkyl), N(C₁-C₄ alkyl)₂, —C₁-C₄ alkyl, cycloheteroalkyl, cycloheteroalkylalkyl, aryl, arylalkyl, heteroaryl, heteroarylalkyl, or a side group of a naturally occurring amino acid, and up to 4 methylene units in the C₁-C₁₁ alkylene are optionally and independently replaced with —O—, —S—, —S(O)—, S(O)₂—, —P(O)₂—, —P(O)(OH)—, —NH—, and —N(C₁-C₆ alkyl)-, provided that the terminal end of R¹ bonded to the oxygen is not oxygen or nitrogen; and

R⁶ is selected from hydrogen, —N(R⁷)(R⁷), optionally substituted C₁-C₈ alkyl, C₁-C₈ alkoxy, heteroaryl or cycloheteroalkyl wherein the heteroaryl or cycloheteroalkyl is optionally substituted with C₁-C₈ alkyl, and wherein each R⁷ is independently selected from hydrogen, C₁-C₈ alkyl, and C₁-C₈ alkoxy;

R² is selected from hydrogen, —OH and —O—Z—R^{10a};

Z is a C₁₋₄ alkylene that is optionally substituted with deuterium;

R^{10a} is selected from hydrogen, methoxy, phenyl, cyanophenyl, pyridyl, 3-cyanopyridyl, pyrazinyl, pyrazol-1-yl, 3,5-dimethylpyrazol-1-yl, isoxazol-3-yl, 5-methyl-isoxazol-3-yl, 2-methyl-thiazol-4-yl, 5-methyl-thiazol-4-yl, 2,5-dimethyl-thiazol-4-yl, 2-methyl-thiazol-5-yl, 4-methyl-thiazol-5-yl, 2,4-dimethyl-thiazol-5-yl, 2-thienyl, 4-morpholinyl, 4-methylpiperazin-1-yl, (C₁₋₄ alkyl)aminocarbonyl, trifluoromethyl, hydroxymethyl, (C₁₋₄ alkyl)sulfonylamino, (C₁₋₄ alkoxy)methylcarbonylamino, (C₁₋₄ alkyl)carbonylamino, phenylcarbonylamino, (C₁₋₄ alkoxy)carbonylamino, 2-furanylcarbonylamino, 2-thienylcarbonylamino, (C₁₋₄ alkyl)carbamyloxy, bis(methoxyethyl)amino, C₆H₅C(═N—CN)—NH—, 1H-pyrazol-5-ylamino, pyrimidin-2-ylamino, 1H-1,2,4-triazol-1-yl, thiazol-2-yl, thiazol-4-yl, thiazol-5-yl, O—[CH₂CH₂O]ₚ—CH₃, and 5-phenyl-1,2,4-oxadiazol-3-yl, wherein any methyl, alkyl or alkoxy moiety in R^{10a} is optionally substituted with one or more deuterium;

R³ is a group that is optionally substituted with one or more deuterium and is selected from phenyl, C₃-C₆ cycloalkyl, C₅-C₆ cycloalkenyl, —CH₂—CH(CH₃)₂, —CH(CH₃)₂, —CH(C₂H₅)₂, —CH(CH₃)—C₂H₅, —CH═CH₂, and —C(CH₃)₂—(CH₂)ₘ—NH—R¹¹;

R¹¹ is selected from hydrogen, —C(O)OCH₃, —C(O)CH₃, —C(O)NHCH₃ and —S(O)₂CH₃;

m is 1, 2, 3, 4 or 5;

each p is independently 1, 2, 3, 4, or 5; and

R^{4′} is a group that is optionally substituted with one or more deuterium and is selected from 2,3-dihydrobenzofuran-5-yl, 3-oxo-2,3-dihydrobenzofuran-5-yl, chromanyl-6-yl, 4-oxo-chromanyl-6-yl, 4-oxo-4H-chromenyl-6-yl, 2,3-dihydrobenzo[b][1,4]dioxin-6-yl, 3,4-dihydro-2H-benzo[b][1,4]oxazin-6-yl, 3,4-dihydro-2H-benzo[b][1,4]oxazin-7-yl, 3-oxo-3,4-dihydro-2H-benzo[b][1,4]oxazin-7-yl, 3-oxo-3,4-dihydro-2H-benzo[b][1,4]oxazin-6-yl,

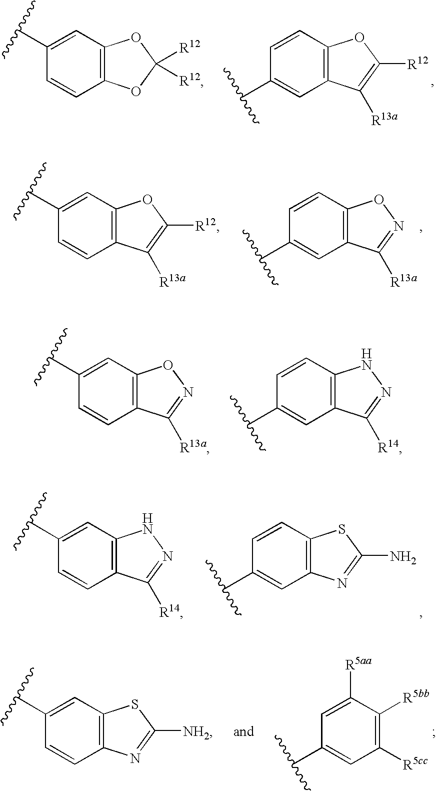

each R⁵ is independently selected from hydrogen, —F, —NH₂, —OH, —OCF₃, —CF₃, —CH₃, —CD₃, —OCH₃, —OCD₃, —CH₂—OCD₃, —CD₂-OCD₃, —CD₂-OCH₃, —CH₂OH, —CD₂OH, —OCH₂CH₃, —OCD₂CD₃, —OCH(CH₃)₂, —OCD(CH₃)₂, —OCD(CD₃)₂, O—[CH₂CH₂O]ₚ—CH₃, O—[CH₂CH₂O]ₚ—CD₃, O—[CD₂CD₂O]ₚ—CD₃, and —OCH(CD₃)₂;

each R¹² is independently selected from hydrogen, deuterium, —CH₃, and —CD₃;

R^{13a} is selected from hydrogen, deuterium, —CH₃, —CD₃, —CH₂—O—CH₃, —CH₂—O—CD₃, —CD₂-O—CD₃, —CH₂-(1-piperidinyl), —CH₂-(4-morpholinyl), —CH₂—NH—X—R¹⁵, and —CD₂-NH—X—R¹⁵;

X is selected from a bond, —C(O)—, —CO₂— and —SO₂—;

R¹⁵ is selected from C₁₋₆ alkyl, C₃₋₆ cycloalkyl, or C₄₋₁₀ (cycloalkyl)alkyl, wherein R¹⁵ is optionally substituted with one or more substituents independently selected from deuterium, —CF₃, phenyl, —CH₂-phenyl, —CD₂-phenyl, 2-furanyl, —CH₂-(2-furanyl), —CD₂-(2-furanyl), —CH₂-(2-benzofuranyl), —CD₂-(2-benzofuranyl), 2-pyridyl, 3-pyridyl, —CH₂-(2-pyridyl), —CD₂-(2-pyridyl), —CH₂-(3-pyridyl), —CD₂-(3-pyridyl), 4-thiazolyl, —CH₂-(4-thiazolyl), and —CD₂-(4-thiazolyl);

R¹⁴ is selected from hydrogen, deuterium, —CH₃, —CD₃, and —N(R¹⁶)₂;

each R¹⁶ is independently selected from C₁₋₆ alkyl, C₃₋₆ cycloalkyl, and C₄₋₁₀ (cycloalkyl)alkyl, wherein R¹⁶ is optionally substituted with one or more substituents independently selected from deuterium, halo, hydroxyl, cyano, —N(R¹⁷)₂, —C(O)—R¹⁷, —CO₂R¹⁷, —C(O)—N(R¹⁷)₂;

each R¹⁷ is independently selected from C₁₋₆ alkyl, C₃₋₆ cycloalkyl, or C₄₋₁₀ (cycloalkyl)alkyl, wherein R¹⁷ is optionally substituted with one or more deuterium; provided that when W is —O—, R² is hydrogen and R³ is isopropyl optionally substituted with one or more deuterium, then R^{4′} is other than

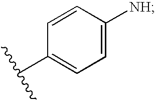

and further provided that at least one Y is deuterium or at least one of R¹, R², R³ or R^{4′} comprises a deuterium atom.

In a particular embodiment of Formula A, when W is —O—, and R³ is isopropyl optionally substituted with one or more deuterium, then R^{4′} is other than

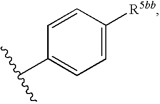

wherein R^{5bb} is —OH, NH₂ or O—[CH₂CH₂O]ₚ—CH₃, O—[CH₂CH₂O]ₚ—CD₃, O—[CD₂CD₂O]ₚ—CD₃.

In another embodiment of Formula A, R^{4′} is selected from:

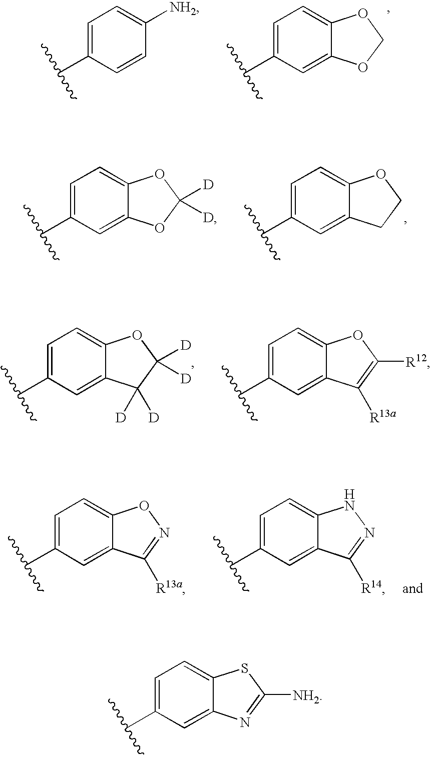

In a more specific aspect of this embodiment, W is —O—; each Y¹, Y², Y³, Y⁴, Y each Y⁵ and each Y⁶ is deuterium; R¹ is hydrogen; R³ is —CD(CD₃)₂; and R^{4′} is selected from

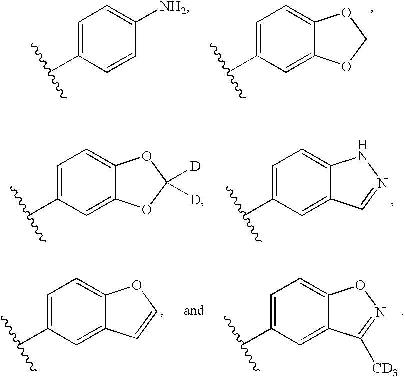

### In

another more specific aspect of this embodiment, W is —O—; each Y¹, and Y² is deuterium; Y³, Y⁴, each Y⁵ and each Y⁶ is hydrogen; R¹ is hydrogen; R³ is —CD(CD₃)₂; and R^{4′} is selected from

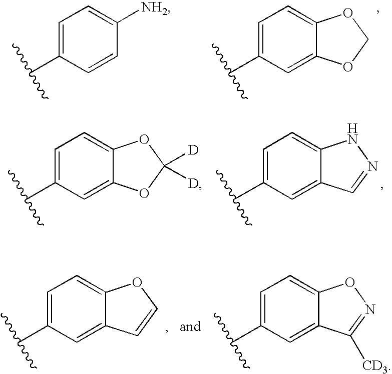

In another embodiment of Formula A, the compounds is represented by structural Formula A-1:

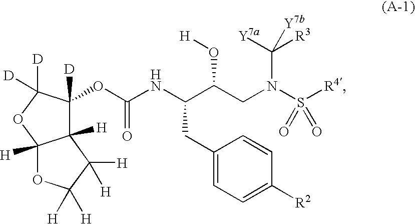

or a pharmaceutically acceptable salt thereof, wherein:

R² is selected from: hydrogen

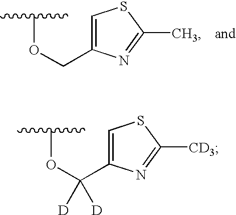

R³ is selected from: —CH₂CH(CH₃)₂, —CD₂CD(CH₃)₂, —CD₂CD(CD₃)₂, —CH(CH₃)₂, —CD(CH₃)₂, and —CD(CD₃)₂; and

R^{4′} is selected from:

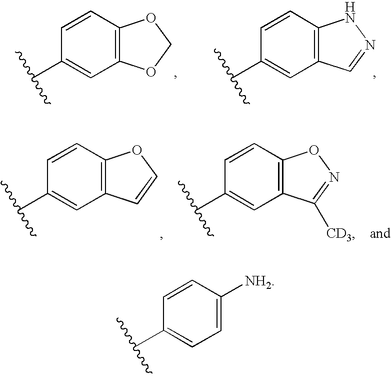

In one aspect of Formula Ia, Y^{7a} and Y^{7b} are the same. In another aspect both Y^{7a} and Y^{7b} are deuterium.

In another aspect, compounds of Formula A-1 are set forth in Table 1.

TABLE 1
Compounds of Formula A-1.

| | Each | | | |
|---|---|---|---|---|
| Compound | Y⁷ | R² | R³ | R^{4′} |
| 101 | H | H | —CD(CD₃)₂ | 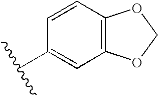 |
| 102 | D | H | —CD(CD₃)₂ | 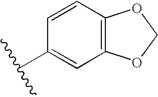 |
| 103 | H | H | —CD(CD₃)₂ | 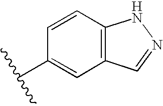 |
| 104 | D | H | —CD(CD₃)₂ | 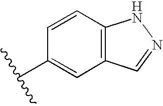 |
| 105 | H | H | —CD(CD₃)₂ | 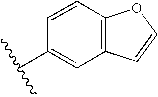 |
| 106 | D | H | —CD(CD₃)₂ | 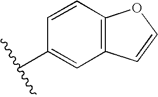 |
| 107 | H | H | —CD(CD₃)₂ | 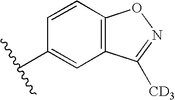 |
| 108 | D | H | —CD(CD₃)₂ | 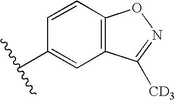 |
| 109 | H | 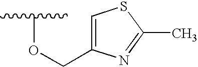 | —CD(CD₃)₂ | 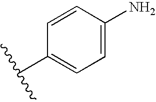 |
| 110 | D | 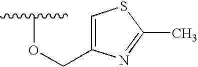 | —CD(CD₃)₂ | 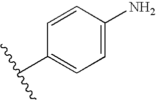 |
| 111 | H | 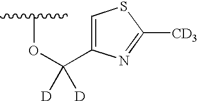 | —CD(CD₃)₂ | 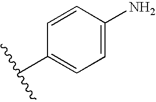 |
| 112 | D | 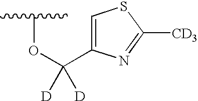 | —CD(CD₃)₂ | 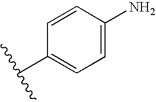 |
| 113 | H | 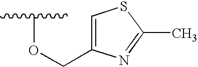 | —CD(CD₃)₂ | 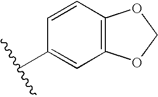 |
| 114 | D | 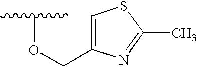 | —CD(CD₃)₂ | 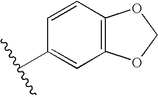 |
| 115 | H | 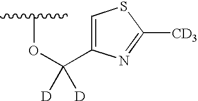 | —CD(CD₃)₂ | 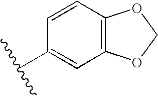 |
| 116 | D | 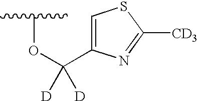 | —CD(CD₃)₂ | 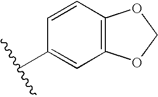 |
| 117 | H | 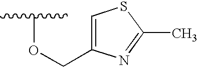 | —CD(CD₃)₂ | 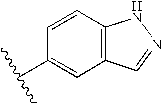 |
| 118 | D | 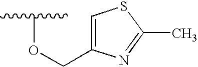 | —CD(CD₃)₂ | 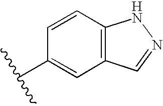 |
| 119 | H | 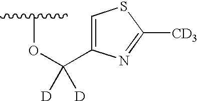 | —CD(CD₃)₂ | 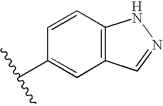 |
| 120 | D | 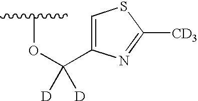 | —CD(CD₃)₂ | 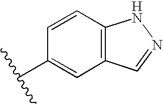 |
| 121 | H | 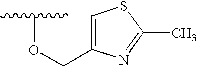 | —CD(CD₃)₂ | 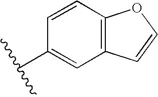 |
| 122 | D | 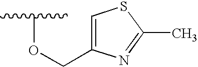 | —CD(CD₃)₂ | 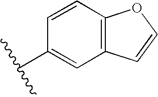 |
| 123 | H | 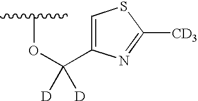 | —CD(CD₃)₂ | 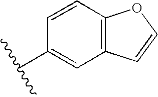 |
| 124 | D | 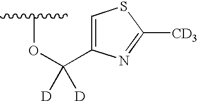 | —CD(CD₃)₂ | 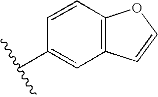 |
| 125 | H | 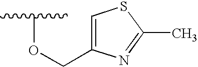 | —CD(CD₃)₂ | 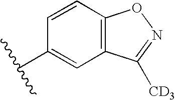 |
| 126 | D | 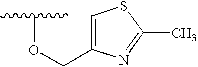 | —CD(CD₃)₂ | 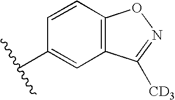 |
| 127 | H | 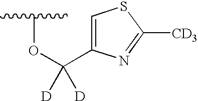 | —CD(CD₃)₂ | 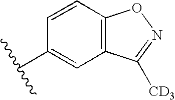 |
| 128 | D | 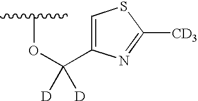 | —CD(CD₃)₂ | 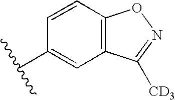 |

or a pharmaceutically acceptable salt of any of the foregoing.

More specifically, the compounds of Table 1 have the following structural formula:

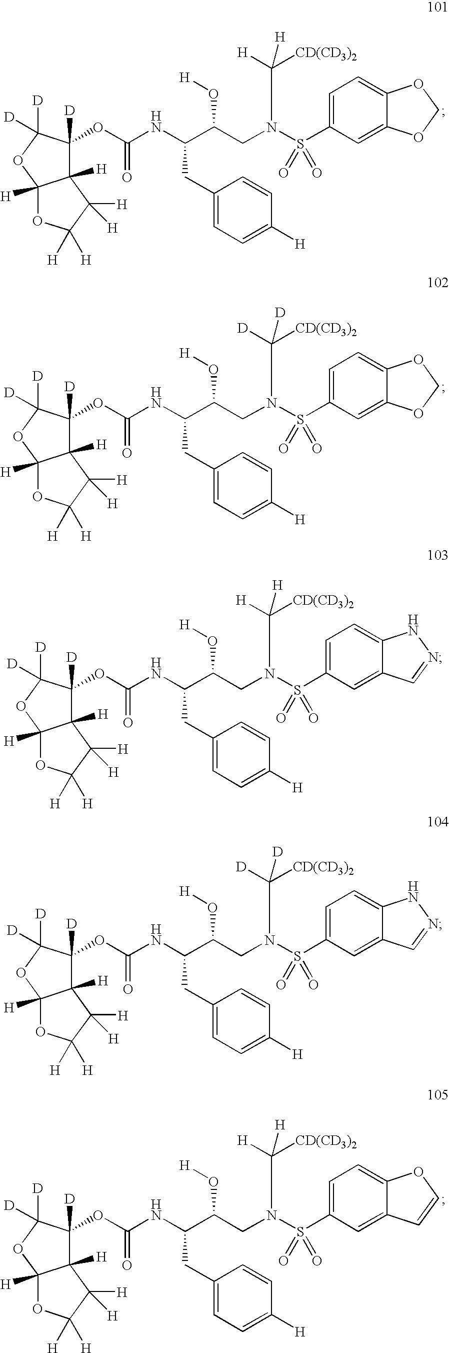

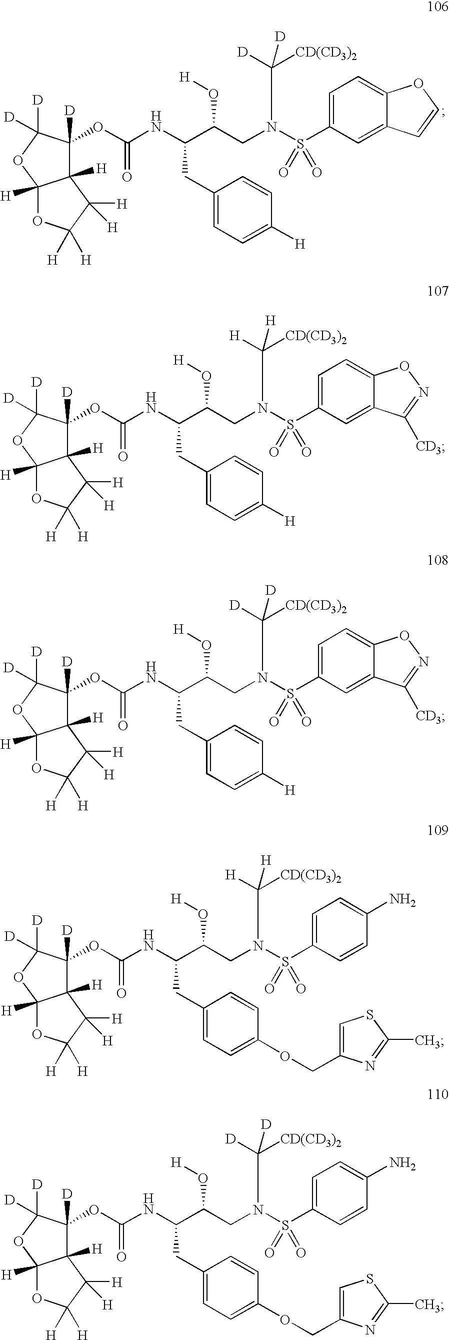

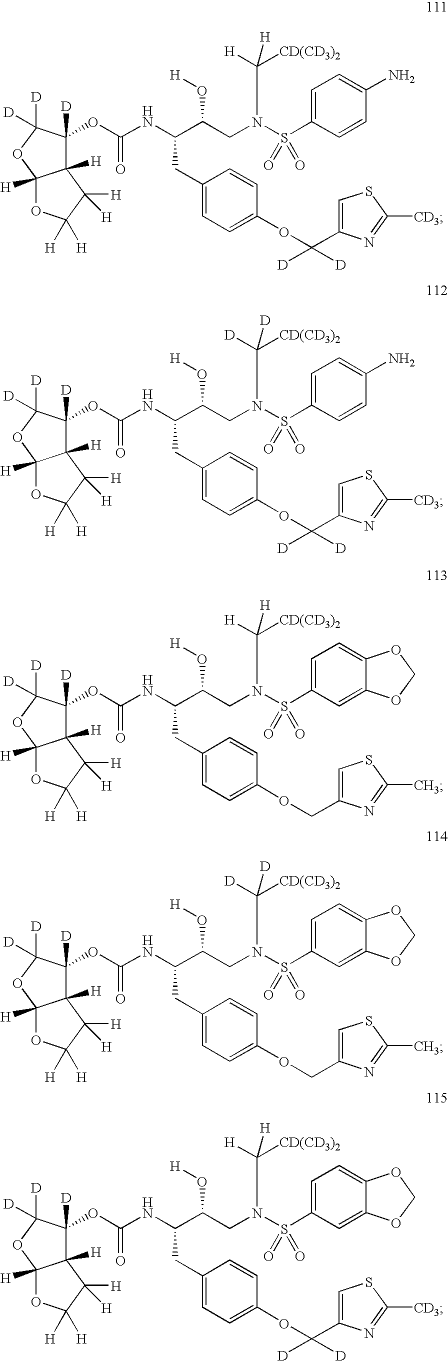

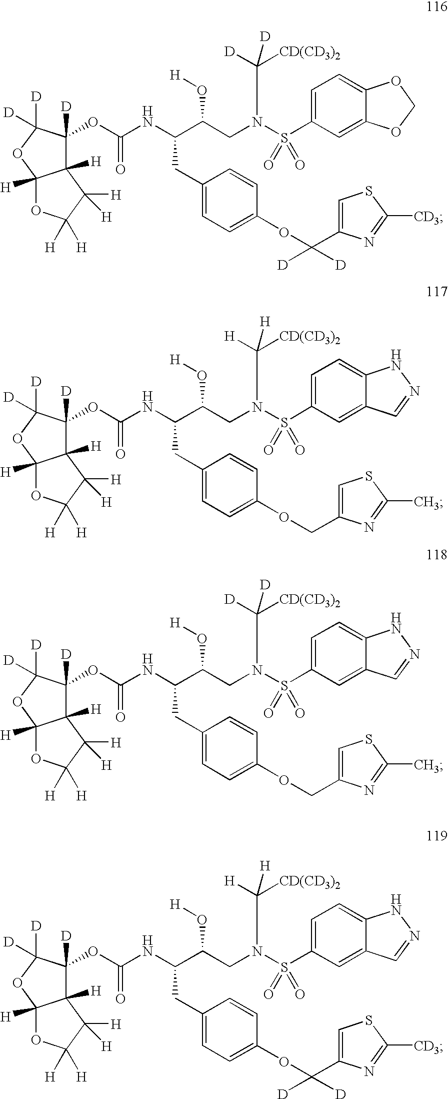

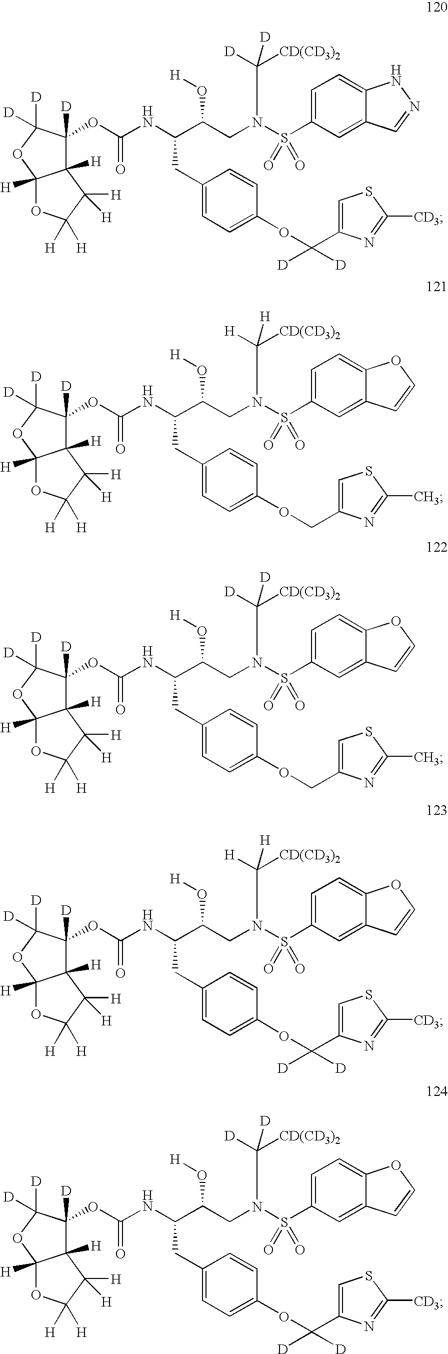

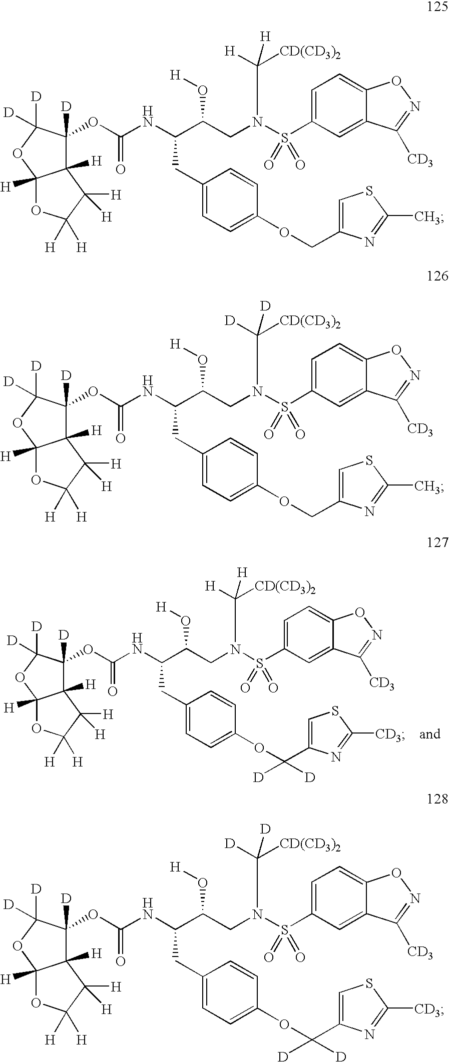

or pharmaceutically acceptable salts of any of the foregoing.

In an alternate embodiment, the present invention provides a compound of Formula I:

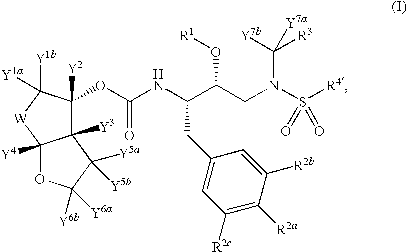

or a pharmaceutically acceptable salt thereof, wherein:

W, each Y, R¹ (including R⁶ and R⁷ portions thereof), and R³ (including the R¹¹ and m portions thereof) are as defined for Formula A;

R^{2a} is hydrogen, —OH, —F, —O—Z—R¹⁰, or C₁₋₄ alkyl optionally substituted with one or more —F, wherein Z is as defined in Formula A;

each of R^{2b} and R^{2c} is independently hydrogen or —F;

R¹⁰ is selected from hydrogen, deuterium, phenyl, cyanophenyl, pyridyl, 3-cyanopyridyl, pyrazinyl, pyrazol-1-yl, 3,5-dimethylpyrazol-1-yl, isoxazol-3-yl, 5-methyl-isoxazol-3-yl, 2-methyl-thiazol-4-yl, 5-methyl-thiazol-4-yl, 2,5-dimethyl-thiazol-4-yl, 2-methyl-thiazol-5-yl, 4-methyl-thiazol-5-yl, 2,4-dimethyl-thiazol-5-yl, 2-thienyl, 4-morpholinyl, 4-methylpiperazin-1-yl, (C₁₋₄ alkyl)aminocarbonyl, trifluoromethyl, hydroxymethyl, (C₁₋₄ alkyl)sulfonylamino, (C₁₋₄ alkoxy)methylcarbonylamino, (C₁₋₄ alkyl)carbonylamino, phenylcarbonylamino, (C₁₋₄ alkoxy)carbonylamino, 2-furanylcarbonylamino, 2-thienylcarbonylamino, (C₁₋₄ alkyl)carbamyloxy, bis(methoxyethyl)amino, C₆H₅C(═N—CN)—NH—, 1H-pyrazol-5-ylamino, pyrimidin-2-ylamino, 1H-1,2,4-triazol-1-yl, thiazol-2-yl, thiazol-4-yl, thiazol-5-yl, and 5-phenyl-1,2,4-oxadiazol-3-yl, wherein any methyl, alkyl or alkoxy moiety in R¹⁰ is optionally substituted with one or more deuterium;

R⁴ is a group that is optionally substituted with one or more deuterium and is selected from 2,3-dihydrobenzofuran-5-yl, 3-oxo-2,3-dihydrobenzofuran-5-yl, chromanyl-6-yl, 4-oxo-chromanyl-6-yl, 4-oxo-4H-chromenyl-6-yl, 2,3-dihydrobenzo[b][1,4]dioxin-6-yl, 3,4-dihydro-2H-benzo[b][1,4]oxazin-6-yl, 3,4-dihydro-2H-benzo[b][1,4]oxazin-7-yl, 3-oxo-3,4-dihydro-2H-benzo[b][1,4]oxazin-7-yl, 3-oxo-3,4-dihydro-2H-benzo[b][1,4]oxazin-6-yl,

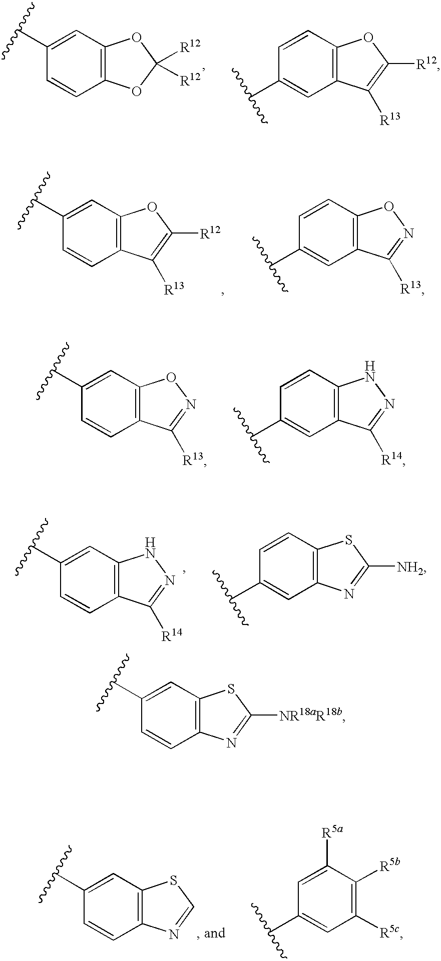

wherein:

each R⁵ is independently selected from hydrogen, —F, —NH₂, —NHC(O)C₁₋₆ alkyl optionally substituted with one or more deuterium, such as —NHC(O)CH₃ or —NHC(O)CD₃, —NHC(O)C₃₋₁₀cycloalkyl optionally substituted with one or more deuterium, —OH, —OCH₃, —OCD₃, —OCF₃, —OCHF₂, —CH₃, —CD₃, —CF₃, —CN, —CH₂—OCD₃, —CD₂-OCD₃, —CD₂-OCH₃, —CH₂OH, —CD₂OH, —OCH₂CH₃, —OCD₂CD₃, —OCH(CH₃)₂, —OCD(CH₃)₂, —OCD(CD₃)₂, and —OCH(CD₃)₂;

each R¹² and R¹⁴ (including the R¹⁶ and each R¹⁷ portion thereof) are as defined in Formula A;

R¹³ is selected from hydrogen, deuterium, —CH₃, —CD₃, —CH₂—O—CH₃, —CH₂—O-CD₃, —CD₂-O-CD₃, —CH₂-(1-piperidinyl), —CH₂-(4-morpholinyl), —(CH₂)_{w}-Q-C(O)-Q-R¹⁵, —(CD₂)_{w}-Q-C(O)-Q-R¹⁵, —CH₂—NH—X—R¹⁵, and —CD₂-NH—X—R¹⁵, wherein:

R¹⁵ and X are as defined in Formula A;

w is 1 or 2; and

each Q is independently NH, O, CH₂ or CD₂;

R^{18a} is selected from hydrogen, deuterium, —CH₃, —CD₃,

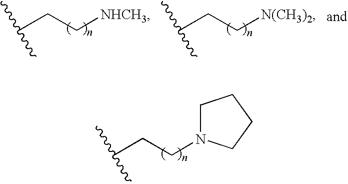

wherein n is 1 or 2, and wherein each of

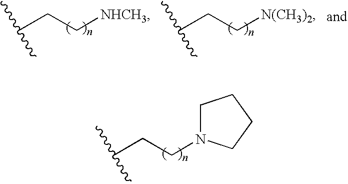

is optionally substituted on one or more carbon atoms with deuterium; and

R^{18b} is selected from hydrogen; deuterium; C₁₋₆ alkyl optionally substituted with one or more of halo, aryl, or heteroaryl; cyano; —COOH; —OC₁₋₆alkyl; —NH₂; —NH(C₁₋₆ alkyl); N(C₁₋₆alkyl)₂; a 3- to 10-membered cycloheteroalkyl; C₆₋₁₀ aryl, a 5- to 10membered heteroaryl; —C(O)OC₁₋₆ alkyl; —C(O)NHC₁₋₆alkyl;

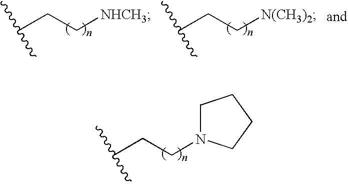

wherein n is 1 or 2, and wherein each of C₁₋₆ alkyl, —OC₁₋₆alkyl, —NHC₁₋₆alkyl, N(C₁₋₆alkyl)₂, —C(O)OC₁₋₆ alkyl, —C(O)NHC₁₋₆

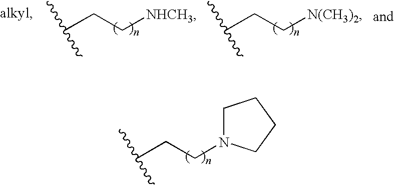

is optionally substituted on one or more carbon atoms with deuterium;
provided that when W is —O—, R^{2a} is hydrogen and R³ is isopropyl optionally substituted with one or more deuterium, then R⁴ is other than

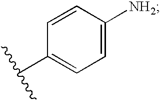

and further provided that at least one Y is deuterium or at least one of R¹, R^{2a}, R³ or R⁴ comprises a deuterium atom.

In a particular embodiment of Formula I, when W is —O—, and R³ is isopropyl optionally substituted with one or more deuterium, then R⁴ is other than

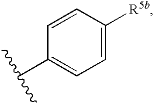

wherein R^{5b} is —OH, or NH₂.

In one embodiment of Formula A or Formula I, Y^{1a} and Y^{1b} are the same; Y^{5a} and Y^{5b} are the same; Y^{6a} and Y^{6b} are the same; Y^{7a} and Y^{7b} are the same; and R³ is selected from —CH(CH₃)₂, —CD(CH₃)₂, and —CD(CD₃)₂. In one aspect of this embodiment, W is —O—. In another aspect of this embodiment, W is —O—; each Y¹, Y², Y³, Y⁴, each Y⁵ and each Y⁶ is deuterium; and R³ is selected from —CH(CH₃)₂ and —CD(CD₃)₂. In another aspect of this embodiment, W is —O—; each Y¹, Y², Y³, Y⁴, each Y⁵ and each Y⁶ is deuterium; and R³ is —CD(CD₃)₂. In another aspect of this embodiment, W is —O—; each Y¹, and Y² are deuterium; Y³, Y⁴, each Y⁵ and each Y⁶ are the same; and R³ is —CD(CD₃)₂. In an alternate aspect, W is —O—; each Y¹ and Y² is deuterium; Y³, Y⁴, each Y⁵ and each Y⁶ is hydrogen; and R³ is selected from —CH(CH₃)₂ and —CD(CD₃)₂.

In one embodiment of Formula A or Formula I, R¹ is hydrogen. In one aspect of this embodiment, Y^{1a} and Y^{1b} are the same; Y^{5a} and Y^{5b} are the same; Y^{6a} and Y^{6b} are the same; Y^{7a} and Y^{7b} are the same; and R³ is selected from —CH(CH₃)₂, —CD(CH₃)₂, and —CD(CD₃)₂. In another aspect of this embodiment, W is —O—. In yet another aspect W is —O—; Y^{1a} and Y^{1b} are the same; Y^{5a} and Y^{5b} are the same; Y^{6a} and Y^{6b} are the same; Y^{7a} and Y^{7b} are the same; and R³ is selected from —CH(CH₃)₂, —CD(CH₃)₂, and —CD(CD₃)₂. In another aspect of this embodiment, W is —O—; each Y¹, Y², Y³, Y⁴, each Y⁵ and each Y⁶ is deuterium; and R³ is selected from —CH(CH₃)₂ and —CD(CD₃)₂. In another aspect of this embodiment, W is —O—; each Y¹, Y², Y³, Y⁴, each Y⁵ and each Y⁶ is deuterium; and R³ is —CD(CD₃)₂. In another aspect of this embodiment, W is —O—; each Y¹, and Y² are deuterium; Y³, Y⁴, each Y⁵ and each Y⁶ are the same; and R³ is —CD(CD₃)₂. In yet another aspect of this embodiment, W is —O—; each Y¹ and Y² is deuterium; Y³, Y⁴, each Y⁵ and each Y⁶ is hydrogen; and R³ is selected from —CH(CH₃)₂ and —CD(CD₃)₂.

In another embodiment of Formula A or Formula I, R¹ is —(C₁-C₁₁ alkylene)-R⁶, wherein:

the C₁-C₁₁ alkylene is optionally substituted by one or more groups independently selected from halo, cyano, —OH, ═O, —SH, —PO₃H, —PO₃(C₁₋₆ alkyl), ═N, —NH₂, NH(C₁-C₄ alkyl), N(C₁-C₄ alkyl)₂, —C₁-C₄ alkyl, cycloheteroalkyl, cycloheteroalkylalkyl, aryl, arylalkyl, heteroaryl, heteroarylalkyl, or a side group of a naturally occurring amino acid, and up to 4 methylene units in the C₁-C₁₁ alkylene are optionally and independently replaced with —O—, —S—, —S(O)—, S(O)₂—, —P(O)₂—, —P(O)(OH)—, —NH—, or —N(C₁-C₆ alkyl)-, provided that the terminal end of R¹ bonded to the oxygen is not oxygen or nitrogen; and

R⁶ is selected from hydrogen, —N(R⁷)(R⁷), optionally substituted C₁-C₈ alkyl, C₁-C₈ alkoxy, or a heteroaryl or cycloheteroalkyl, wherein the heteroaryl or cycloheteroalkyl can be optionally substituted with C₁-C₈ alkyl, wherein each R⁷ is independently selected from hydrogen, C₁-C₈ alkyl, and C₁-C₈ alkoxy.

Another embodiment provides a salt of a compound of Formula A or Formula I, wherein: R¹ is selected from R⁸—S(O)—OH and R⁸—P(O)(OH)₂; R⁸ is selected from a bond and —CH₂—O— optionally substituted with 1 to 2 substituents independently selected from C₁-C₃ alkyl; and the salt is selected from a lithium salt, a potassium salt, a barium salt, a sodium salt, a magnesium salt, an ammonium salt, a glycine salt, a lysine salt and an arginine salt. In one aspect of this embodiment R¹ is R⁸—P(O)(OH)₂ and the salt is selected from a sodium salt, a magnesium salt, and an ammonium salt.

In another embodiment of Formula A or Formula I, R¹ is —R⁸-(AA), wherein (AA) is an α-amino acid of (L)-configuration; and wherein R⁸ is selected from a bond and —CH₂—O— optionally substituted with 1 to 2 substituents independently selected from C₁-C₃ alkyl;. In one aspect of this embodiment, (AA) is selected from (L)-serine, (L)-lysine, (L)-tyrosine, (L)-valine, (L)-glutamic acid, (L)-aspartic acid, (L)-3-pyridylalanine, and (L)-histidine.

In yet another embodiment of Formula A or Formula I, R¹ is —R⁸—C(O)—R⁹, wherein R⁹ is hydrogen or C₁-C₃ alkyl; wherein the alkyl group in R⁹ is optionally substituted with cyano, hydroxyl, carboxy, alkoxy, amino, alkylamino, dialkylamino, cycloheteroalkyl, cycloheteroalkylalkyl, aryl, arylalkyl, heteroaryl, and heteroarylalkyl. In a more specific aspect of this embodiment, R⁹ is selected from methoxymethyl, methoxyethyl, 4-methylpiperazin-1-ylmethyl, piperazin-1-ylmethyl, morpholin-4-ylmethyl, carboxyethyl, aminoethyl, methylaminoethyl, and dimethylaminoethyl.

In yet another embodiment of Formula A or Formula I, R¹ is selected from —C(O)—CH₂—O—CH₂—C(O)—R¹⁹, —C(O)—CH₂—O—C(O)—CH₂—R¹⁹, or —C(O)—CH₂—O—CH₂CH₂—R¹⁹, wherein R¹⁹ is selected from —N(R⁷)(R⁷), —CH₂—R⁷, —(C₁-C₆ alkyl)-heteroaryl, —NH-heteroaryl, —(OCH₂CH₂)₁₋₃—H, heteroaryl, or —O—CH₂—C(O)—N(R⁷)(R⁷).

In still another embodiment of Formula A or Formula I, R¹ is selected from any one of:

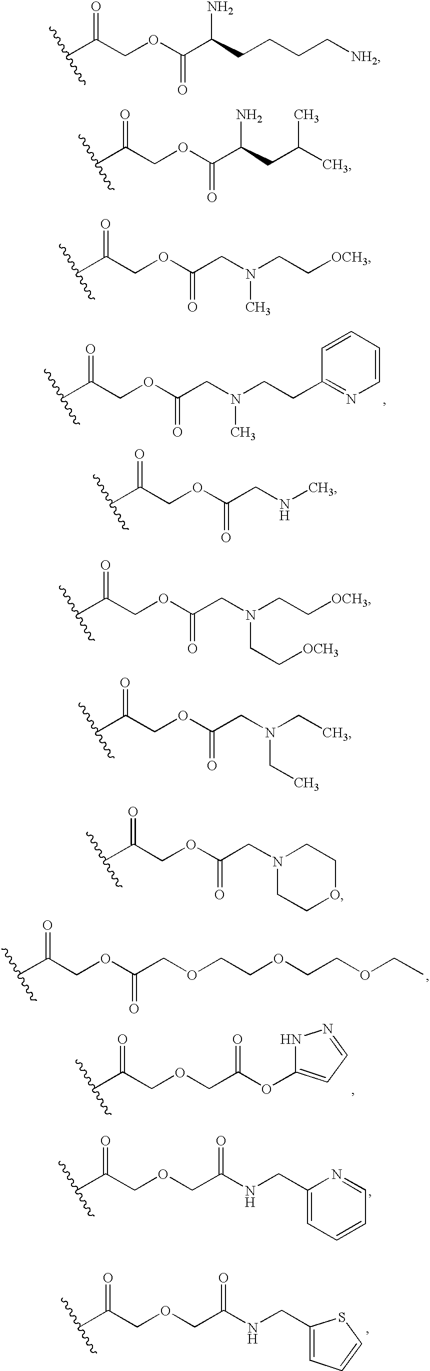

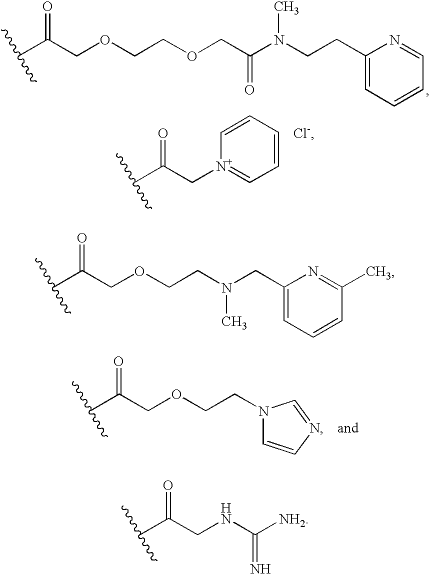

In another embodiment of Formula A or Formula I, R² or R^{2a} is H. In one aspect of this embodiment, W is —O—; each Y¹, Y², Y³, Y⁴, each Y⁵ and each Y⁶ is deuterium; R¹ is hydrogen; and R³ is —CD(CD₃)₂. In another aspect of this embodiment, W is —O—; each Y¹, and Y² are deuterium; Y³, Y⁴, each Y⁵ and each Y⁶ is hydrogen; R¹ is hydrogen; and R³ is —CD(CD₃)₂.

In an alternate embodiment of Formula A or Formula I, R² is —O—Z—R^{10a} or R^{2a} is —O—Z—R¹⁶. In one aspect of this embodiment, R¹⁰ or R^{10a} is selected from pyridyl, pyrazinyl, pyrazol-1-yl, 3,5-dimethylpyrazol-1-yl, isoxazol-3-yl, 5-methyl-isoxazol-3-yl, 2-methyl-thiazol-4-yl, 5-methyl-thiazol-4-yl, 2,5-dimethyl-thiazol-4-yl, 2-methyl-thiazol-5-yl, 4-methyl-thiazol-5-yl, 2,4-dimethyl-thiazol-5-yl, 4-morpholinyl, 4-piperazinyl, methylsulfonylamino, methoxymethylcarbonylamino, methylcarbamyloxy, and (C₁-C₄ alkoxy)carbonylamino, wherein any methyl or alkoxy moiety in R¹⁰ or R^{10a} is optionally substituted with one or more deuterium. In a more specific aspect of this embodiment, R¹⁰ or R^{10a} is selected from pyridyl, pyrazinyl, pyrazol-1-yl, 3,5-dimethylpyrazol-1-yl, 5-methyl-isoxazol-3-yl, 2-methyl-thiazol-4-yl, 5-methyl-thiazol-4-yl, 2,5-dimethyl-thiazol-4-yl, 2-methyl-thiazol-5-yl, and 4-methyl-thiazol-5-yl, 2,4-dimethyl-thiazol-5-yl, wherein any methyl in R¹⁰ or R^{10a} group is optionally substituted with one or more deuterium. In a still more specific aspect, W is —O—; each Y¹, Y², Y³, Y⁴, each Y⁵ and each Y⁶ is deuterium; R¹ is hydrogen; and R³ is —CD(CD₃)₂; Z is —CH₂— or —CD₂-; and R¹⁰ or R^{10a} is selected from

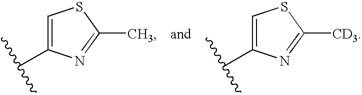

In another more specific aspect, W is —O—; each Y¹ and Y² is deuterium; Y³, Y⁴, each Y⁵ and each Y⁶ are hydrogen; R¹ is hydrogen; and R³ is —CD(CD₃)₂; Z is —CH₂— or —CD₂-; and R¹⁰ or R^{10a} is selected from

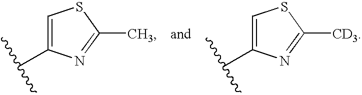

In another embodiment of Formula I, R⁴ is selected from:

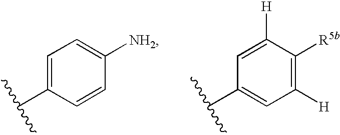

wherein R^{5b} is —NHC(O)C₁₋₆ alkyl optionally substituted with one or more deuterium or —NHC(O)C₃₋₁₀cycloalkyl optionally substituted with one or more deuterium,

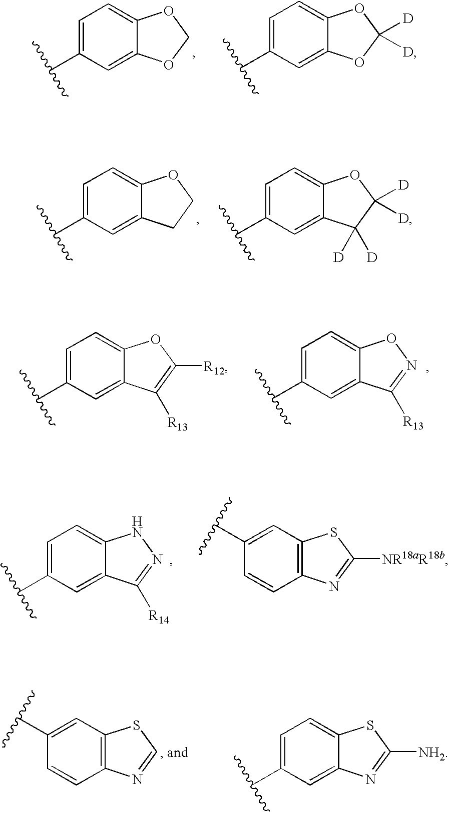

In a more specific aspect of this embodiment, W is —O—; each Y¹, Y², Y³, Y⁴, each Y⁵ and each Y⁶ is deuterium; R¹ is hydrogen; R³ is —CD(CD₃)₂; and R⁴ is selected from

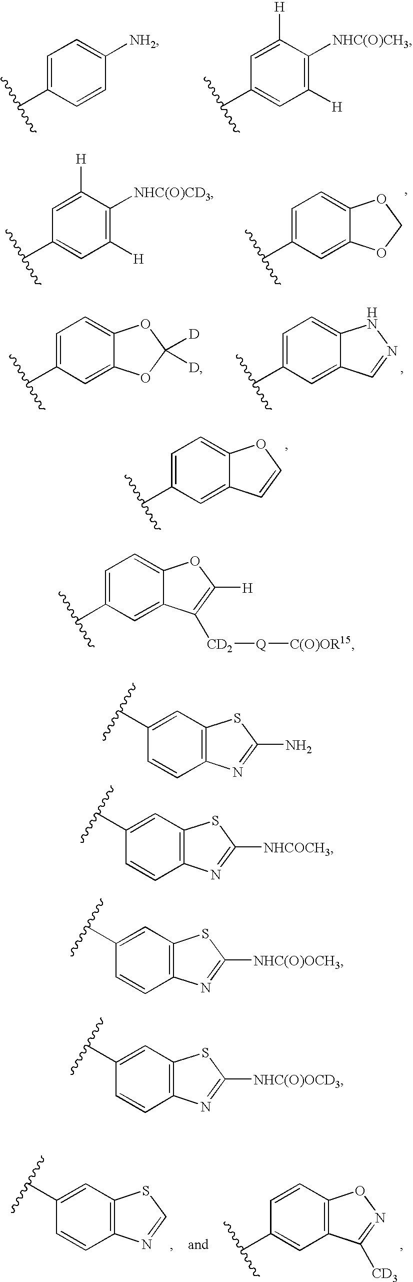

wherein Q is NH or O; and R¹⁵ is CD₃, CH₃, CD₂CD₃, CD₂CH₃, CH₂CD₃, or CH₂CH₃. In another more specific aspect of this embodiment, W is —O—; each Y¹, and Y² is deuterium; Y³, Y⁴, each Y⁵ and each Y⁶ is hydrogen; R¹ is hydrogen; R³ is —CD(CD₃)₂; and R⁴ is selected from

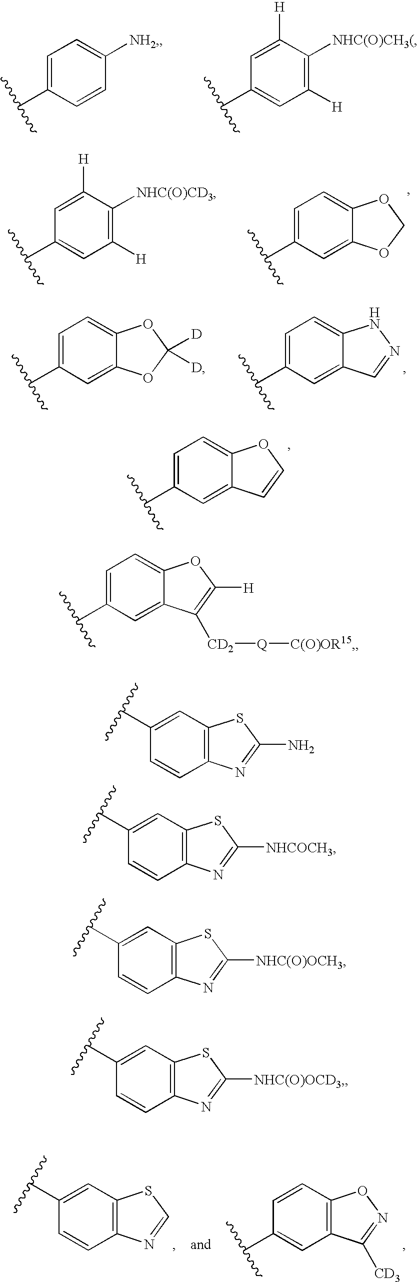

wherein Q is NH or O; and R¹⁵ is CD₃, CH₃, CD₂CD₃, CD₂CH₃, CH₂CD₃, or CH₂CH₃.

In one embodiment of Formula I, R⁴ is selected from any one of the following:

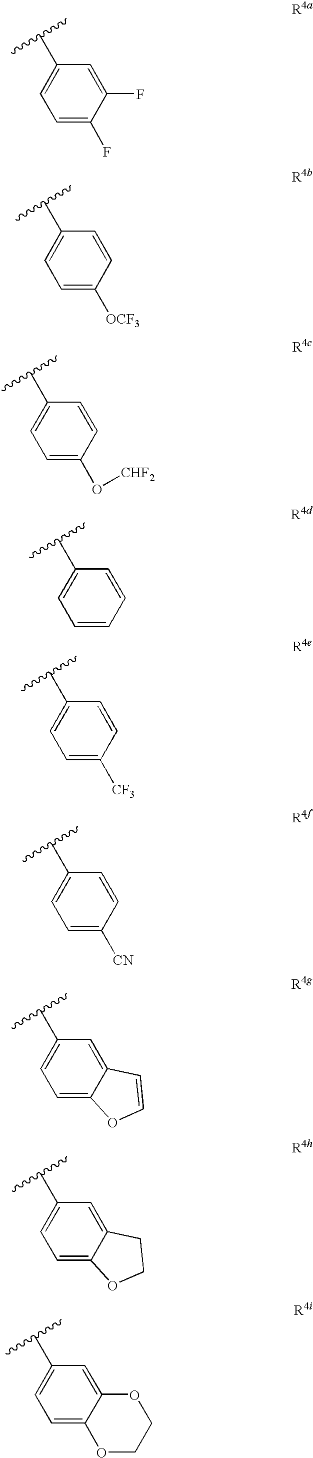

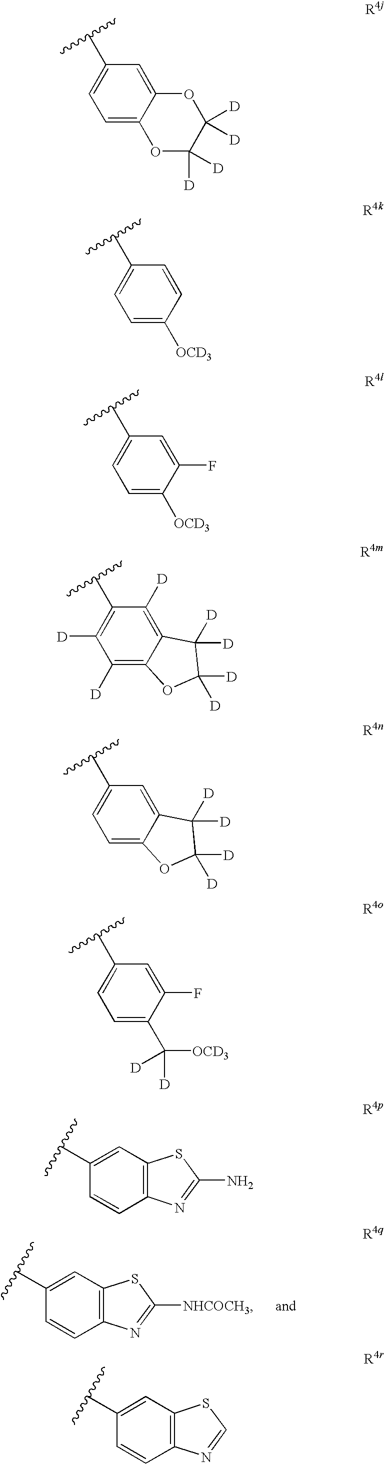

In one embodiment, R⁴ is R^{4c}. In another embodiment, R⁴ is R^{4g}. In another embodiment, R⁴ is R⁴ⁱ. In another embodiment, R⁴ is R^{4r}.

Examples of compounds of Formula I include the following compounds or pharmaceutically acceptable salts thereof:

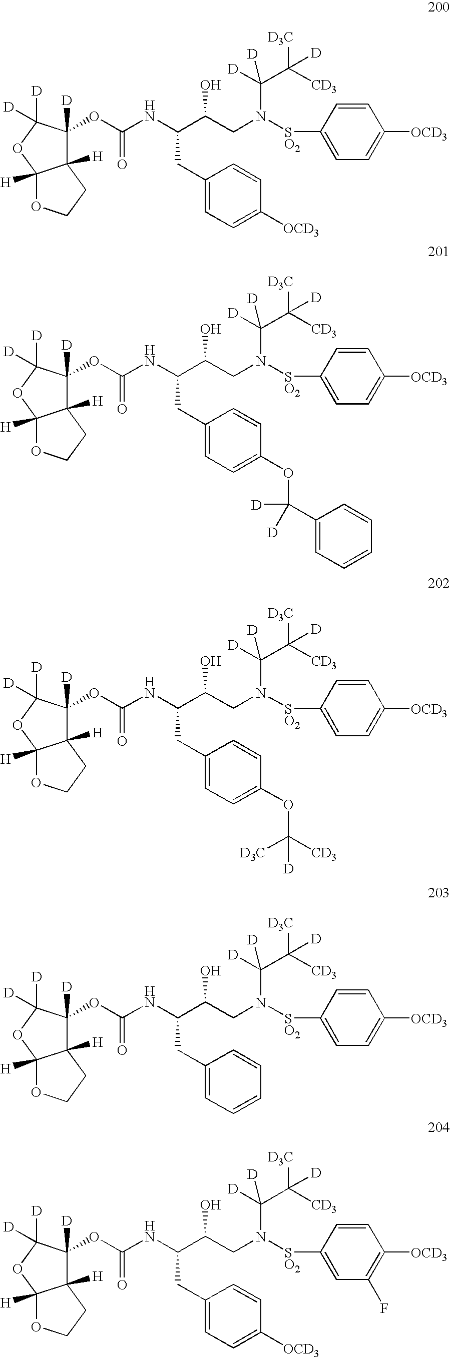

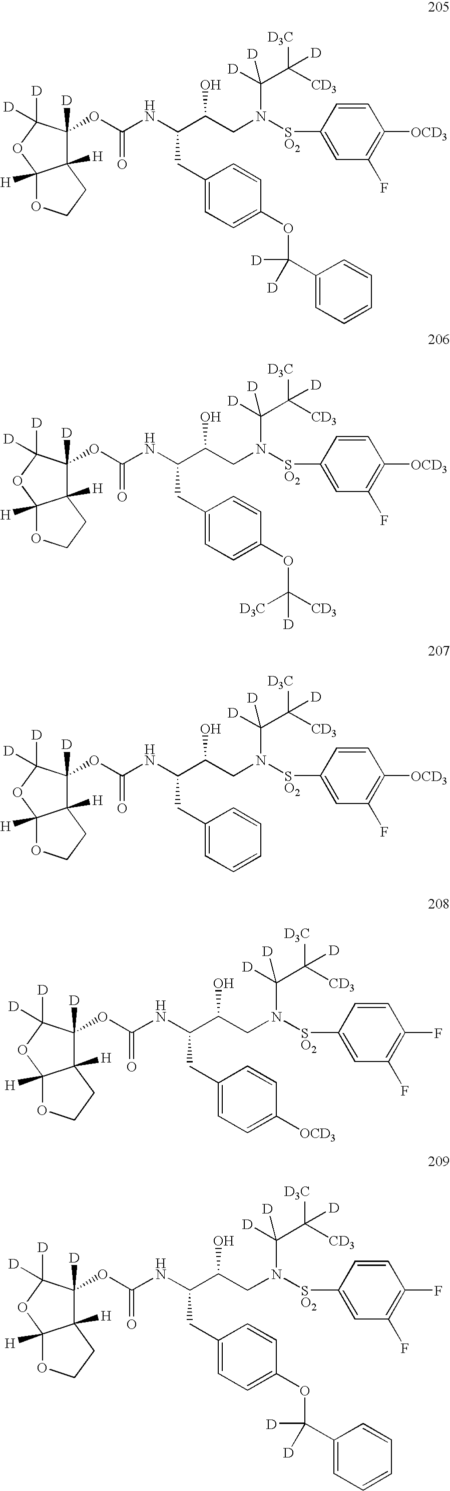

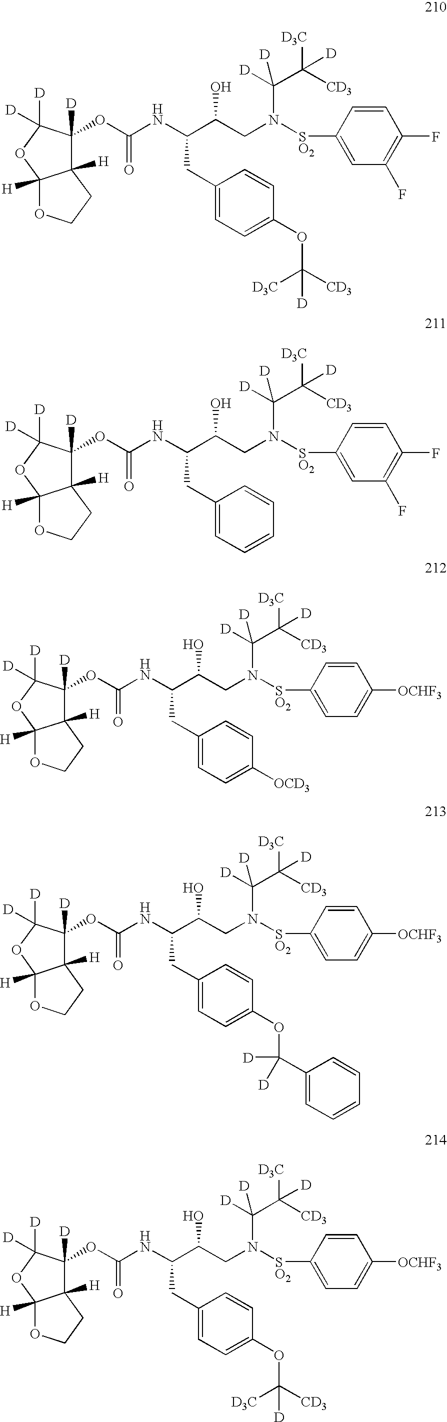

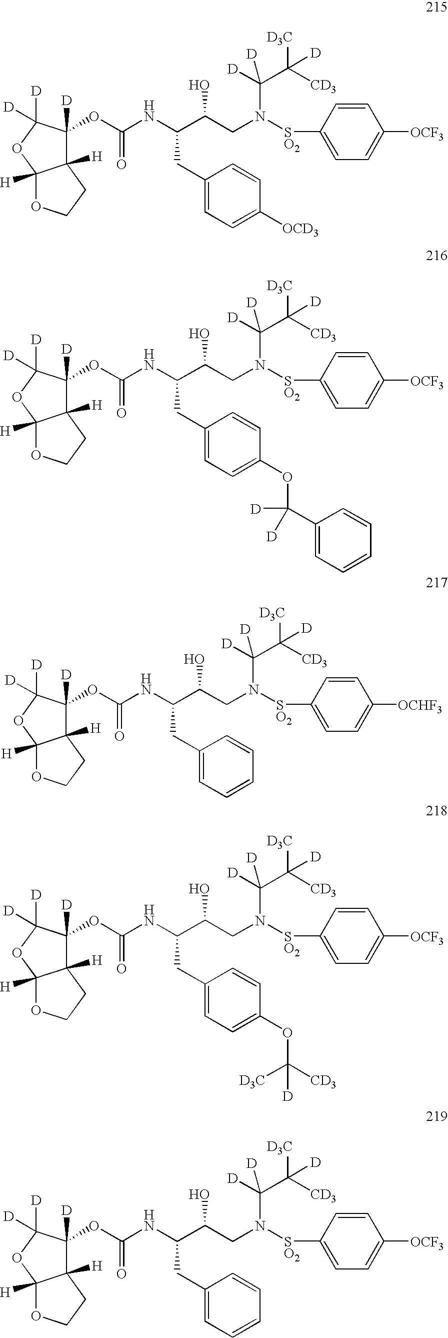

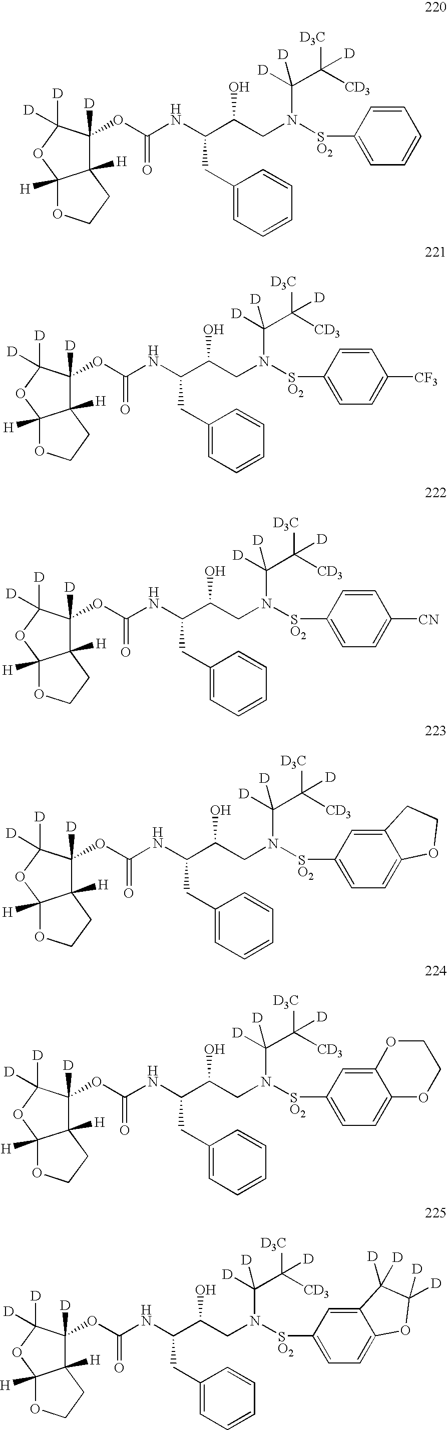

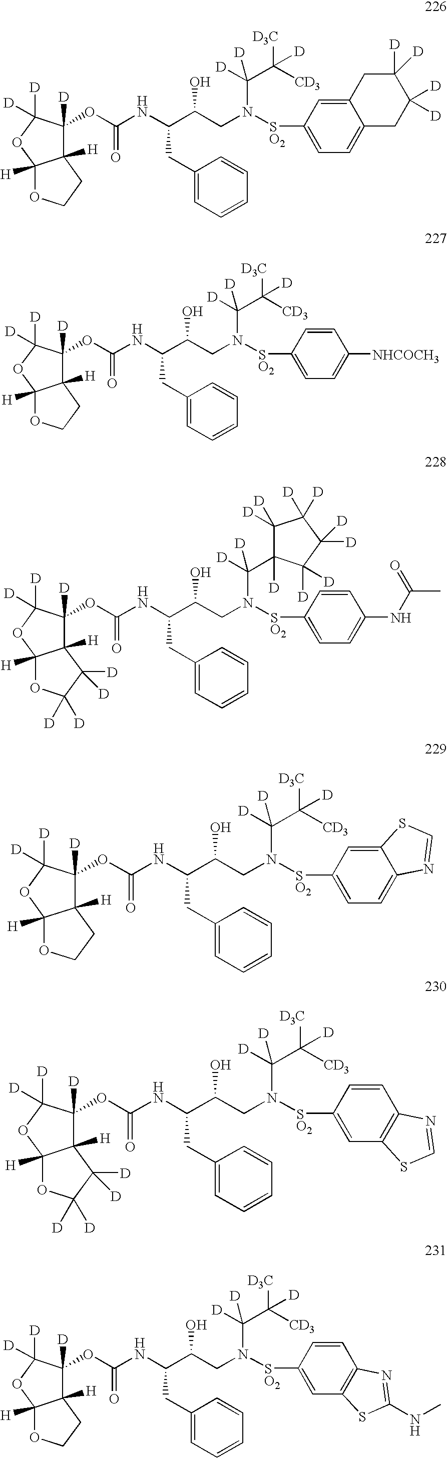

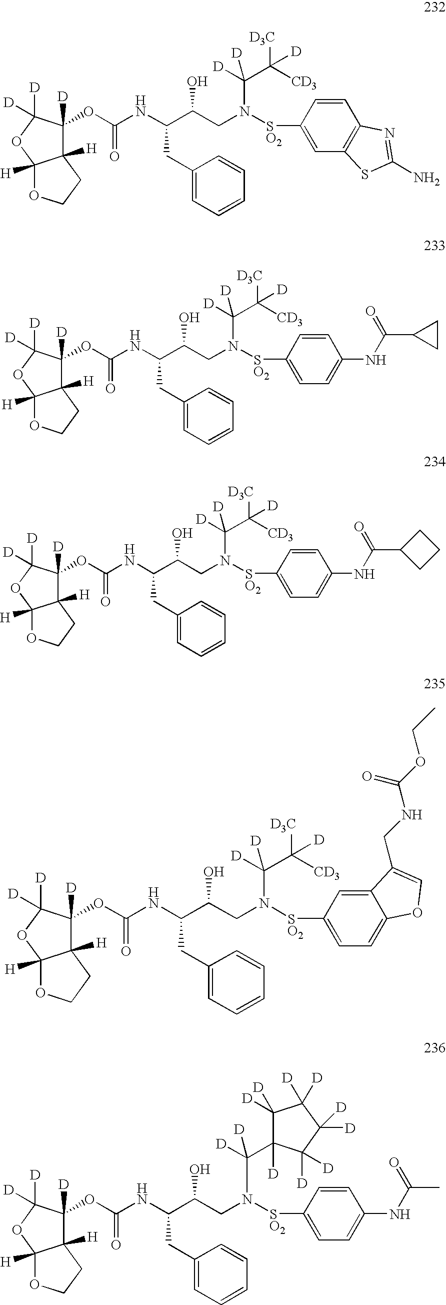

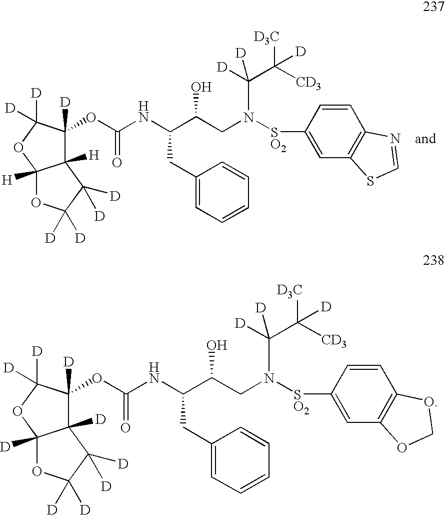

In one embodiment, the compound of Formula I is compound 103 or a pharmaceutically acceptable salt thereof

In one embodiment, the compound of Formula I is compound 106 or a pharmaceutically acceptable salt thereof.

In one embodiment, the compound of Formula I is compound 110 or a pharmaceutically acceptable salt thereof.

In one embodiment, the compound of Formula I is compound 115 or a pharmaceutically acceptable salt thereof.

In one embodiment, the compound of Formula I is compound 121 or a pharmaceutically acceptable salt thereof.

In one embodiment, the compound of Formula I is compound 128 or a pharmaceutically acceptable salt thereof.

In one embodiment, the compound of Formula I is compound 205 or a pharmaceutically acceptable salt thereof.

In one embodiment, the compound of Formula I is compound 208 or a pharmaceutically acceptable salt thereof

In one embodiment, the compound of Formula I is compound 212 or a pharmaceutically acceptable salt thereof.

In one embodiment, the compound of Formula I is compound 217 or a pharmaceutically acceptable salt thereof.

In one embodiment, the compound of Formula I is compound 223 or a pharmaceutically acceptable salt thereof.

In one embodiment, the compound of Formula I is compound 230 or a pharmaceutically acceptable salt thereof.

In one embodiment, the compound of Formula I is compound 238 or a pharmaceutically acceptable salt thereof.

In one embodiment, the compound of Formula I is a compound of the Formula Id:

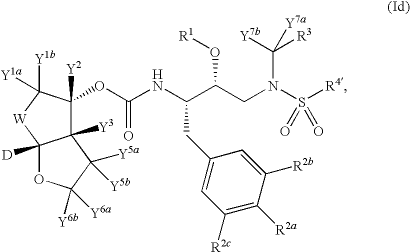

or a pharmaceutically acceptable salt thereof

In one embodiment, the compound of Formula I is a compound of the Formula Ie:

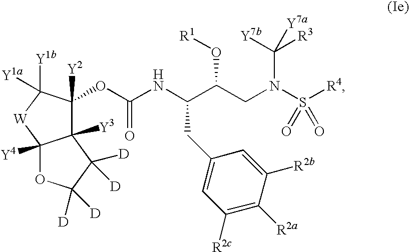

or a pharmaceutically acceptable salt thereof

In one embodiment, the compound of Formula I is a compound of the Formula If:

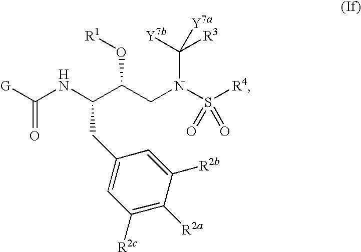

or a pharmaceutically acceptable salt thereof, wherein G is a group selected from:

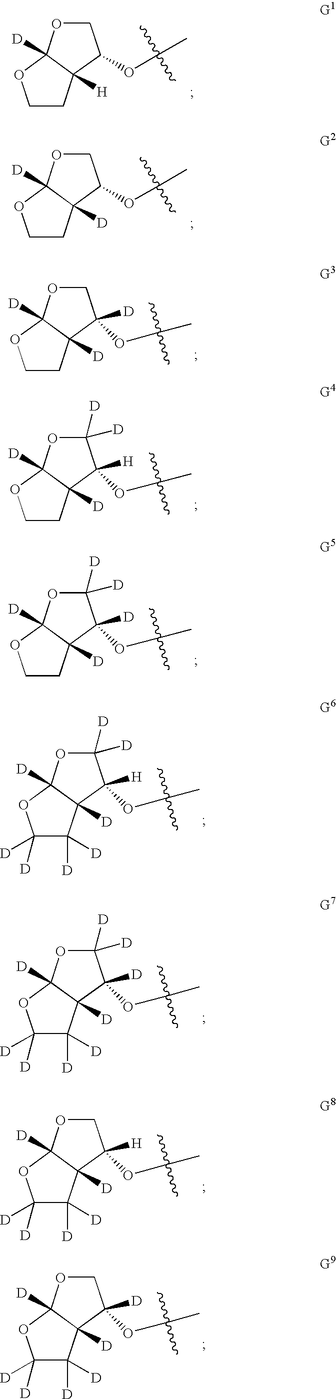

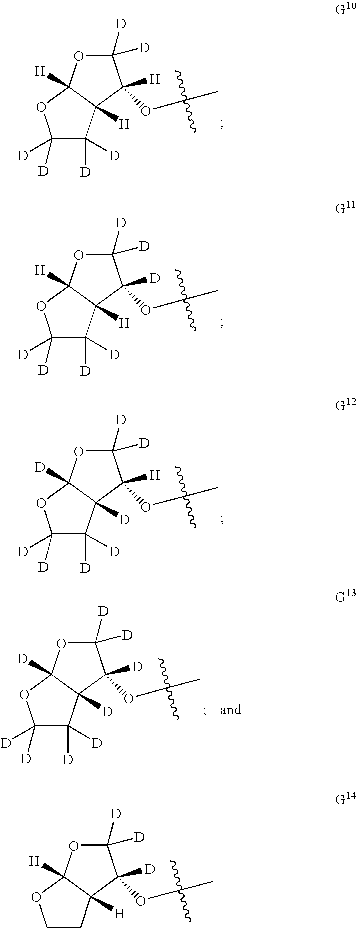

In one embodiment, the group G is selected from G¹¹, G¹³ and G¹⁴.

In one embodiment, the present invention provides compounds of Formula I represented by structural Formula Ig:

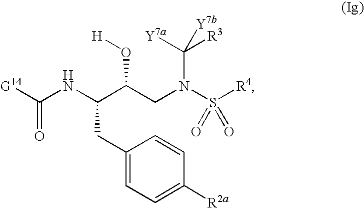

or a pharmaceutically acceptable salt thereof, wherein:

R^{2a} is selected from: hydrogen, —OCD₃, —OCD₂C₆H₅, —O(CD₃)₃,

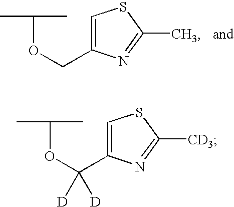

R³ is selected from: —CH₂CH(CH₃)₂, —CD₂CD(CH₃)₂, —CD₂CD(CD₃)₂, —CH(CH₃)₂, —CD(CH₃)₂, and —CD(CD₃)₂; and

R⁴ is selected from:

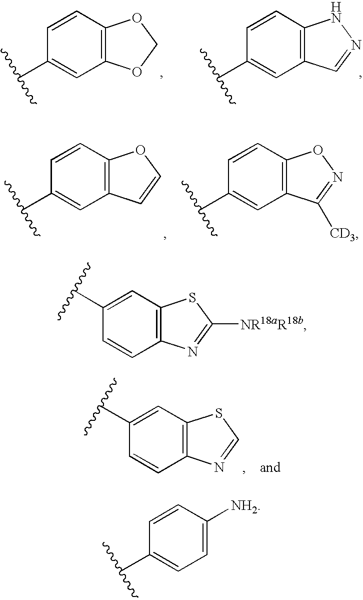

In one aspect of Formula Ia, Id, Ie, If or Ig, Y^{7a} and Y^{7b} are the same. In one aspect both Y^{7a} and Y^{7b} are deuterium.

In another embodiment, the present invention provides a compound of Formula II:

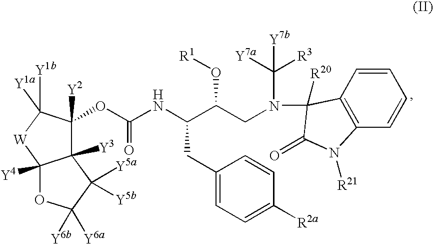

### or Formula A-II:

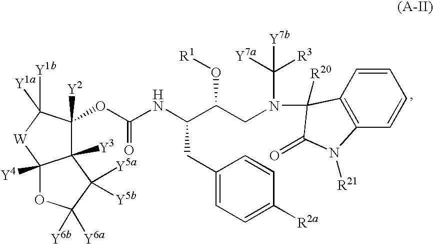

or a pharmaceutically acceptable salt of either of the foregoing, wherein:

W, each Y, R¹, R^{2a}, and R³ are as defined for Formula I;

R² is as defined for Formula A; and

R²⁰ is selected from methyl and C₂-C₄ alkenyl, wherein R²⁰ is optionally substituted with one or more deuterium;

R²¹ is selected from hydrogen, —CH₃, and —CD₃; and provided that at least one Y is deuterium or at least one of R¹, R³, R²⁰, R²¹ or in the case of Formula II, R^{2a} and in the case of Formula AII, R² comprises a deuterium atom.

In one embodiment of Formula II or Formula A-II, Y^{1a} and Y^{1b} are the same; Y^{5a} and Y^{5b} are the same; Y^{6a} and Y^{6b} are the same; Y^{7a} and Y^{7b} are the same; and R³ is selected from —CH₂CH(CH₃)₂, —CD₂CD(CH₃)₂, —CD₂CD(CD₃)₂, —CH(CH₃)₂, —CD(CH₃)₂, and —CD(CD₃)₂. In one aspect of this embodiment, W is —O—. In another aspect, W is —O—. each Y¹, Y², Y³, Y⁴, each Y⁵ and each Y⁶ is deuterium; and R³ is selected from —CH(CH₃)₂ and —CD(CD₃)₂. In another aspect of this embodiment, W is —O—; each Y¹, Y², Y³, Y⁴, each Y⁵ and each Y⁶ is deuterium; and R³ is —CD(CD₃)₂. In another aspect of this embodiment, W is —O—; each Y¹, and Y² are deuterium; Y³, Y⁴, each Y⁵ and each Y⁶ are the same; and R³ is —CD(CD₃)₂. In an alternate aspect, W is —O—; each Y¹ and Y² is deuterium; Y³, Y⁴, each Y⁵ and each Y⁶ is hydrogen; and R³ is selected from —CH₂CH(CH₃)₂—CD₂CD(CD₃)₂, —CH(CH₃)₂ and —CD(CD₃)₂.

In one embodiment of Formula II or Formula A-II, R¹ is hydrogen. In one aspect of this embodiment, Y^{1a} and Y^{1b} are the same; Y^{5a} and Y^{5b} are the same; Y^{6a} and Y^{6b} are the same; Y^{7a} and Y^{7b} are the same; and R³ is selected from —CH(CH₃)₂, —CD(CH₃)₂, and —CD(CD₃)₂. In another aspect of this embodiment, W is —O—. In yet another aspect W is —O—; Y^{1a} and Y^{1b} are the same; Y^{5a} and Y^{5b} are the same; Y^{6a} and Y^{6b} are the same; Y^{7a} and Y^{7b} are the same; and R³ is selected from —CH(CH₃)₂, —CD(CH₃)₂, and —CD(CD₃)₂. In another aspect of this embodiment, W is —O—; each Y¹, Y², Y³, Y⁴, each Y⁵ and each Y⁶ is deuterium; and R³ is selected from —CH(CH₃)₂ and —CD(CD₃)₂. In another aspect of this embodiment, W is —O—; each Y¹, Y², Y³, Y⁴, each Y⁵ and each Y⁶ is deuterium; and R³ is —CD(CD₃)₂. In yet another aspect of this embodiment, W is —O—; each Y¹ and Y² is deuterium; Y³, Y⁴, each Y⁵ and each Y⁶ is hydrogen; and R³ is selected from —CH(CH₃)₂ and —CD(CD₃)₂. In a further aspect of this embodiment, W is —O—; each Y¹ and Y² is deuterium; Y³, Y⁴, each Y⁵ and each Y⁶ are the same; and R³ is selected from —CH₂CH(CH₃)₂—CD₂CD(CD₃)₂, —CH(CH₃)₂ and —CD(CD₃)₂.

Another embodiment provides a compound of Formula II or Formula A-II where R¹ is —(C₁-C₁₁ alkylene)-R⁶, wherein:

the C₁-C₁₁ alkylene is optionally substituted by one or more groups independently selected from halo, cyano, —OH, ═O, —SH, —PO₃H, —PO₃(C₁₋₆ alkyl), ═N, —NH₂, NH(C₁-C₄ alkyl), N(C₁-C₄ alkyl)₂, —C₁-C₄ alkyl, cycloheteroalkyl, cycloheteroalkylalkyl, aryl, arylalkyl, heteroaryl, heteroarylalkyl, or a side group of a naturally occurring amino acid, and up to 4 methylene units in the C₁-C₁₁ alkylene are optionally and independently replaced with —O—, —S—, —S(O)—, S(O)₂—, —P(O)₂—, —P(O)(OH), —NH—, and —N(C₁-C₆ alkyl)-, provided that the terminal end of R¹ bonded to the oxygen is not oxygen or nitrogen; and

R⁶ is selected from hydrogen, —N(R⁷)(R⁷), optionally substituted C₁-C₈ alkyl, C₁-C₈ alkoxy, heteroaryl or cycloheteroalkyl, wherein the heteroaryl or cycloheteroalkyl are optionally substituted with C₁-C₈ alkyl, wherein each R⁷ is independently selected from hydrogen, C₁-C₈ alkyl, and C₁-C₈ alkoxy.

Another embodiment provides a salt of a compound of Formula II or Formula A-II, wherein: R¹ is selected from R⁸—S(O)—OH and R⁸—P(O)(OH)₂; R⁸ is selected from a bond and —CH₂—O— optionally substituted with 1 to 2 substituents independently selected from C₁-C₃ alkyl; and the salt is selected from a lithium salt, a potassium salt, a barium salt, a sodium salt, a magnesium salt, an ammonium salt, a glycine salt, a lysine salt and an arginine salt. In one aspect of this embodiment R¹ is R⁸—P(O)(OH)₂ and the salt is selected from a sodium salt, a magnesium salt, and an ammonium salt.

In another embodiment of Formula II or Formula A-II, R¹ is —R⁸-(AA), wherein (AA) is an α-amino acid of (L)-configuration and R⁸ is selected from a bond and —CH₂—O-optionally substituted with 1 to 2 substituents independently selected from C₁-C₃ alkyl. In one aspect of this embodiment, (AA) is selected from (L)-serine, (L)-lysine, (L)-tyrosine, (L)-valine, (L)-glutamic acid, (L)-aspartic acid, (L)-3-pyridylalanine, and (L)-histidine.

In yet another embodiment of Formula II or Formula A-II, R¹ is —R⁸—C(O)—R⁹, wherein R⁸ is selected from a bond and —CH₂—O— optionally substituted with 1 to 2 substituents independently selected from C₁-C₃ alkyl and R⁹ is hydrogen or C₁-C₃ alkyl; wherein the alkyl group in R⁹ is optionally substituted with cyano, hydroxyl, carboxy, alkoxy, amino, alkylamino, dialkylamino, cycloheteroalkyl, cycloheteroalkylalkyl, aryl, arylalkyl, heteroaryl, and heteroarylalkyl. In a more specific aspect of this embodiment, R⁹ is selected from methoxymethyl, methoxyethyl, 4-methylpiperazin-1-ylmethyl, piperazin-1-ylmethyl, morpholin-4-ylmethyl, carboxyethyl, aminoethyl, methylaminoethyl, and dimethylaminoethyl.

In yet another embodiment of Formula II or Formula A-II, R¹ is selected from —C(O)—CH₂—O—CH₂—C(O)—R¹⁹, —C(O)—CH₂—O—C(O)—CH₂—R¹⁹, or —C(O)—CH₂—O—CH₂CH₂—R¹⁹, wherein R¹⁹ is selected from —N(R⁷)(R⁷), —CH₂—R⁷, —(C₁-C₆ alkyl)-heteroaryl, —NH-heteroaryl, —(OCH₂CH₂)₁₋₃—H, heteroaryl, or —O—CH₂—C(O)—N(R⁷)(R⁷). In still another embodiment, R¹ is selected from any one of:

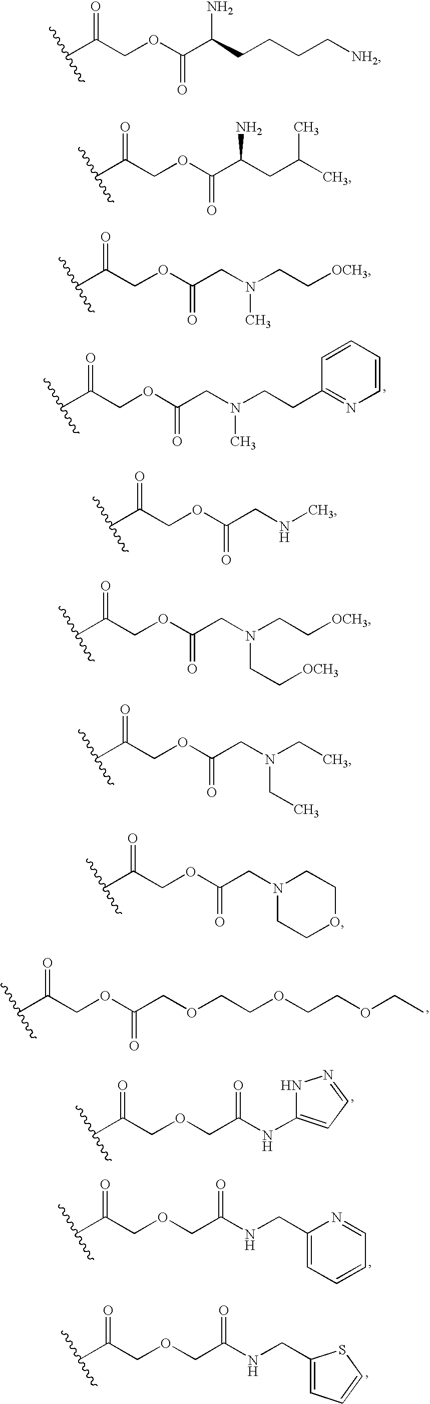

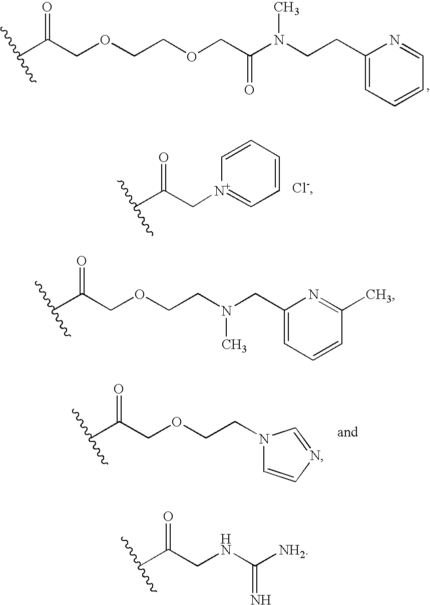

In another embodiment of Formula II or Formula A-II, R^{2a} or R² is H. In one aspect of this embodiment, W is —O—; each Y¹ and Y² are deuterium; Y³, Y⁴, each Y⁵ and each Y⁶ are the same; R¹ is hydrogen; and R³ is —CD(CD₃)₂.

In an alternate embodiment of Formula II or Formula A-II, R^{2a} is —O—Z—R¹⁰, or R² is —O—Z—R^{10a}, wherein R¹⁰ is as defined for Formula I and R^{10a} is as defined for Formula A. In one aspect of this embodiment, R¹⁰ or R^{10a} is selected from pyridyl, pyrazinyl, pyrazol-1-yl, 3,5-dimethylpyrazol-1-yl, isoxazol-3-yl, 5-methyl-isoxazol-3-yl, 2-methyl-thiazol-4-yl, 5-methyl-thiazol-4-yl, 2,5-dimethyl-thiazol-4-yl, 2-methyl-thiazol-5-yl, 4-methyl-thiazol-5-yl, 2,4-dimethyl-thiazol-5-yl, 4-morpholinyl, 4-piperazinyl, methylsulfonylamino, methoxymethylcarbonylamino, methylcarbamyloxy, and (C₁-C₄ alkoxy)carbonylamino, wherein any methyl or alkoxy moiety in R¹⁰ or R^{10a} is optionally substituted with one or more deuterium. In a more specific aspect of this embodiment, R¹⁰ or R^{10a} is selected from pyridyl, pyrazinyl, pyrazol-1-yl, 3,5-dimethylpyrazol-1-yl, 5-methyl-isoxazol-3-yl, 2-methyl-thiazol-4-yl, 5-methyl-thiazol-4-yl, 2,5-dimethyl-thiazol-4-yl, 2-methyl-thiazol-5-yl, and 4-methyl-thiazol-5-yl, 2,4-dimethyl-thiazol-5-yl, wherein any methyl in R¹⁰ or R^{10a} group is optionally substituted with one or more deuterium. In a still more specific aspect, W is —O—; each Y¹, and Y² are deuterium; Y³, Y⁴, each Y⁵ and each Y⁶ are the same; R¹ is hydrogen; and R³ is —CD(CD₃)₂; Z is —CH₂— or —CD₂—; and R¹⁰ or R^{10a} is selected from

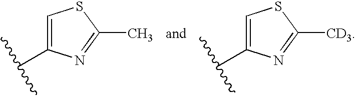

In still another embodiment of Formula II or Formula A-II, R²⁰ is selected from methyl and allyl.

The present invention provides additionally for novel intermediates of Formula IIIa:

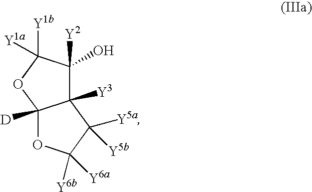

wherein each Y is independently selected from hydrogen and deuterium.

Examples of intermediates of Formula IIIa include:

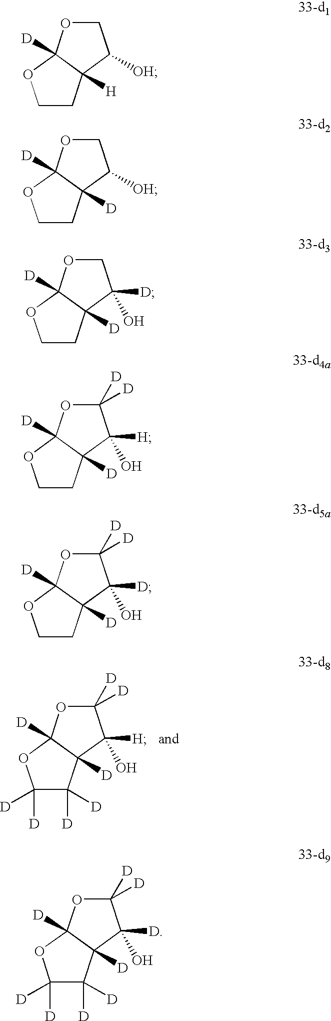

The present invention also provides for novel intermediates of Formula IIIb:

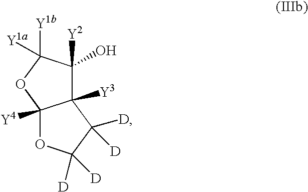

wherein each Y is independently selected from hydrogen and deuterium.

Examples intermediates of Formula IIIb include:

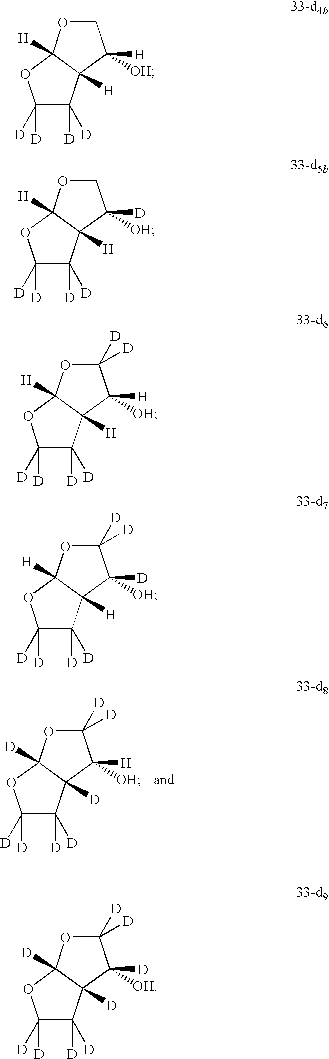

In another set of embodiments, any atom not designated as deuterium in any of the embodiments set forth above is present at its natural isotopic abundance.

The synthesis of compounds of Formula A or Formula I can be readily achieved by synthetic chemists of ordinary skill following known methods for making darunavir and utilizing corresponding deuterated and optionally, other isotope-containing reagents and/or intermediates to synthesize the compounds delineated herein, or invoking standard synthetic protocols known in the art for introducing isotopic atoms to a chemical structure. Relevant procedures and intermediates are disclosed, for instance in Ghosh, A K et al., J Org Chem, 2004, 69: 7822-7829; Ghosh, A K et al., J Med Chem, 2005, 48: 1813-1822; Ghosh, A K et al., J Med Chem, 2006, 49: 5252-5261; Doan, B D et al., US Patent App Pub No US 2005/0261507; Eissenstat, M et al., PCT publication No. WO2005110428; Hale, M et al., PCT publication WO2000076961; Desai, M et al., PCT publication WO2008013834; and Martin et al., US Patent App Pub No US 2006/0148865.

### Exemplary Synthesis

A convenient method for synthesizing compounds of any of Formulae A or Formula I is depicted in Schemes 1-10, below. In each of the schemes references to R^{2a} are interchangeable with R², references to R⁴ are interchangeable with R^{4′}, and references to R¹⁰ are interchangeable with R^{10a}.

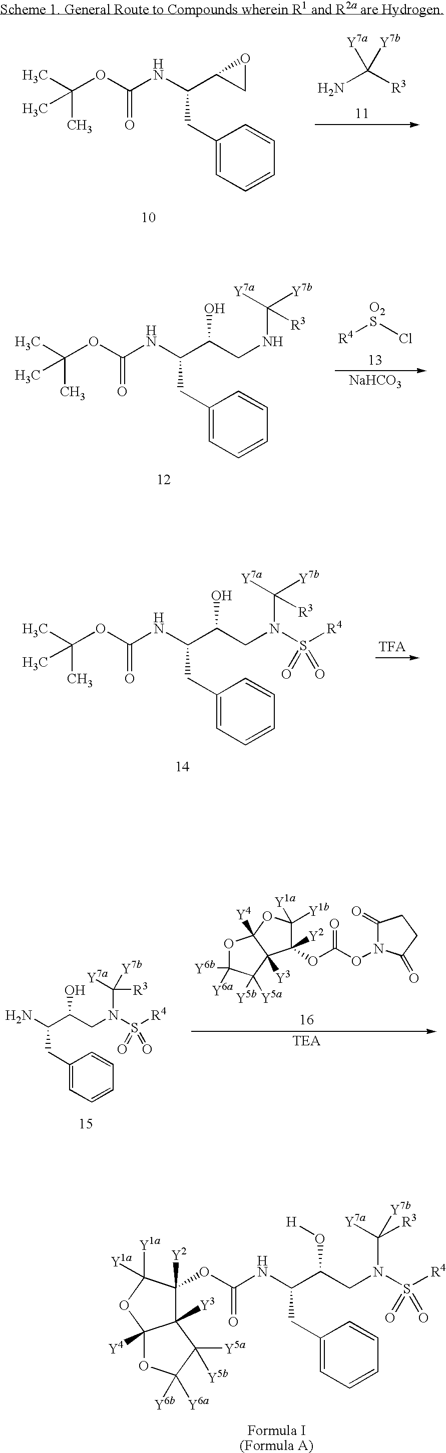

Scheme 1 above shows a general route to prepare compounds of Formula A or Formula I. Commercially available chiral epoxide (10) is opened with the appropriately deuterated amine 11 in hot isopropanol to provide the secondary amine 12. This amine 12 is then reacted with the appropriately deuterated sulfonyl chloride 13 and NaHCO₃ in dichloromethane to provide the sulfonamide 14. Trifluoroacetic acid treatment removes the Boc group to provide amine 15, which is then reacted with the appropriately deuterated mixed carbonate 16 and triethylamine (“TEA”) in dichloromethane to provide compounds of Formula I, wherein R¹ and R^{2a} are hydrogen or Formula A , wherein R¹ and R² are hydrogen.

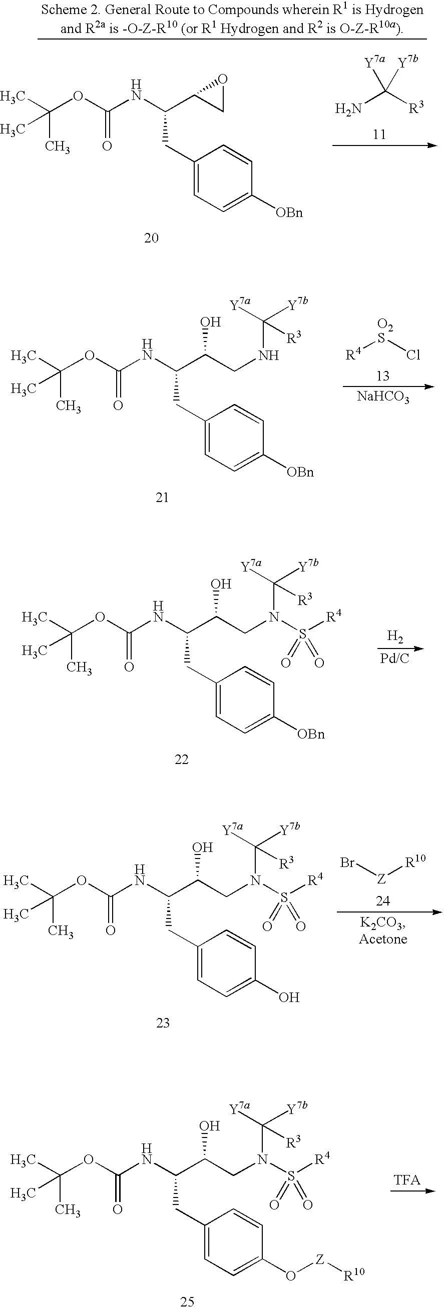

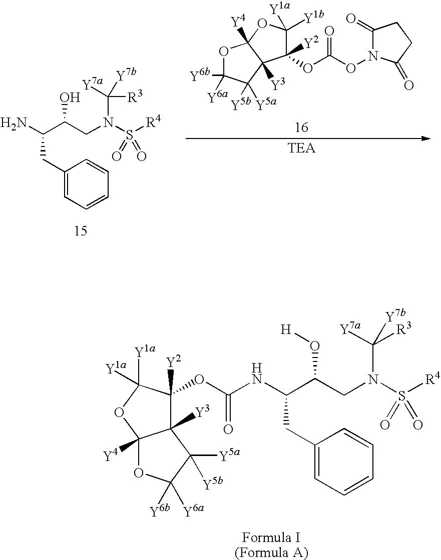

Compounds of Formula I where R^{2a} is O—Z—R¹⁰ (or of Formula A where R² is O—Z—R^{10a}) can be prepared as depicted in Scheme 2. The commercially available chiral epoxide (20) is opened with the appropriately deuterated amine 11 in hot isopropanol to provide the secondary amine 21. This amine 21 is then reacted with the appropriately deuterated sulfonyl chloride 13 and NaHCO₃ in dichloromethane to provide the sulfonamide 22, which is then debenzylated by hydrogenation over palladium on carbon to yield phenol 23. This phenol 23 is then alkylated with the desired bromide 24 to provide 25. Trifluoroacetic acid treatment removes the Boc group to provide 26, which is then reacted with the appropriate deuterated mixed carbonate 16 and TEA in dichloromethane to provide the desired compounds.

Other chiral epoxides may be used instead of chiral epoxides 10 and 20 in Schemes 1 and 2, respectively. Examples of syntheses of chiral epoxides are shown in Scheme 11 herein below.

Schemes 3A and 3B. Synthesis of an Appropriately Deuterated Mixed Carbonate 16.

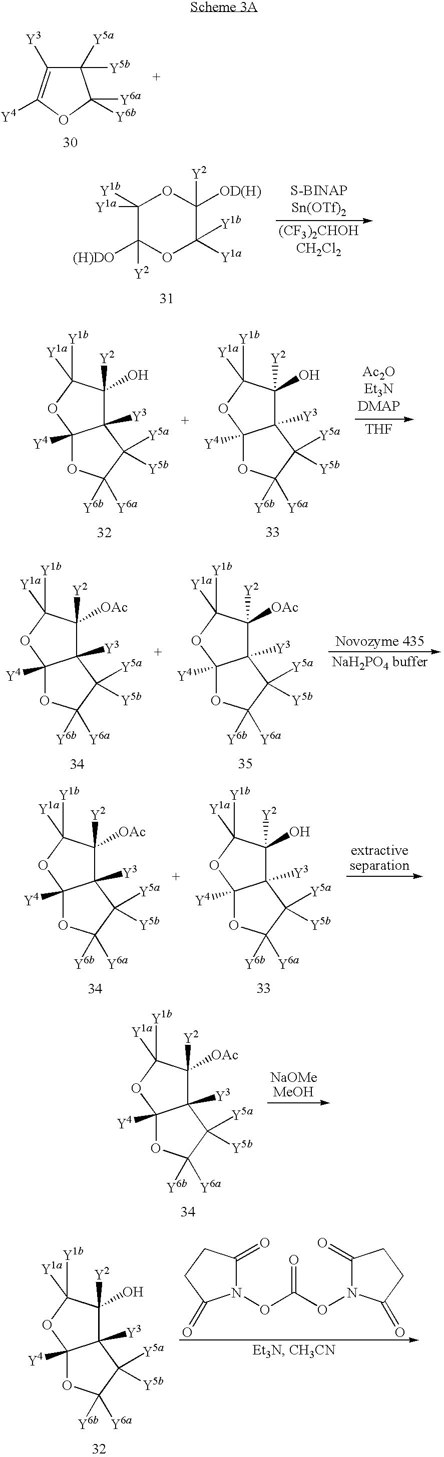

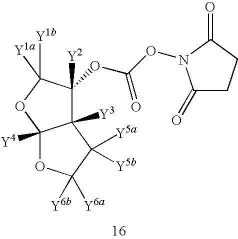

The appropriately deuterated mixed carbonate intermediate 16 can be prepared as shown in Scheme 3A and according to the procedures disclosed by Yu, R H et al, Org Proc Res Dev 2007, 11: 972 using the appropriately deuterated materials. The appropriately deuterated dihydrofuran 30 (the various deuterated forms of 30 can be prepared from succinic anhydride, from dihydrofuran, and from γ-butyrolactone as described in Keay, B A et al, J Org Chem 2007, 72: 7252-7259) is reacted with the deuterated glycolaldehyde dimer 31 (the glycolaldehyde 31, where each Y is deuterium, can be prepared from dihydroxyfumaric acid or dihydroxymaleic acid by thermal decarboxylation in D₂O as described in Wong, C—H and Whitesides, G M, J Am Chem Soc 1983, 105: 5012-5014) in the presence of S-BINAP and tin triflate to yield an enantiomeric mixture of bisfuranols 32 and 33. Acylation of hydroxy cis-bisfurans 32 and 33 using acetic anhydride affords the enantiomeric mixture of cis-bisfuran acetates 34 and 35. Hydrolysis of the mixture of 34 and 35 using the lipase Novozyme 435 from Candida antarctica (CAS No. 9001-62-1) selectively hydrolyzes acetate enantiomer 35 back to alcohol 33, while leaving acetate enantiomer 34 unchanged. Acetate 34 is isolated by subjecting the mixture of alcohol 33 and acetate 34 to organic extraction. Hydrolysis of 34 in base affords the enantiopure cis-bisfuran alcohol 32, which can then be converted to the mixed carbonate 16 by reaction with disuccinimidyl carbonate and triethylamine in acetonitrile as described by Ghosh, A K et al, in J Org Chem 2004, 69: 7822-7829.

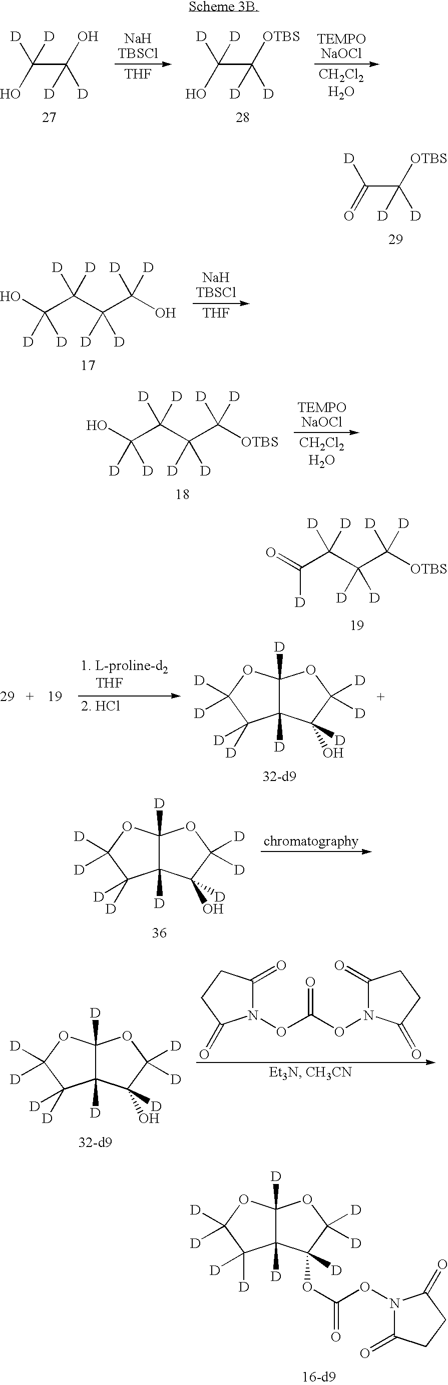

A perdeuterated mixed carbonate (16-d9) is prepared as shown in Scheme 3B. Commercially available ethylene glycol-d4 (27) is converted to the perdeuterated tert-butyldimethylsilyloxyethanol 28 by treatment with tert-butyldimethylsilyl chloride (“TBSCl”) in THF and sodium hydride. The ethanol 28 is converted to the acetaldehyde 29 by treatment with sodium bromide, sodium carbonate and 2,2,6,6-tetramethylpiperidine 1-oxyl (“TEMPO”), followed by addition of NaOCl.

Separately, commercially available 1,4-Butane-d8-diol (17) is protected with by treatment with tert-butyldimethylsilyl chloride (“TBSCl”) in THF and sodium hydride. The resulting 4-(tert-Butyldimethylsilyloxy)butan-d4-diol 18 is converted to the aldehyde 19 by treatment with sodium bromide, sodium carbonate and 2,2,6,6-tetramethylpiperidine 1-oxyl (“TEMPO”), followed by addition of NaOCl.

The two TBS-protected aldehydes 29 and 19 are combined with L-proline-d2 (prepared by stirring L-proline in MeOD) in THF. This is followed by acid treatment with HCl which produces an diastereomeric mixture of perdeuterated bisfuranols 32-d₉ and 36. The desired isomer 32-d₉ is isolated by chromatography and converted to the perdeuterated mixed carbonate 16-d₉ by reaction with disuccinimidyl carbonate and triethylamine in acetonitrile as described by Ghosh, A K et al, in J Org Chem 2004, 69: 7822-7829.

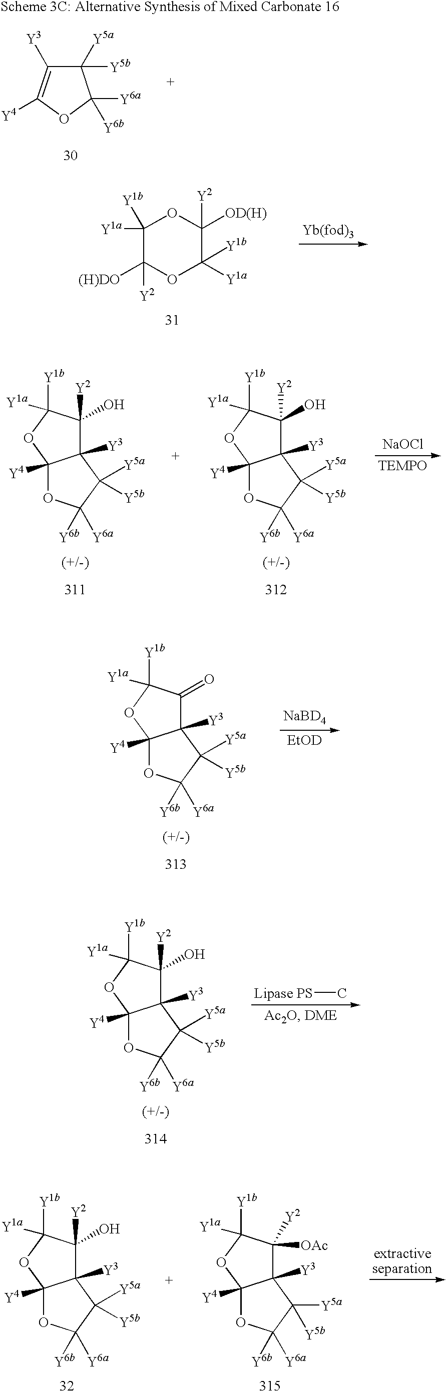

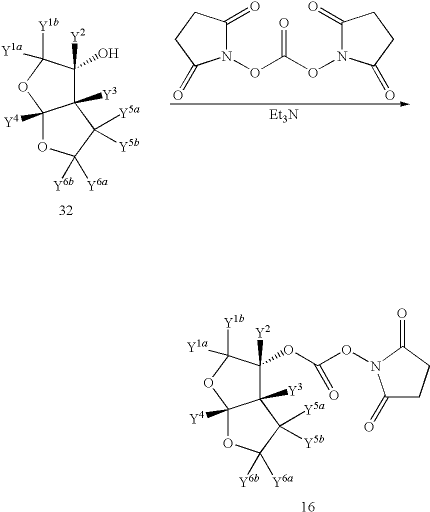

An alternative preparation of the appropriately deuterated analogs of intermediate 16 is shown in Scheme 3C. The appropriately deuterated dihydrofuran 30 is reacted with the deuterated glycolaldehyde dimer 31 in the presence of catalytic ytterbium tris(6,6,7,7,8,8,8-heptafluoro-2,2-dimethyl-3,5-octanedionate “Yb(fod)₃” to afford a diastereomeric mixture of the bicyclic alcohols 311 and 312. Subsequent oxidation of 311 and 312 with sodium hypochlorite and 2,2,6,6-tetramethylpiperidine-1-oxyl “TEMPO” gives the corresponding ketone 313 which is reduced with sodium borodeuteride in dl-ethanol to afford the racemic cis-bisfuran alcohol 314. Resolution of the racemic alcohol 314 with lipase PS—C amino I enzyme gives the resolved enantiomer of cis-bisfuran alcohol 32 and the antipode cis-bisfuran acetate 115. Via an extractive workup the undesired antipode acetate is extracted away into the organic layer, allowing the desired alcohol 32 to be isolated from the aqueous layer. Appropriately deuterated analogs of 32 can then be converted to the mixed carbonate 16 by reaction with disuccinimidyl carbonate and triethylamine in acetonitrile as described by Ghosh, A K et al., in J Org Chem, 2004, 69: 7822-7829.

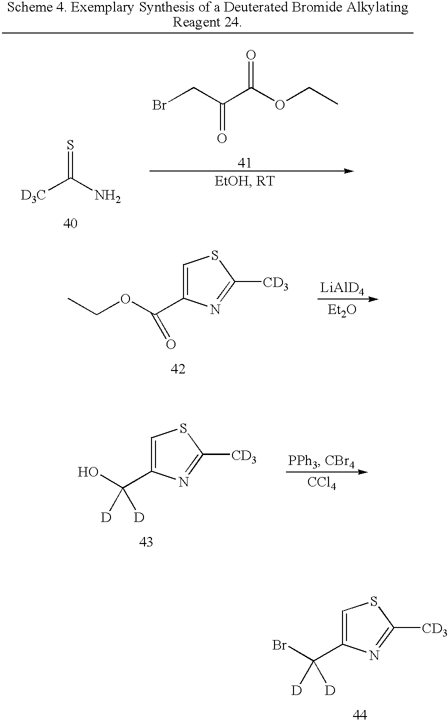

Scheme 4 depicts a synthesis of 2-d₃-methyl-5-d₂-bromomethylthiazole 44, which is an example of a deuterated alkylating reagent (reagent 24) useful in Scheme 2. Deuterated thioacetamide 40 is prepared as described by Anthoni, U et al, J Label Comp Radiopharm 1984, 21(4): 375-380 and then reacted with ethyl 3-bromo-2-oxopropanoate (41) to form thiazole carboxylate 42. Following the published procedure of Jung, J-C et al, J Org Chem 2004, 69(26):9269-9284, the thiazole 42 is then converted to the desired compound 44 as shown. Reduction of the ethyl ester 42 with LiAlD₄ provided the deuterated alcohol 43, which is then readily converted to 2-d₃-methyl-5-d₂-bromomethylthiazole 44 with PPh₃ and CBr₄ in CCl₄.

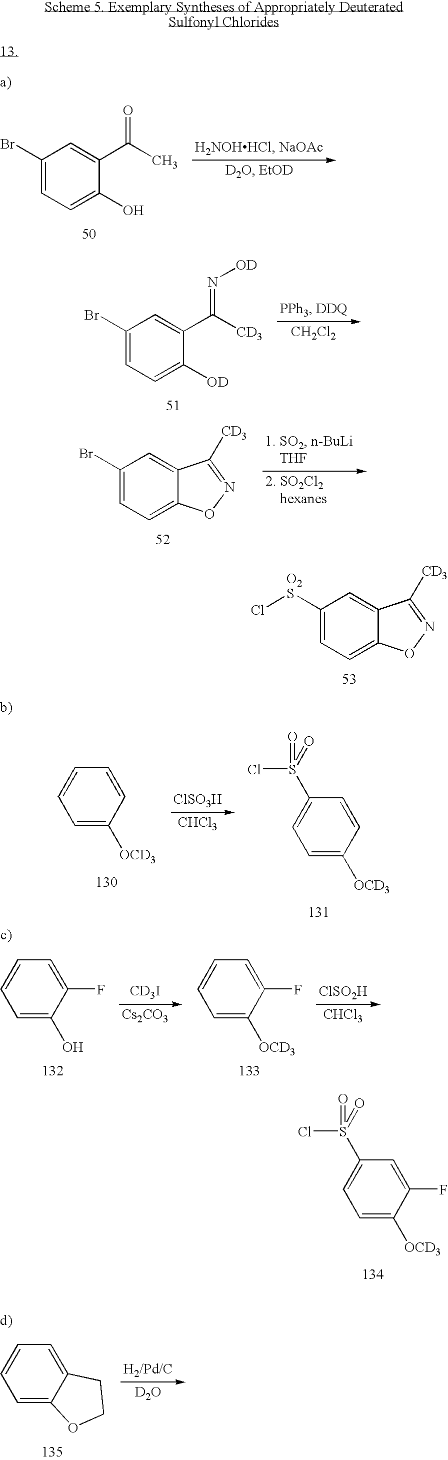

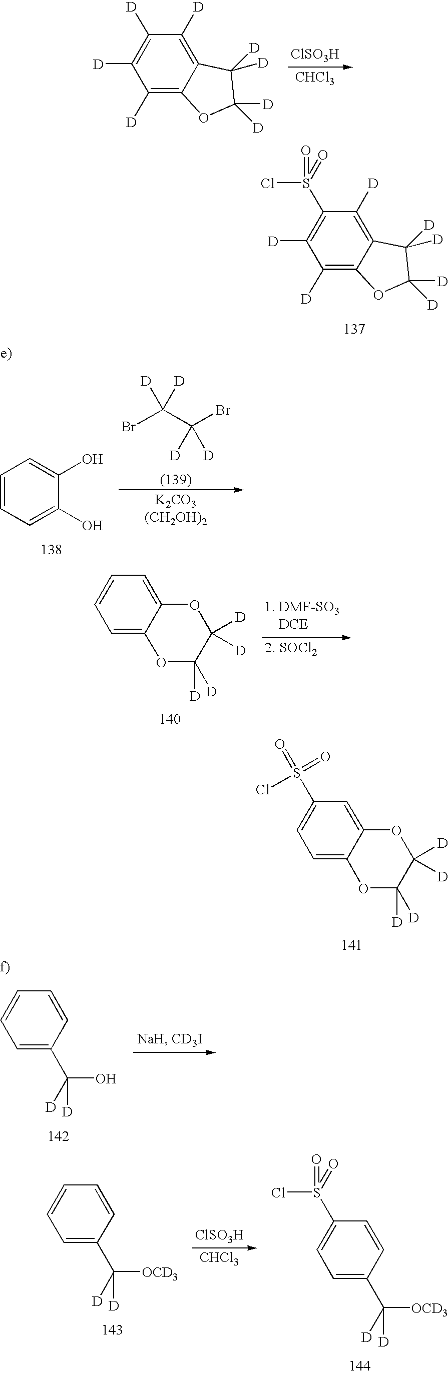

Scheme 5a) depicts the synthesis of 3-d₃-methylbenzo[d]isoxazole-5-sulfonyl chloride 53, which can be utilized as an appropriately deuterated sulfonyl chloride (reagent 13) in Scheme 1 or 2. Synthesis of the oxime is accomplished as described by Chandra, S et al, J Ind Chem Soc 2004, 81(3): 203-206 by reaction of acetophenone 50 with hydroxylamine HCl. The use of deuterated solvent in this reaction will yield the oxime 51 in which the exchangeable protons have been replaced by deuterium. This oxime is then cyclized with PPh₃ and DDQ to the benzisoxazole 52 as described by Iranpoor, N. et al, Tet Lett 2006, 47(47): 8247-8250. The bromide 52 is then converted to the sulfonyl chloride 53 according to the procedure of Thomas, P J et al, Syn Comm (1995), 25(18), 2813-2817.

Scheme 5b) depicts the synthesis of 4-trideuteromethoxybenzenesulfonyl chloride 131 by treatment of phenyl trideuteromethyl ether 130 with chlorosulfonic acid, analogously to the procedure described in J. Am. Chem. Soc. 1948, 70, 375-78.

Scheme 5c) depicts the synthesis of 3-fluoro-4-trideuteromethoxybenzene-1-sulfonyl chloride 134. o-fluorophenol 132 is treated with trideuteromethyl iodide to provide 133, which is then treated with chlorosulfonic acid to give 134.

Scheme 5d) depicts the synthesis of d₇-2,3-dihydrobenzofuran-5-sulfonyl chloride 137. 2,3-dihydrobenzofuran 135 is treated with hydrogen and Pd/C followed by D₂O to afford d₈-2,3-dihydrobenzofuran 136, which is then treated with chlorosulfonic acid to give 137. This may be accomplished in accordance with Tetrahedron, 62 (2006), 10954-61. Alternatively, 135 is treated with Raney Nickel to obtain 136 in accordance with the procedure described in Tetrahedron Lett. 1984, 25, 2507-08.

Scheme 5e) depicts the synthesis of 2,2,3,3-tetradeutero-2,3-dihydrobenzo[b][1,4]dioxine-6-sulfonyl chloride 141. Catechol 138 is treated with d₄-1,2-dibromoethane 139 in a manner analogous to J. Org. Chem. 1987, 52, 5616-21 to provide 2,2,3,3-deutero-2,3-dihydrobenzo[b][1,4]dioxine 140, which is then treated with DMF-SO₃/DCE followed by thionyl chloride in a manner analogous to the one described in European patent publication EP 583960 to give 141.

Scheme 5f) depicts the synthesis of 4-(dideutero(trideuteromethoxy)methyl)benzene-1-sulfonyl chloride 144. Dideutero(phenyl)methanol 142 is treated with NaH and trideuteromethyl iodide to provide (dideutero(trideuteromethoxy)methyl)benzene 143, which is then treated with chlorosulfonic acid to give 144. In addition to deuterated sulfonyl chlorides, such as the ones prepared as shown in exemplary schemes 5a-5e, the following sulfonyl chlorides are useful for the preparation of the compounds of the invention. All of the following sulfonyl chlorides are commercially available:

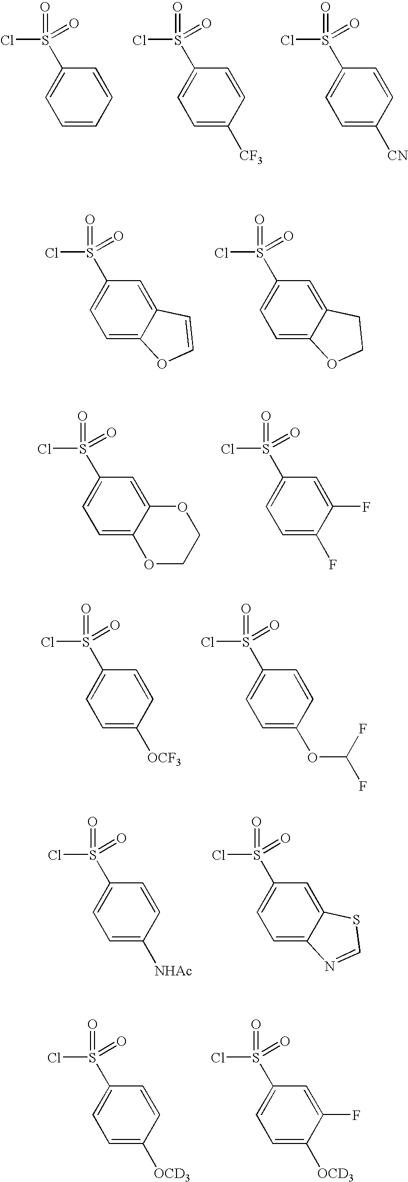

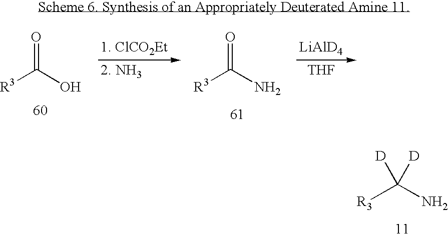

The appropriately deuterated amine 11 can be prepared as shown in Scheme 6. A deuterated carboxylic acid 60, such as commercially available isobutyric acid, is activated as the mixed anhydride with ethyl chloroformate and then reacted with ammonia to provide the amide 61 according to the general procedure for amide formation disclosed by Alvarado, C et al, Tet Lett 2007, 48: 603-607. Alternately, carbonyldiimidazole may be used in place of ethyl chloroformate. The amide 61 can be readily converted to the amine 11 by reduction with lithium aluminum deuteride as disclosed, for example, by Poehler, T et al, Eur J Med Chem 2007, 42: 175-197.

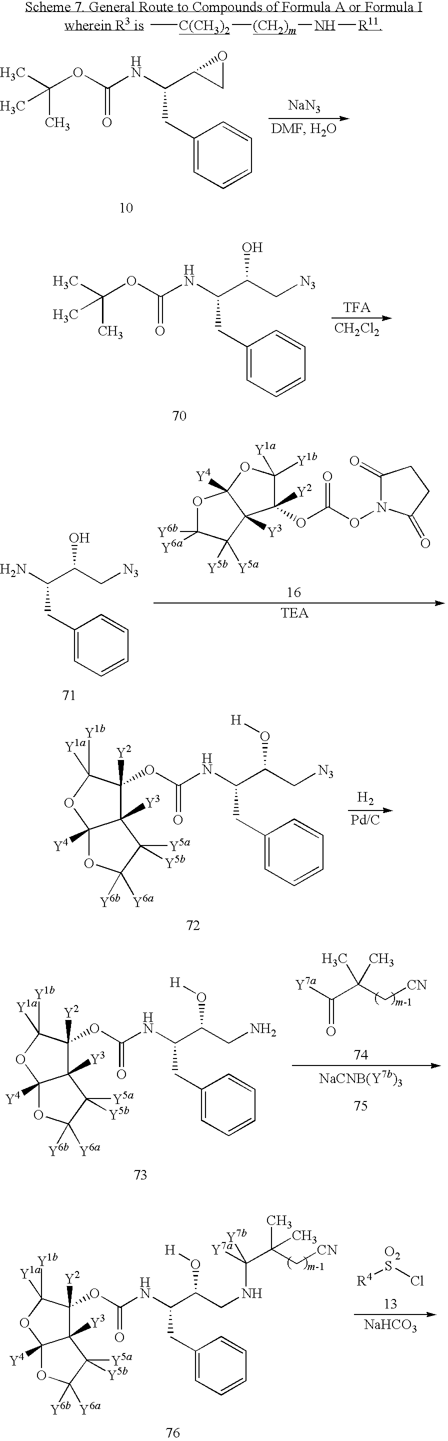

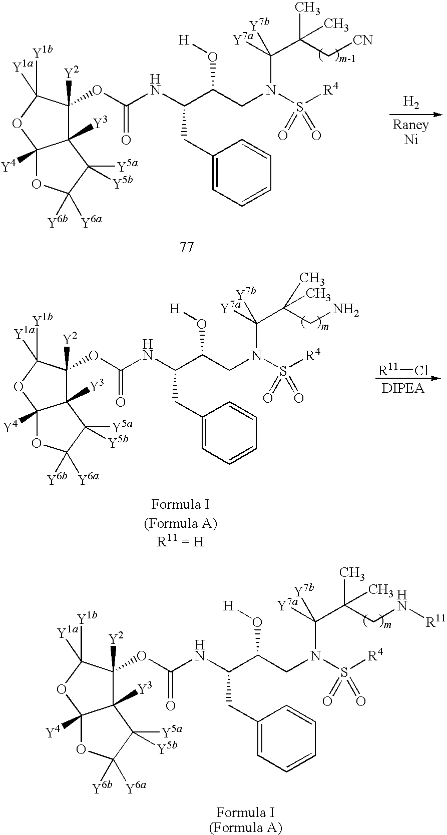

Compounds of the invention where R³ is —C(CH₃)₂—(CH₂)ₘ—NH—R¹¹ can be prepared as depicted in Scheme 7. Treatment of the commercially available chiral epoxide (10) with sodium azide according to the procedure described by Miller, J. F et al, Bioorg Med Chem Lett 2004, 14: 959-963 affords azido alcohol 70. Using the procedures described by Miller, removal of the N-tert-butoxycarbonyl group (Boc) group with trifluoroacetic acid (TFA) gives the amino alcohol 71 which is then reacted with the appropriately deuterated mixed carbonate 16 in the presence of triethylamine to give carbamate 72. Reduction the azido group is accomplished by treatment with hydrogen in the presence of a palladium on carbon catalyst to give amine 73. Reductive amination of amine 73 with the appropriately deuterated cyano-aldehyde (74) in the presence of either sodium cyanoborodeuteride or sodium cyanoborohydride (NaCNB(Y^{7b})₃) affords the appropriately deuterated amines 76. Sulfonylation of the resulting amine with the appropriately deuterated sulfonyl chloride 13 in the presence of NaHCO₃ provides sulfonamide 77. Reduction of the cyano group with hydrogen in the presence of a Raney nickel catalyst affords a compound of the invention having a primary amine (R¹¹═H). Subsequent N-alkylation of the primary amine with the appropriately deuterated chloride (R¹¹-Cl) affords the desired compounds of the invention wherein R¹¹ is other than hydrogen.

In the foregoing schemes, R⁴ (or R^{4′}) may be any specific R⁴ (or R^{4′}) disclosed herein. Schemes 8 and 10 illustrate the preparation of compounds of Formula A or Formula I for the case where R⁴ (or R^{4′}) is a substituted benzofuran or a substituted benzothiazole, respectively.

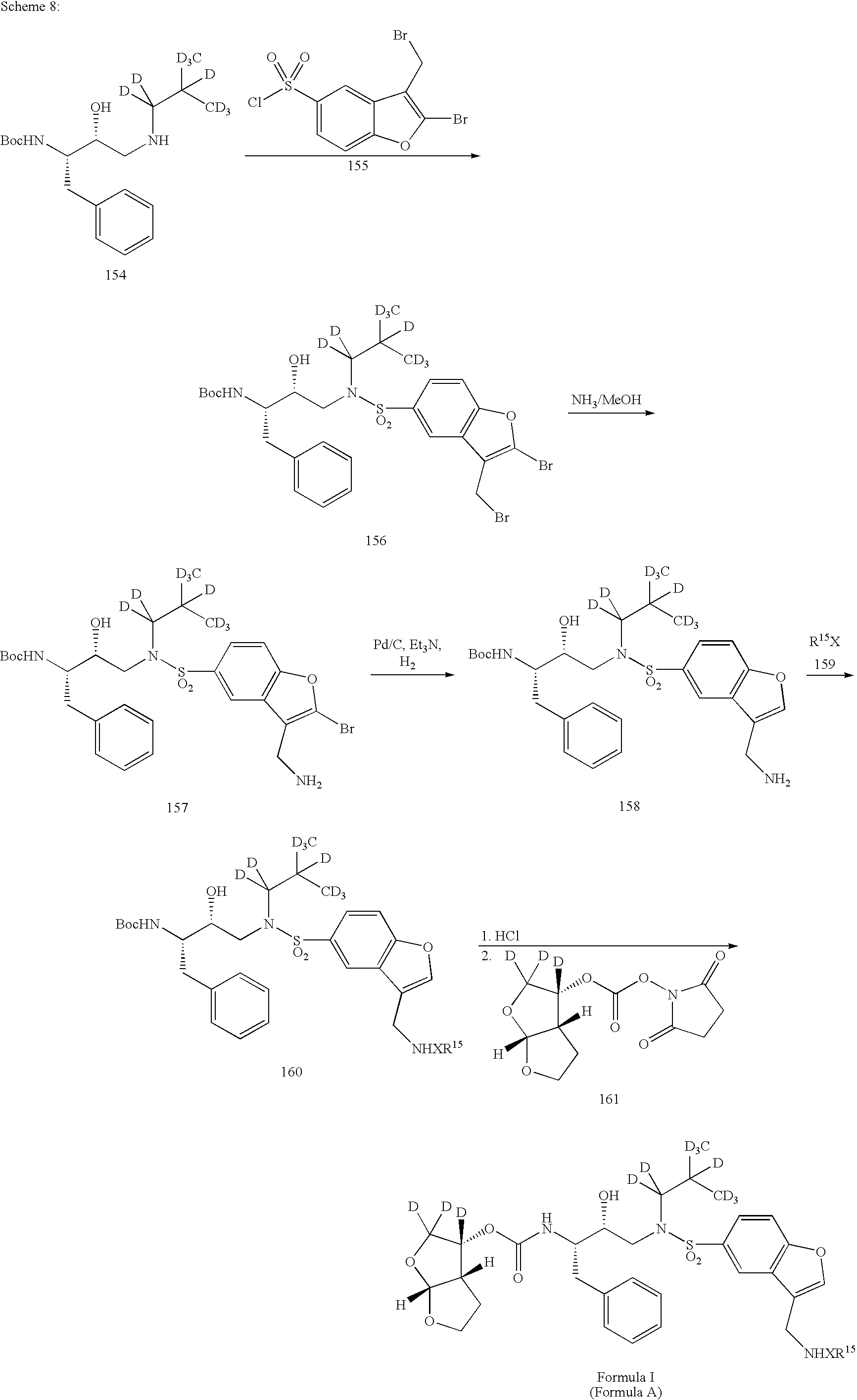

As an example, XR¹⁵ in Scheme 8 is —CO₂C₁₋₆ alkyl such as —CO₂C₂H₅. As shown in Scheme 8, 154 is sulfonylated with 155 (prepared as described in Scheme 9 below) to provide 156. Treatment of 156 with ammonia to give 157 followed by reductive dehalogenation with H₂ and Pd/C affords 158. Treatment of 158 with 159 gives 160. Removal of the protecting group of 160 followed by reaction with 161 provides a compound of Formula A or Formula I.

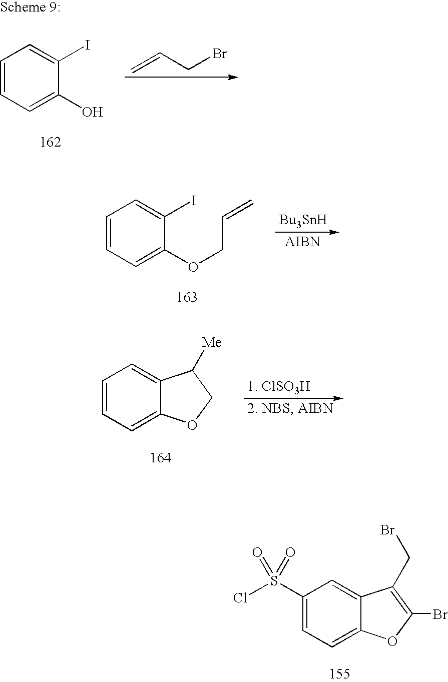

As shown in Scheme 9, 162 is allylated to provide 163 which upon treatment with tributyltin hydride and AIBN cyclizes to give 164. 164 is then treated with chlorosulfonic acid and subsequently with N-bromosuccinimide and AIBN to give 155.

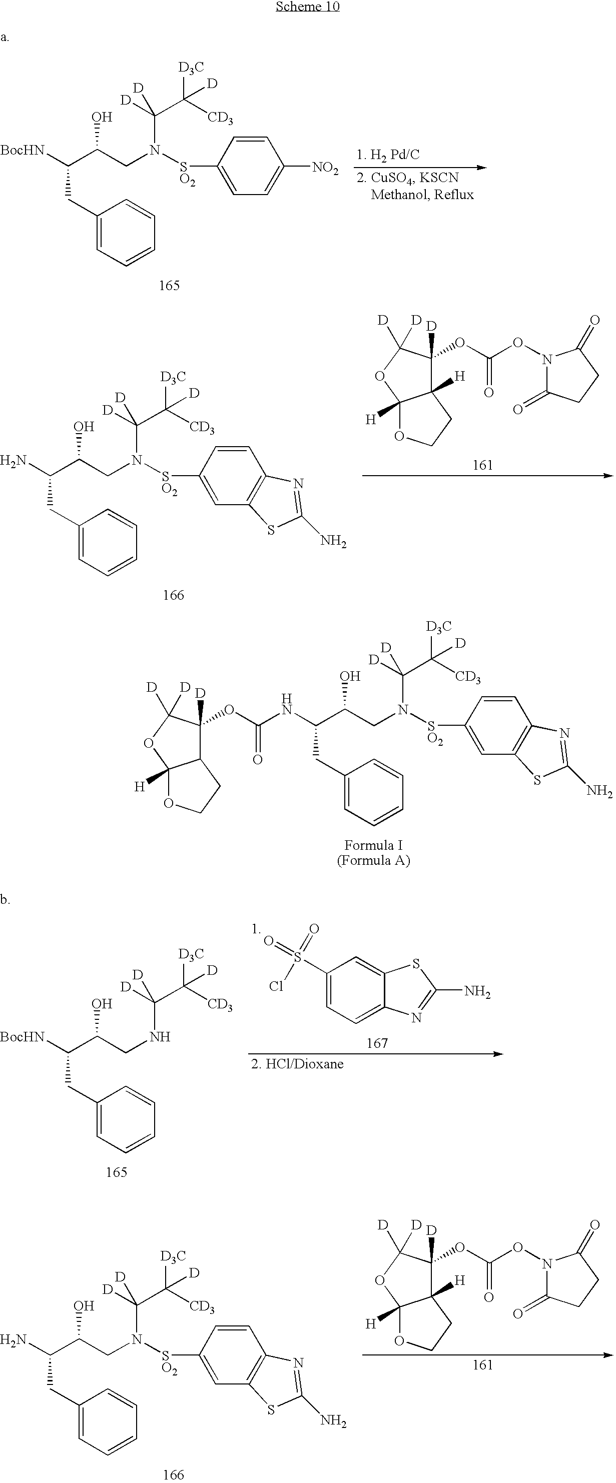

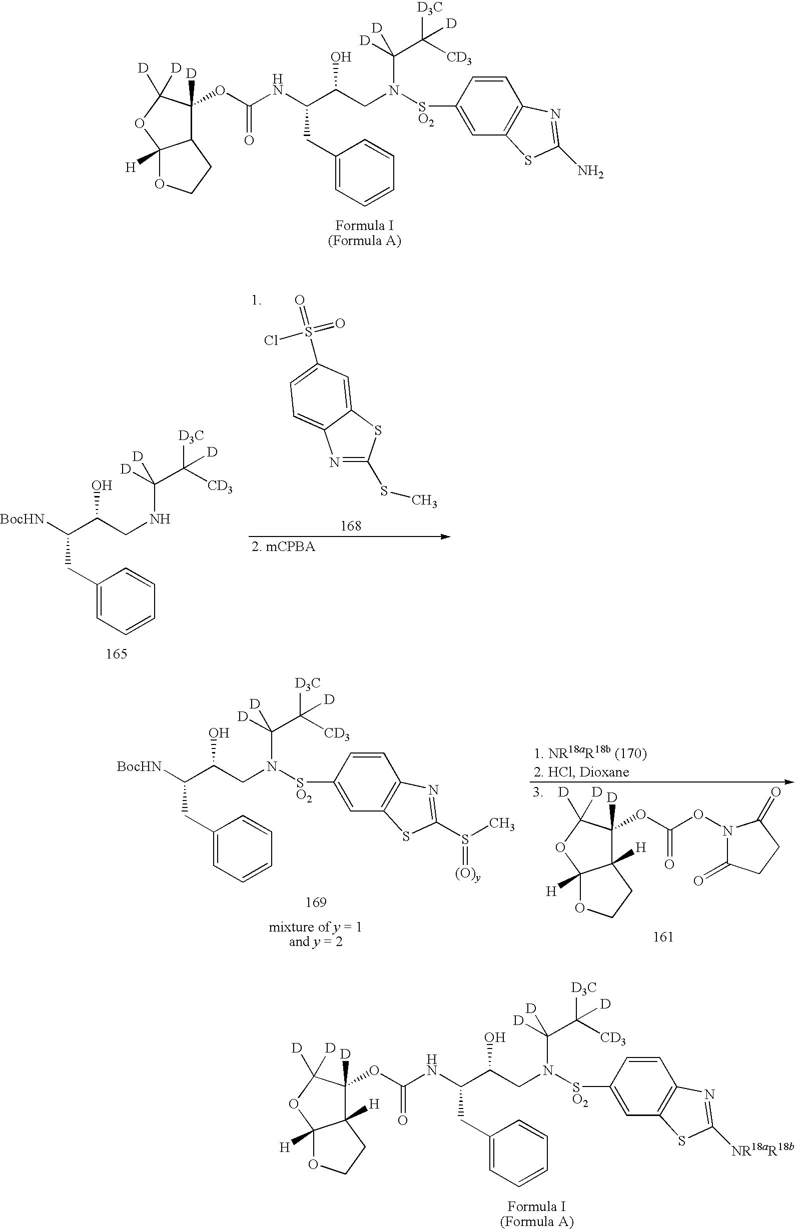

Scheme 10 provides three alternatives for the preparation of compounds of formula I where R⁴ is a substituted benzothiazole. As shown in Scheme 10a), 165 is treated with H₂ and Pd/C followed by cupric sulfate and KSCN to give 166, which is then treated with 161 to afford a compound of Formula A or Formula I. Alternatively, as shown in Scheme 10b), 165 is sulfonylated with 167 and the resulting sulfonamide is treated with HCl to give 166, which is converted to a compound of Formula A or Formula I in the same manner as shown in Scheme 10a. Alternatively, as shown in Scheme 10c), 165 is sulfonylated with 168 and the resulting thioether is oxidized with mCPBA to give 169. Treatment of 169 with 170 is followed by treatment with HCl and reaction with 161 to aive a compound of Formula A or Formula I.

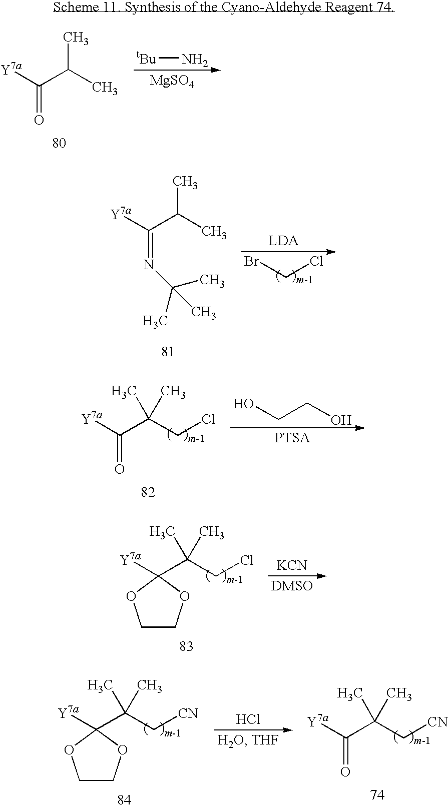

The appropriately deuterated cyano-aldehyde 74 can be prepared as shown in Scheme 11. An appropriately deuterated aldehyde 80, such as commercially available isobutyraldehyde, is treated with tert-butylamine in the presence of magnesium sulfate according to the procedure described by De Kimpe, N et al, Tetrahedron 1997, 53: 10803-10816 to afford imine 81. Treatment of the aza-enolate (generated by treatment of the imines with lithium diisopropylamide) with the appropriately deuterated bromochloroalkane according to the procedure described by Miller, J. F et al, Bioorg Med Chem Lett 2004, 14: 959-963 affords the appropriately deuterated chloro-aldehyde 82. Using the procedures described by Miller, treatment of the aldehyde with ethylene glycol in the presence of p-toluenesulfonic acid (PTSA) provides chloro-acetal 83 which undergoes nucleophilic displacement upon treatment with potassium cyanide to afford cyano-acetal 84. Finally, hydrolysis of the acetal with aqueous hydrochloric acid affords the desired deuterated cyano-aldehyde 74.

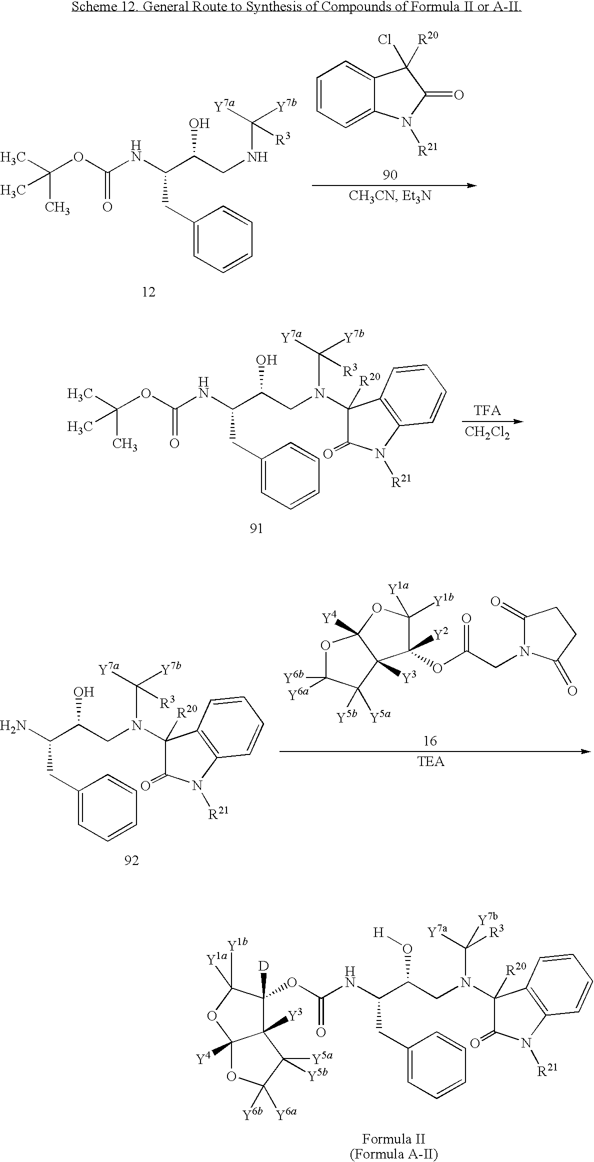

Compounds of Formula II or A-II can be prepared as depicted in Scheme 12. Treatment of secondary amine 12 with the appropriately deuterated oxyindole derivative (90) according to the procedure described by Ghosh, A K et al, Bioorg Med Chem Lett 2006, 16: 1869-1873 affords amino alcohol 91. Using the procedures described by Ghosh, removal of the N-tert-butoxycarbonyl group (Boc) group with trifluoroacetic acid (TFA) gives the amino alcohol 92 which is then reacted with the appropriately deuterated mixed carbonate 16 in the presence of triethylamine to afford the desired compounds of Formula II or A-II.

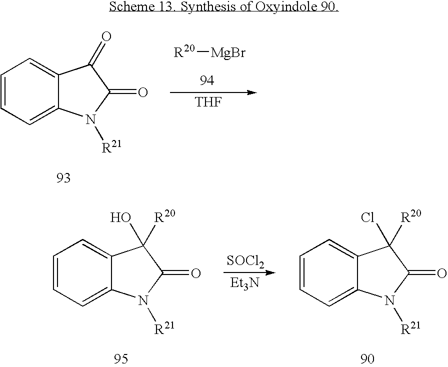

The appropriately deuterated oxyindole (90) can be prepared as shown in Scheme 13. Treatment of a commercially available isatin (93) with the appropriately deuterated Grignard reagent 94 according to the procedure described by Ghosh, A K et al, Bioorg Med Chem Lett 2006, 16: 1869-1873 gives the tertiary alcohol 95. Using the procedures of Ghosh, treatment of alcohol 95 with thionyl chloride in the presence of triethylamine affords the desired oxyindole 90.

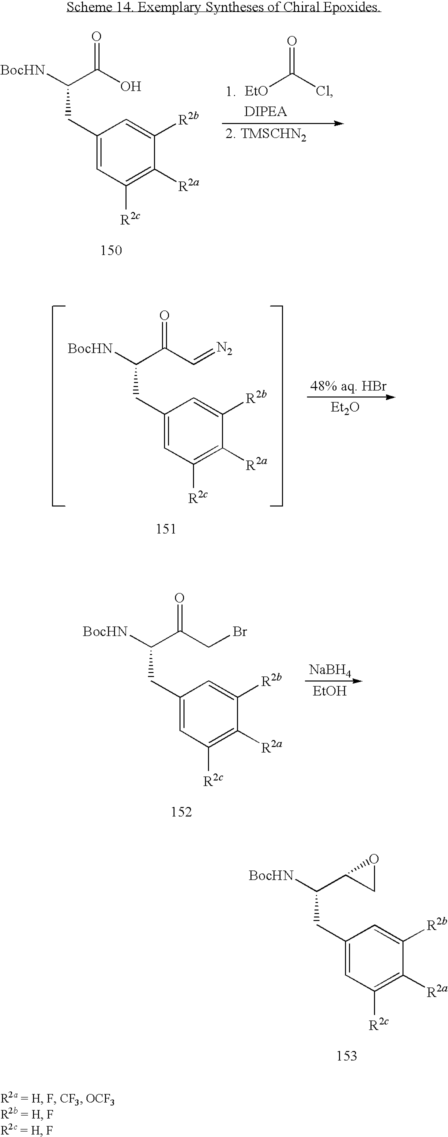

Scheme 14 shows the preparation of chiral epoxides that may be used instead of chiral epoxides 10 and 20 in Schemes 1 and 2, respectively. Scheme 11 may be suitable, for example, where R^{2a} is H, F, CF₃ or OCF₃ In a manner analogous to Rotella, D. P. Tetrahedron Letters 1995, 36, 5453-5456, chiral N-protected amino acid 150 is treated with ethyl chloroformate followed by trimethylsilyldiazomethane to provide 151, which is treated with hydrobromic acid to afford α-bromoketone 152. Treatment with sodium borohydride gives chiral epoxide 153. Examples of chiral epoxides that may be prepared in accordance with Scheme 11 include:

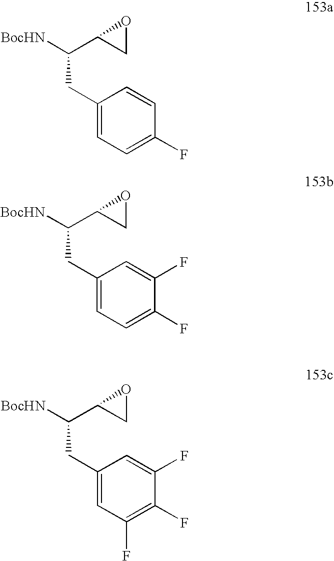

where in 153a-c, Boc represents the group t-butoxycarbonyl.

Examples of chiral N-protected amino acids include the following compounds:

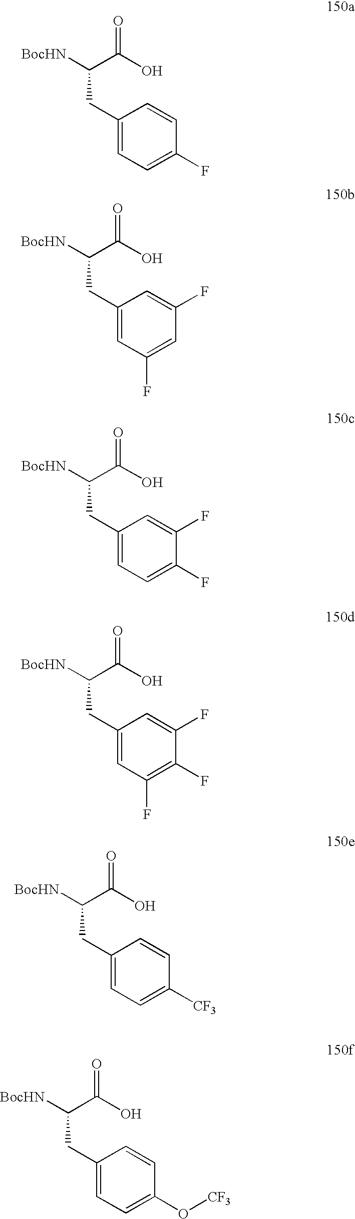

where in 150a-f, Boc represents the group t-butoxycarbonyl.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free pharmaceutical compositions comprising an effective amount of a compound of Formula I (e.g., including any of the formulae herein), or a pharmaceutically acceptable salt of said compound; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as darunavir. Such agents include those indicated as being useful in combination with darunavir, including but not limited to, those described in WO 2003049746, WO 2005027855, and WO 2006005720.

In one embodiment the second therapeutic agent is an agent useful in the treatment or prevention of a disease including, but not limited to, (HIV) infection and malaria.

In one embodiment, the second therapeutic agent is selected from other anti-retroviral agents or a pharmacokinetic enhancing agent including, but not limited to, a second HIV protease inhibitor (e.g., amprenavir, fosamprenavir, tipranavir, indinavir, saquinavir, lopinavir, ritonavir, atazanavir, or nelfinavir), a non-nucleoside reverse transcriptase inhibitor (“NNRTI”) (e.g., UK-453061, GSK 2248761, etravirine, delavirdine, efavirenz, nevirapine, or rilpivirine), a nucleoside/nucleotide reverse transcriptase inhibitor (“NRTI”) (e.g., zidovudine, lamivudine, emtricitabine, tenofovir disoproxil fumarate, didanosine, stavudine, abacavir, racivir, amdoxovir, apricitabine, entecavir, adefovir or elvucitabine) a CCR5 antagonist (e.g., PF-232798; GSK 706769, enfuvirtide, maraviroc, vicriviroc, PRO140, or TNX-355), an integrase inhibitor (e.g., GSK 1349572, raltegravir, or elvitegravir), an immune based antiretroviral agent (e.g., immunitin, proleukin, remune, BAY 50-4798 or IR103), a viral maturation inhibitor (e.g., bevirimat), a cellular inhibitor (e.g., droxia or hydroxyurea), or a pharmacokinetic enhancing agent (e.g., ritonavir, GS 9350; PF-03716539) combinations of two or more of the above.

In a more specific embodiment, the second therapeutic agent is selected from efavirenz, didanosine, tenofovir disoproxil, nelfinavir mesylate, raltegravir, saquinavir, lopinavir, nevirapine, emtricitabine, abacavir, lamivudine, zidovudine, maraviroc, stavudine, darunavir, fosamprenavir, vicriviroc, GSK 1349572, UK-453061, PF-03716539, etravirine, pharmaceutically acceptable salts of any of the foregoing, and combinations thereof.

In yet another specific embodiment, the second therapeutic agent is ritonavir.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., Cancer Chemother. Rep, 1966, 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 1 mg to about 6000 mg per treatment. In more specific embodiments the range is from about 10 mg to 3000 mg, or from about 20 mg to 1200 mg, or most specifically from about 100 mg to 600 mg per treatment. Treatment typically is administered twice daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for darunavir.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of inhibiting the activity of HIV protease in an infected cell, comprising contacting such cell with one or more compounds of Formulas A, A-1, I, Id, Ie, If, Ig, II and A-II herein.

According to another embodiment, the invention provides a method of treating a disease that is beneficially treated by darunavir in a patient in need thereof comprising the step of administering to said patient an effective amount of a compound or a composition of this invention. Such diseases are well known in the art and are disclosed in, but not limited to the following patents and published applications: WO 1994004492, WO 1995006030, U.S. Pat. No. 6,335,460, and WO 2005027855. Such diseases include, but are not limited to, human immunodeficiency virus (HIV) infection and malaria.

In one particular embodiment, the method of this invention is used to treat HIV infection in a patient in need thereof.

Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with darunavir. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include co-administering to a patient in need thereof a compound of Formulas A, A-1, I, Id, Ie, If, Ig, II and A-II and a second therapeutic agent selected from a second HIV protease inhibitor (e.g., amprenavir, fosamprenavir, tipranavir, indinavir, saquinavir, lopinavir, ritonavir, atazanavir, or nelfinavir), a non-nucleoside reverse transcriptase inhibitor (“NNRTI”) (e.g., UK-453061, GSK 2248761, etravirine, delavirdine, efavirenz, nevirapine, or rilpivirine), a nucleoside/nucleotide reverse transcriptase inhibitor (“NRTI”) (e.g., zidovudine, lamivudine, emtricitabine, tenofovir disoproxil fumarate, didanosine, stavudine, abacavir, racivir, amdoxovir, apricitabine, entecavir, adefovir or elvucitabine) a CCR5 antagonist (e.g., PF-232798; GSK 706769, enfuvirtide, maraviroc, vicriviroc, PRO 140, or TNX-355), an integrase inhibitor (e.g., GSK 1349572, raltegravir, or elvitegravir), an immune based antiretroviral agent (e.g., immunitin, proleukin, remune, BAY 50-4798 or IR103), a viral maturation inhibitor (e.g., bevirimat), a cellular inhibitor (e.g., droxia or hydroxyurea), or a pharmacokinetic enhancing agent (e.g., ritonavir, GS 9350; PF-03716539) combinations of two or more of the above.

In a more specific embodiment, the combination therapies of this invention include co-administering to a patient in need thereof a compound of Formulas A, A-1, I, Id, Ie, If, Ig, II and A-II and a second therapeutic agent selected from efavirenz, didanosine, tenofovir disoproxil, nelfinavir mesylate, raltegravir, saquinavir, lopinavir, nevirapine, emtricitabine, abacavir, lamivudine, zidovudine, maraviroc, stavudine, darunavir, fosamprenavir, vicriviroc, GSK 1349572, UK-453061, PF-03716539, etravirine, pharmaceutically acceptable salts of any of the foregoing, and combinations thereof.

In another specific embodiment, the combination therapy of this invention comprises the step of co-administering to a patient in need thereof a compound of Formulas A, A-1, I, Id, Ie, If, Ig, II and A-II and ritonavir.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formulas A, A-1, I, Id, Ie, If, Ig, II and A-II alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formulas A, A-1, I, Id, Ie, If, Ig, II and A-II for use in the treatment or prevention in a patient of a disease, disorder or symptom thereof delineated herein.

### EXAMPLES

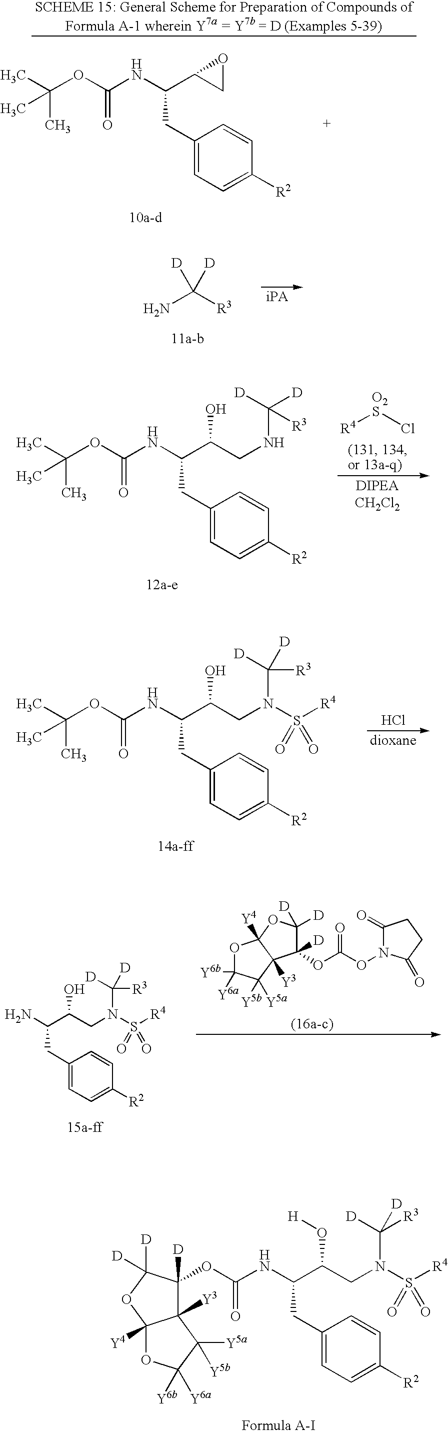

### Example 1

Synthesis of Intermediates 10b-10d. Intermediates 10b, 10c, and 10d were prepared as shown in Scheme 16 and as described in Examples 1A, 1B, and 1C below.

| Intermediate | R² | Example No. |
|---|---|---|
| 10a | H | commercially available |
| 10b | OCD₃ | 1A |
| 10c | OCD(CD₃)₂ | 1B |
| 10d | OCD₂Ph | 1C |

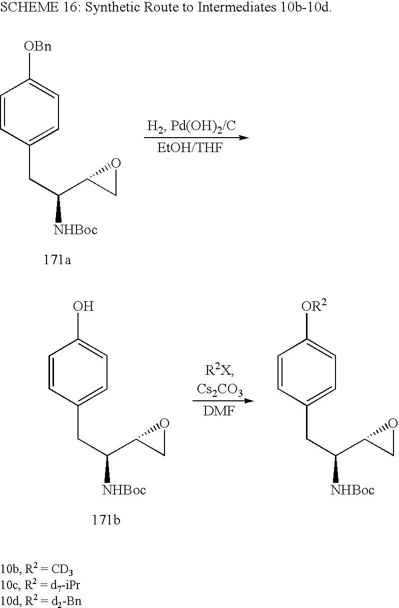

### Example 1A

Synthesis of erythro-N-Boc-O-(methyl-d3)-L-tyrosine epoxide (10b). To a solution of erythro-N-Boc-O-benzyl-L-tyrosine epoxide 171a (5.00 g, 13.5 mmol, available from Bepharm Product List) in ethanol (120 mL) and THF (30 mL) was added Pd(OH)₂/C (50 wt % H₂O, 500 mg). The reaction was flushed several times with nitrogen then stirred under an atmosphere of hydrogen until starting material was no longer observed by LCMS (˜4 hours). At this time, the reaction was flushed repeatedly with nitrogen and filtered through Celite®. The filter cake was then washed with 20% MeOH/CH₂Cl₂ (3×20 mL) and the combined washings were concentrated under reduced pressure. The resulting residue was purified by silica gel column chromatography on an ISCO system (0-10% MeOH/CH₂Cl₂) to afford 171b (3.42 g, 91% yield) as a white solid. MS (ESI) 280.1 [(M+H)⁺].

To a solution of 171b (1.00 g, 3.58 mmol) in DMF (20 mL) was added Cs₂CO₃ (3.50 g, 10.7 mmol) followed by iodo methane-d3 (445 μL, 7.16 mmol; Aldrich, 99.5 atom % D). The reaction stirred for 15 hours then was diluted with ether and filtered through Celite®. The filter cake was then washed with ether (2×100 mL) and water (3×100 mL). The organic layer was separated, dried (MgSO₄), filtered, and concentrated under reduced pressure to afford 10b (1.07 g, 100% yield) as a white solid. MS (ESI) 297.2 [(M+H)⁺].

### Example 1B

Synthesis of erythro-N-Boc-O-(isopropyl-d7)-L-tyrosine epoxide (10c). Intermediate 10c was prepared according to the procedure for 10b. 2-iodo propane-d7 (Aldrich, 98 atom % D) was used in lieu of iodo methane-d3. MS (ESI) 329.3 [(M+H)⁺].

### Example 1C

erythro-N-Boc-O-benzyl-L-tyrosine epoxide-d2 (10d). Intermediate 10d was prepared according to the procedure for 10b. Benzyl bromide-d2 (CDN Isotopes, 98.8 atom % D) was used in lieu of iodo methane-d3. MS (ESI) 372.3 [(M+H)⁺].

### Example 2

Synthesis of Intermediate 11b. Intermediate 11b was prepared as shown in Scheme 17 and as described below.

| Intermediate | R³ | Example No. |
|---|---|---|
| 11a | CD(CD₃)₂ | commercially available |
| 11b | Cyclopentyl-d₉ | 2 |

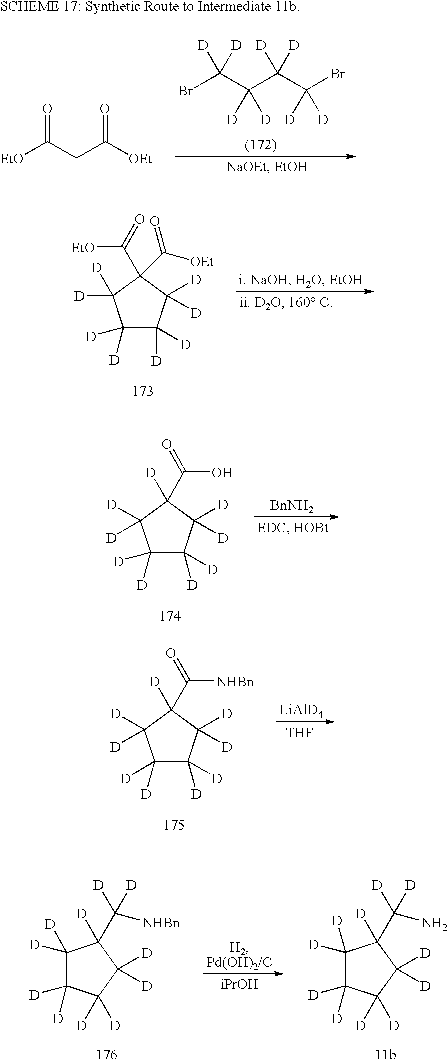

Step 1. Diethyl(Cyclopentane-d8)-1,1-dicarboxylate (173): To a solution of diethylmalonate (3.20 mL, 21.3 mmol) in ethanol (20 mL) was added 1,4-dibromobutane-d8, 172 (5.00 g, 22.3 mmol; CDN Isotopes, 99.6 atom % D) followed by dropwise addition of sodium ethoxide (16.0 mL, 42.5 mmol, 21 wt % in ethanol). The reaction was heated to 100° C. and stirred for 2 hours then cooled to room temperature, diluted with water and concentrated under reduced pressure to remove ethanol. The resulting aqueous solution was extracted with EtOAc (3×50 mL) and the organic layers were combined, dried (MgSO₄), filtered and concentrated under reduced pressure to afford 173 (2.28 g, 48%). MS (ESI) 223.1 [(M+H)⁺].

Step 2. (Cyclopentane-d9)carboxylic acid (174). To a solution of 173 (2.28 g, 10.3 mmol) in ethanol (5.4 mL) was added 5N NaOH (4.70 mL, 23.6 mmol). The reaction was then stirred at reflux for 2 hours, cooled to room temperature and concentrated under vacuum to remove ethanol. The resulting aqueous solution was acidified to pH 2 with 6N HCl then extracted with diethyl ether (3×20 mL). The organic layers were combined, dried (Na₂SO₄), filtered and concentrated. The resulting diacid (1.6 g, 9.62 mmol) was diluted with D₂O (40 mL; Cambridge Isotope Laboratories, 99.8 atom % D) in a pressure flask and sealed. The reaction stirred at 160° C. for 15 hours then was cooled to room temperature and diluted with 1N HCl. The aqueous solution was then extracted with EtOAc (3×25 mL) and the organic layers were combined, washed with brine, dried (Na₂SO₄), filtered and concentrated under reduced pressure to afford 174 (1.14 g, 90%-2 steps). MS (ESI) 122.2 [(M−H)⁻].

Step 3. N-Benzyl-(cyclopentane-d9)carboxamide (175). To a solution of acid 174 (1.14 g, 9.25 mmol) in CH₂Cl₂ (30 mL) at 0° C. was added benzylamine (1.21 mL, 11.1 mmol), EDC (2.66 g, 13.9 mmol), HOBt (1.87 g, 13.9 mmol), and NMM (4.10 mL, 37.0 mmol). The reaction stirred at room temperature for 15 hours then concentrated under reduced pressure. The resulting residue was diluted with 1N HCl and extracted with EtOAc (3×25 mL). The combined organic layers were washed sequentially with sat NaHCO₃, 1N HCl, and brine then dried (MgSO₄), filtered and concentrated under reduced pressure to afford 175 (1.90 g, 96%). MS (ESI) 213.3 [(M+H)⁺].

Step 4. N-benzyl-(cyclopentanemethyl-d11)amine (176). LiAlD₄ (0.750 g, 17.8 mmol; Cambridge Isotope Laboratories, 98 atom % D) was added portionwise to a solution of 175 (1.89 g, 8.91 mmol) in THF (36 mL) at 0° C. The reaction stirred at reflux for 15 hours then cooled to 0° C. and quenched via sequential dropwise addition of 0.75 mL water, 0.75 mL 15% NaOH, and 2.25 mL water. After stirring for 30 minutes the reaction was filtered through Celite® rinsing with diethyl ether. The filtrate was then concentrated under reduced pressure to afford 176 (1.60 g, 90%). MS (ESI) 201.2 [(M+H)⁺].

Step 5. (Cyclopentanemethyl-d11)amine (11b): 176 (1.60 g, 7.99 mmol) was dissolved in 2-propanol (13 mL) and the flask was evacuated with nitrogen several times. Pd(OH)₂/C (50% wet, 160 mg) was then added and the flask was again evacuated with nitrogen several times. The reaction was purged with H₂ and stirred under an atmosphere of H₂ for 15 hours. The vessel was then purged with nitrogen and the contents were filtered through Celite® rinsing with 2-propanol (7 mL) to afford 11b (879 mg, 100%) as a 0.4M solution in 2-propanol. MS (ESI) 111.2 [(M+H)⁺].

### Example 3

Synthesis of Intermediates 131, 134, 13m and 13n. Intermediates were prepared as shown in Schemes 18-20 and as described below.

| Intermediate | R⁴ | Example No. |
|---|---|---|
| 13a | 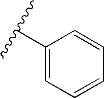 | commercially available |
| 131 | 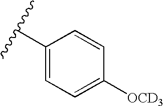 | 3A |
| 134 | 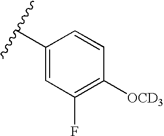 | 3B |
| 13b | 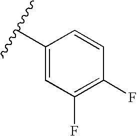 | commercially available |
| 13c | 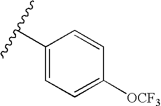 | commercially available |
| 13d | 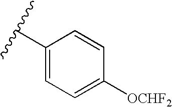 | commercially available |
| 13e | 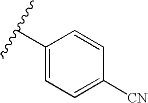 | commercially available |
| 13f | 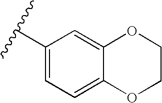 | commercially available |
| 13g | 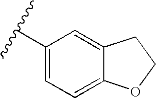 | commercially available |
| 13h | 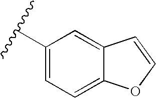 | commercially available |
| 13i | 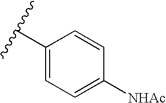 | commercially available |
| 13j | 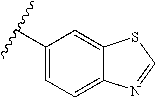 | commercially available |
| 13k | 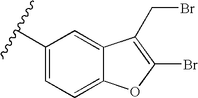 | Prepared as described in WO2005/110428 |
| 13-l | 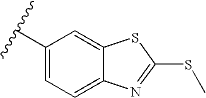 | Prepared as described in J. Med. Chem., 2005, 48: 1967. |
| 13m | 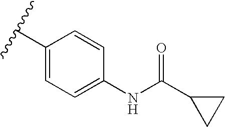 | 3C |
| 13n | 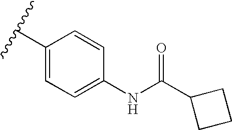 | 3D |
| 13o | 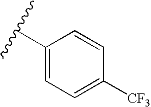 | commercially available |
| 13p | 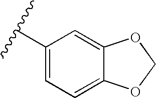 | commercially available |
| 13q | 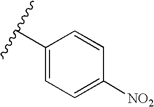 | commercially available |

### Example 3A

Synthesis of 4-(methoxy-d3)benzenesulfonyl chloride (131). Intermediate 131 was prepared as shown in Scheme 18.

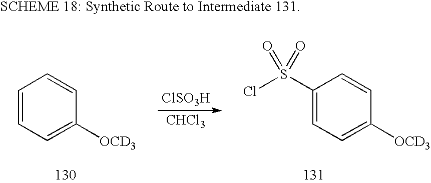

To a stirred solution of anisole-d3, 130 (1.00 mL, 9.01 mmol; CDN Isotopes, 99.8 atom % D) in CHCl₃ (3.75 mL) at −5° C. was added chlorosulfonic acid (1.20 mL, 18.0 mmol) dropwise. The reaction stirred at room temperature for 30 minutes then was poured over ice water and extracted with CHCl₃ (3×25 mL). The organic layer was dried (MgSO₄), filtered through Celite®, and concentrated under reduced pressure to afford 131 (461 mg, 24%). MS (ESI) sulfonic acid ion observed: 190.1 [(M−H)⁻].

### Example 3B

Synthesis of 3-Fluoro-4-(methoxy-d3)benzenesulfonyl chloride (134). Intermediate 134 was prepared as shown in Scheme 19.

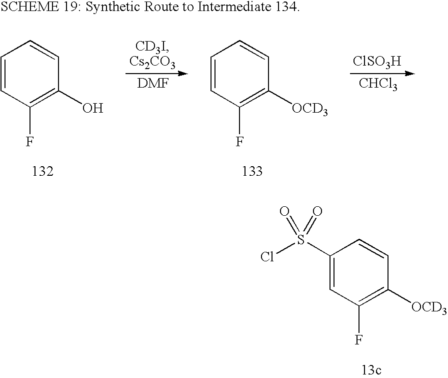

To a stirred solution of 2-fluorophenol, 132 (1.24 mL, 13.4 mmol) in DMF (60 mL) at room temperature was added cesium carbonate (13.1 g, 40.1 mmol) followed by iodo methane-d3 (1.66 mL, 26.8 mmol; Aldrich, 99.5 atom % D). The reaction stirred at room temperature for 72 hours then was diluted with diethyl ether (300 mL) and filtered through Celite®. The resulting solution was washed with water (3×200 mL) and the organic layer was dried (MgSO₄), filtered through Celite® and concentrated under reduced pressure to afford 2-fluoroanisole-d3, 133 as a yellow oil (910 mg. 53%).

To a stirred solution of 2-fluoroanisole-d3 (910 mg, 7.05 mmol) in CHCl₃ (3.00 mL) at −5° C. was added chlorosulfonic acid (942 μL, 14.1 mmol) dropwise. The reaction stirred at room temperature for 30 minutes then was poured over ice water and extracted with CHCl₃ (3×25 mL). The organic layer was dried (MgSO₄), filtered through Celite®, and concentrated under reduced pressure to afford 134 (1.04 g, 65%). MS (ESI) sulfonic acid ion observed: 208.1 [(M−H)⁻].

### Example 3C

Synthesis of 3-Fluoro-4-(methoxy-d3)benzenesulfonyl chloride (13m). Intermediate 13m was prepared as shown in Scheme 20.

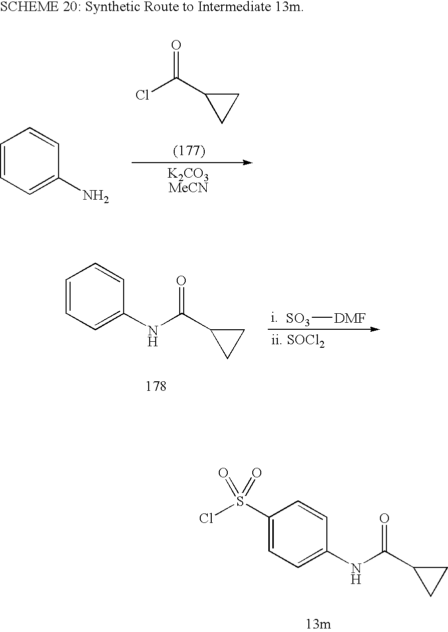

To a stirred solution of aniline (1.96 mL, 21.5 mmol) in acetonitrile (110 mL) at room temperature was added potassium carbonate (3.57 g, 25.8 mmol) followed by cyclopropane carbonyl chloride, 177 (1.97 mL, 21.5 mmol). The reaction stirred at room temperature for 2 hours then was concentrated under reduced pressure. The resulting residue was diluted with water (100 mL) and extracted with CH₂Cl₂ (3×50 mL). The organic layers were combined, washed with water (3×100 mL), dried (MgSO₄), filtered through Celite® and concentrated under reduced pressure. The material was recrystallized from EtOAc/hexanes to afford N-phenylcyclopropanecarboxamide, 178 as a white crystalline solid (1.66 g, 48%).

To a stirred solution of N-phenylcyclopropanecarboxamide (0.500 g, 3.10 mmol) in DCE was added SO₃-DMF (0.523 g, 3.41 mmol). The reaction was heated to 85° C. for 2 hours then was allowed to cool to room temperature. Thionyl chloride (271 μL, 3.72 mmol) was added dropwise and the reaction stirred at 75° C. for 1 hour. The reaction was then cooled to room temperature, diluted with CH₂Cl₂ (100 mL) and washed with cold water (5×50 mL). The organic layer was then dried (MgSO₄), filtered through Celite® and concentrated under reduced pressure. The resulting material was recrystallized from EtOAc/hexanes to afford 13m as a pale yellow solid (483 mg, 60%). MS (ESI) 260.0 [(M+H)⁺].

### Example 3D

Synthesis of 4-(Cyclobutanecarboxamido)benzene-1-sulfonyl chloride (13n).

Intermediate 13n was prepared as shown in Example 3C using cyclobutane carbonyl chloride in place of cyclopropane carbonyl chloride. MS (ESI) 274.0 [(M+H)⁺].

### Example 4A

Synthesis of 2,5-Dioxopyrrolidin-1-yl-(3R,3aS,6aR)-2,2,3-d₃-hexahydro furo[2,3-b]furan-3-yl-carbonate (16a). Intermediate 16a was prepared as shown in Scheme 21 and as described below.

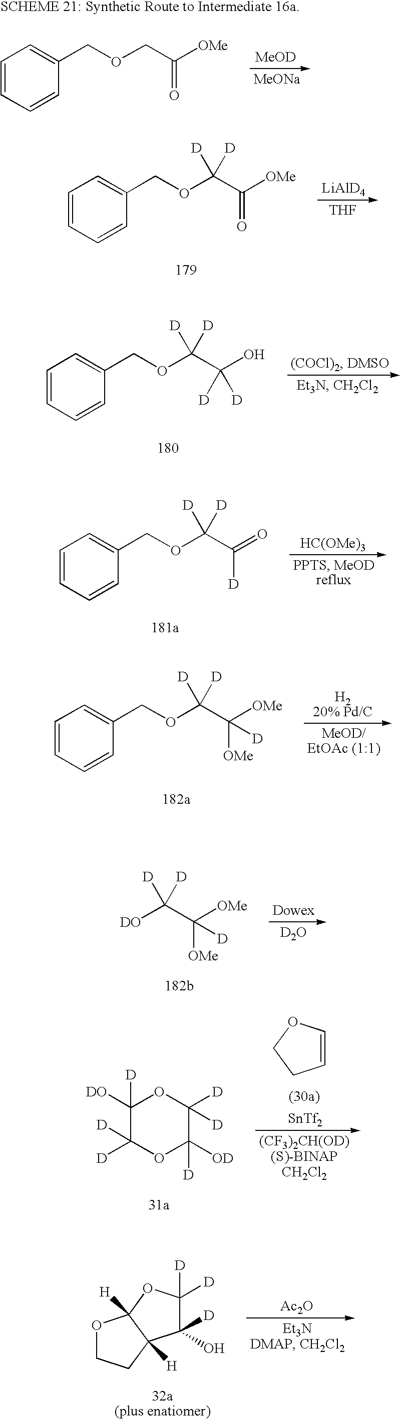

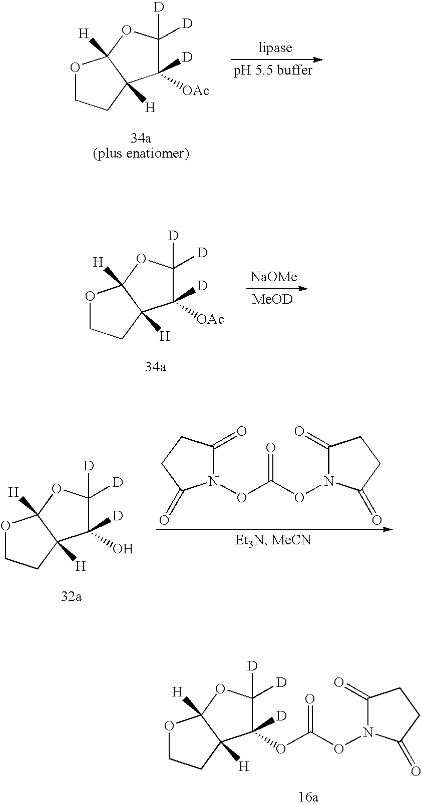

Step 1. Methyl 2-(benzyloxy)acetate. A solution of 2-(benzyloxy)acetic acid (25.0 g, 150 mmol) in MeOH (500 mL) was cooled to 4° C. in an ice-water bath. Thionyl chloride (13.0 mL, 179 mmol) was added drop-wise via an addition funnel at a rate to maintain the temperature below 8° C. The resulting solution was stirred for 30 min. at 4° C. and 2.5 hr. at room temperature. The solvent was evaporated under reduced pressure and the resulting residue was dissolved in EtOAc (200 mL) and washed with sat. aq. NaHCO₃ (200 mL). The aqueous layer was extracted with an additional portion of EtOAc (100 mL) and the organic layers were combined, washed with brine (200 mL), dried over Na₂SO₄, filtered, and evaporated under reduced pressure to afford 27.6 g (92%) of methyl 2-(benzyloxy)acetate as a clear, colorless liquid. ¹H NMR (DMSO-d6, 400 MHz): δ 7.26-7.41 (m, 5H), 4.54 (s, 2H), 4.18 (s, 2H), 3.67 (s, 3H).

Step 2. Methyl 2-(benzyloxy)-2,2-d₂-acetate (179). Sodium metal (0.32 g, 13.8 mmol) was dissolved in MeOD (150 mL; Cambridge Isotope Laboratories, 99 atom % D). To the solution was added a solution of methyl 2-(benzyloxy)acetate (25.0 g, 138 mmol) in MeOD (100 mL). The solution was heated to 40° C. and stirred for 24 hrs. The solvent was evaporated under reduced pressure and fresh MeOD (250 mL) was added. The solution was heated to 40° C. and stirred for 24 hrs. This cycle was repeated a third time. After completion of the third cycle, the solvent was evaporated under reduced pressure and the residue taken up in of EtOAc (300 mL). The solution was washed with D₂O (200 mL; Cambridge Isotope Laboratories, 99 atom % D) and brine (200 mL). The organic layer was dried over Na₂SO₄, filtered, and evaporated under reduced pressure. The crude material was purified by silica gel column chromatography on an ISCO system (20% EtOAc/hexanes). Fractions containing product were evaporated under reduced pressure to afford 22.1 g (88%) of 179 as a clear colorless liquid. ¹H NMR (DMSO-d6, 400 MHz): δ 7.25-7.40 (m, 5H), 4.54 (s, 2H), 3.67 (s, 3H).

Step 3. 2-(Benzyloxy)-1,1,2,2-d-4-ethanol (180). A solution of 179 (21.0 g, 115 mmol) in THF (450 mL) was cooled to 4° C. in an ice-water bath. To the solution, solid lithium aluminum deuteride (5.4 g, 129 mmol; Cambridge Isotope Laboratories, 98 atom % D) was added portionwise. An exotherm was observed with the temperature reaching 28° C. Upon completion of the addition, the reaction was stirred 2.5 hr in the ice-water bath. The reaction was then quenched by the slow addition of 1 N HCl (150 mL). During the quench the reaction temperature reached 20° C. The resulting suspension was filtered through a plug of Celite®, washing with MTBE. The filtrate was transferred to a separatory funnel and extracted with MTBE (2×400 mL). The combined organic layers were washed with brine (600 mL), dried over Na₂SO₄, filtered, and evaporated under reduced pressure. The crude product was purified by silica gel column chromatography on an ISCO system (2% MeOH/CH₂Cl₂). Fractions containing product were evaporated under reduced pressure to afford 18g (91%) of 180 as a clear colorless liquid. ¹H NMR (DMSO-d6, 400 MHz): δ 7.23-7.39 (m, 5H), 4.60 (s, 1H), 4.48 (s, 2H).

Step 4. 2-(Benzyloxy)-(acetaldehyde-d3) (181a). A solution of oxalyl chloride (7.50 mL, 33.2 mmol) in CH₂Cl₂ (17.2 mL) was stirred under nitrogen at −78° C. and a solution of dimethylsulfoxide (12.1 mL, 171 mmol) in CH₂Cl₂ (34.5 mL) was added over 5 min. This mixture was stirred for 20 min and a solution of 2-(benzyloxy)ethanol-d4 (180) (5.19 g, 33.2 mmol) in CH₂Cl₂ (17.2 mL) was added over 5 min. Stirring was continued for 20 min. Triethylamine (24.4 mL, 175 mmol) was slowly added and stirring was continued in the cold bath until the temperature reached −30° C. The cold bath was removed and 50% citric acid solution in D₂O was added. Stirring was continued for 5 min and the phases were separated. The organic layer was washed sequentially with 50% citric acid solution in D₂O, D₂O and 1M Na₂CO₃ in D₂O, dried over Na₂SO₄ and filtered. The solvent was removed under reduced pressure to afford 181a as a brown oil which was used without further purification. ¹H NMR (DMSO-d6, 400 MHz): δ 7.25-7.44 (m, 5H), 4.55 (s, 2H).

Step 5. ((2,2-Dimethoxy-1,1,2-d₃-ethoxy)methyl)benzene (182a). To a mixture of 181a (10.1 g, 65.8 mmol), trimethyl-orthoformate (8.64 mL, 78.9 mmol.) and MeOD (131 mL; Cambridge Isotope Laboratories, 99 atom % D), was added PPTS (913 mg, 3.63 mmol) and the mixture was refluxed under nitrogen for 3 hr. The solvent was removed under reduced pressure. The resulting residue was diluted with CD₂Cl₂, washed with 5% potassium carbonate solution and brine, dried over Na₂SO₄, filtered and the solvent was removed under reduced pressure. The residue was purified by silica gel column chromatography on an ISCO system (0 to 100% EtOAc/heptane) to afford 182a (11.0 g, 84%). ¹H NMR (DMSO-d6, 400 MHz): δ 7.24-7.43 (m, 5H), 4.51 (s, 2H), 3.28 (s, 6H).

Step 6. 2,2-Dimethoxy(ethanol-d4) (182b). A solution of 182a (5.56 g, 27.9 mmol) in 1:1 MeOD/EtOAc (250 mL) was treated with 20% palladium on activated carbon (50% wet, 4.73 g) and hydrogenated at 54 psi for 3 hrs. The reaction mixture was filtered through Celite® and the solvent was removed under reduced pressure to afford 182b (2.83 g, 93%). ¹H NMR (DMSO-d6, 400 MHz): δ 3.26 (s, 6H).

Step 7. 1,4-Dioxane-2,5-diol-d8 (31a). A solution of 182b (5.09 g, 46.7 mmol) in D₂O (72 mL) was stirred with Dowex® Marathon C (235 mg) at 50° C. for 3 hrs. The reaction mixture was filtered, the resin was discarded and the solvent was removed under reduced pressure. The crude material was purified by silica gel column chromatography on an ISCO system (0-100% MeCN/CH₂Cl₂) to afford 1.88 g (63%) of 31a as a clear oil. ¹³C NMR (DMSO-d6, 400 MHz): δ 104.1, 102.0, 94.1, 93.9, 89.7, 88.7, 70.6, 67.5, 65.5, 63.5, 62.5, 61.5.

Step 8. (3aS,6aR)-2,2,3-d₃-Hexahydrofuro[2,3-b]furan-3-ol (32a as mixture with its enantiomer). A mixture of (S)-BINAP (2.89 g, 4.67 mmol) and tin triflate (1.75 g, 4.20 mmol) in hexafluoroisopropan(ol-d) (9.76 mL) and CH₂Cl₂ (19.5 mL) was stirred under nitrogen at room temperature for 40 min. To solution of compound 31a (1.79 g, 13.9 mmol) in CH₂Cl₂ (3.57 mL) was added hexafluoroisopropan(ol-d) (1.79 mL) and stirring was continued at room temperature for 40 min. The reaction mixture was cooled in an ice-bath and 2,3-dihydrofuran (30a) (3.18 mL, 41.8 mmol) was added by syringe such that the temperature of the reaction did not rise above 11° C. Stirring was continued in the ice-bath for 20 min then at room temperature overnight. The solvents were removed under reduced pressure to give crude 32a and its enantiomer. Silica gel column chromatography on an ISCO instrument (50-80% EtOAc/heptane) afforded impure 32a and its enantiomer (3.57 g) that was used without further purification in the next step. The enantiomeric ratio was not determined at this step, but it has been reported that very similar reaction conditions applied to 31a-d0 affords 32a-d0 in 14% ee (Canoy et al. Org. Lett. 2008, 10, 1103-1106). ¹H NMR (CDCl₃, 400 MHz): δ 5.69 (d, J=4.8, 1H), 3.99 (dt, J=3.3, 8.1, 1H), 2.85 (m, 1H), 2.30 (ddd, J=3.0, 6.6, 10.1, 1H), 1.87 (m, 1H).

Step 9. (3aS,6aR)-2,2,3-d₃-Hexahydrofuro[2,3-b]furan-3-yl acetate (34a as mixture with its enantiomer). A solution of 32a and its enantiomer (3.57 g, 26.6 mmol, assuming 100% purity) in CH₂Cl₂ (89 mL) was stirred in an ice-bath under nitrogen and triethylamine (18.6 mL, 133 mmol) and 4-dimethylaminopyridine (0.315 g, 2.58 mmol) were added. To the reaction mixture was added acetic anhydride (6.29 mL, 66.5 mmol). The reaction mixture was allowed to warm to room temperature, stirred for 1 hr and then diluted with CH₂Cl₂ and washed with water, 1N HCl, water and saturated NaHCO₃. The organic layer was dried over Na₂SO₄, filtered and concentrated under reduced pressure. The crude product was purified by silica gel column chromatography on an ISCO system (0-10% EtOAc/CH₂Cl₂) to afford 3.10 g (64% over 2 steps) of 34a and its enantiomer as approximately a 1:1 mixture of epimers at the 3 position. ¹H NMR (CDCl₃, 400 MHz): δ 5.73 (d, J=5.3, 1H), 3.99 (dt, J=2.5, 8.6, 1H), 3.91 (m, 1H), 3.06 (m, 1H), 2.09 (s, 3H), 1.97-2.03 (m, 1H), 1.90 (m, 1H).

Step 10. (3aS,6aR)-2,2,3-d₃-Hexahydrofuro[2,3-b]furan-3-yl acetate (34a). Compound 34a and its enantiomer (0.271 g, 1.55 mmol) in pH 5.5 buffer (1.2 mL) [prepared from a solution of sodium dihydrogen phosphate (5.37 g) in water (30g) that was adjusted to pH 5.5 with 30% NaOH] was treated with Novozyme 435 (63 mg) and stirred at 40-43° C. for 1 hr. The polymer was filtered off and washed with 15% isopropanol in water. The filtrate was diluted with water and extracted with CH₂Cl₂ (2×25 mL). The combined organic solutions were washed with H₂O (2×25 mL) and brine (1×25 mL), dried (Na₂SO₄), filtered and concentrated under reduced pressure. The crude product was purified by silica gel column chromatography on an ISCO system (0-10% EtOAc/CH₂Cl₂) to afford 96 mg (35%) of 34a. ¹H NMR (CDCl₃, 400 MHz): δ 5.72 (d, J=4.8, 1H), 3.98 (dt, J=2.3, 8.1, 1H), 3.90 (m, 1H), 3.05 (m, 1H), 2.10 (s, 3H), 1.96-2.05 (m, 1H), 1.83-1.96 (m, 1H).

Step 11. (3aS,6aR)-2,2,3-d₃-Hexahydrofuro[2,3-b]furan-3-ol (32a). A solution of 34a (96.0 mg, 0.548 mmol) in MeOD (1.0 mL) was stirred under nitrogen at room temperature and NaOMe (5 mg, 0.0926 mmol) was added. The reaction was stirred for 1 h and then AcOH (6 μL) was added. The solvent was removed under reduced pressure and the crude product was purified by silical gel column chromatography on an ISCO system (50-80% EtOAc/heptane) to afford 72 mg (98%) of 32a (>99% ee by chiral GC [Column: Chiraldex G-TA, Temperature: 140° C. (isothermal), Flow rate: 1 mL/min]. ¹H NMR (CDCl₃, 400 MHz): δ 5.68 (d, J=5.2, 1H), 3.98 (dt, J=2.8, 8.6, 1H), 3.89 (dt, J=6.4, 9.6, 1H), 2.85 (m, 1H), 2.30 (tdd, J=2.5, 6.3, 12.9, 1H), 1.87 (m, 1H).

Step 12. 2,5-Dioxopyrrolidin-1-yl-(3R,3aS,6aR)-2,2,3-d₃-Hexahydrofuro[2,3-b]furan-3-yl-carbonate (16a). A solution of 32a (64.0 mg, 0.477 mmol) in acetonitrile (0.954 mL) was stirred under nitrogen at room temperature and N,N′-disuccinylimidyl carbonate (183 mg, 0.716 mmol) was added. The resulting suspension was treated with triethylamine (0.133 mL, 0.954 mmol) and the mixture stirred at room temperature overnight. The solvent was removed under reduced pressure and the residue partitioned between sat. NaHCO₃ and EtOAc. Phases were separated and the aqueous phase was extracted with EtOAc. The combined organic extracts were washed with brine, dried over Na₂SO₄, filtered and the solvent was removed under reduced pressure. The crude product was purified by silica gel column chromatography on an ISCO system (0-10% EtOAc/CH₂Cl₂) to afford 0.104g (79%) of 16a. ¹H NMR (CDCl₃, 400 MHz): δ 5.75 (d, J=5.1, 1H), 4.04 (dt, J=2.0, 8.3, 1H), 3.90-3.99 (m, 1H), 3.13 (m, 1H), 2.86 (s, 4H), 2.15 (m, 1H), 1.92-2.03 (m, 1H). MS (ESI) 275.1 [(M+H)⁺], 297.0 [(M+Na)⁺].

### Example 4B

Synthesis of 2,5-Dioxopyrrolidin-1-yl-(3R,3aS,6aR)-2,2,3,4,4,5,5-d7-hexahydrofuro[2,3-b]furan-3-yl-carbonate (16b). Intermediate 16b was prepared as shown in Scheme 22 and as described below.

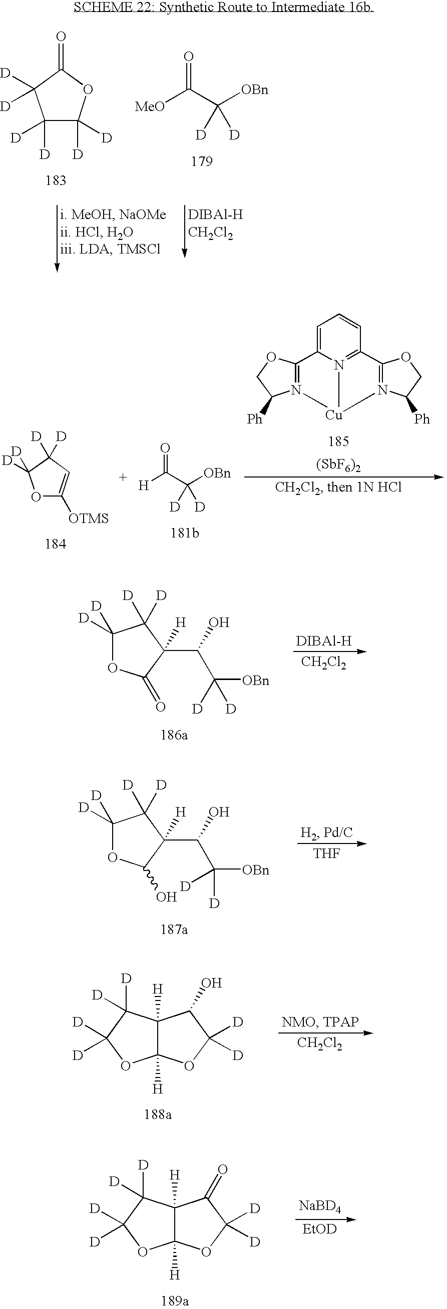

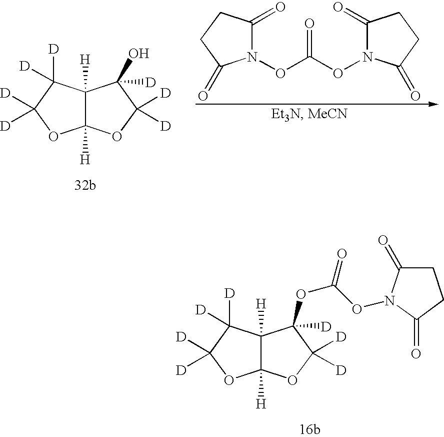

Step 1. 2-(Benzyloxy)-2,2-d₂-acetaldehyde (181b). 179 (1.0 g, 5.5 mmol) was dissolved in CH₂Cl₂ (10 mL). The mixture was cooled to −78° C. and DIBAl-H (6.0 mL, 1M in CH₂Cl₂, 6.0 mmol) was added. After 2 hours the reaction (monitored by TLC) was not complete. Additional DIBAl-H (1.0 mL, 1M in CH₂Cl₂) was added and the mixture was stirred at −78° C. for 1 hour. The reaction was quenched with water (10 mL), diluted with CH₂Cl₂ (50 mL) then saturated aqueous potassium sodium tartrate (100 mL) was added. The resulting mixture was allowed to warm to room temperature. The organic phase was separated and the aqueous phase was extracted with CH₂Cl₂ (3×50 mL). The combined organic layers were dried over Na₂SO₄, filtered, and concentrated under reduced pressure to afford 181b as a clear oil (700 mg, 83%) which was used without purification.

Step 2. Trimethyl-(4,4,5,5-d₄-4,5-dihydrofuran-2-yloxy)silane (184). Sodium metal (2.10 g, 90.6 mmol) was dissolved in MeOH (180 mL). To this was added γ-butyrolactone-d₆ (15.0 g, 163 mmol; Aldrich, 98 atom % D) as a solution in MeOH (180 mL). The resulting solution was heated at reflux for 16 hours. The reaction was cooled to room temperature and concentrated under reduced pressure. A fresh portion of MeOH (360 mL) was added to the residue and the reaction was heated at reflux for an additional 16 hours. The reaction was cooled to room temperature and quenched by the addition of acetic acid (5.30 mL, 90.0 mmol) and several drops of concentrated HCl. The solvent was evaporated under reduced pressure and CH₂Cl₂ (100 mL) was added. The resulting suspension was filtered and the filtrate was evaporated under reduced pressure leaving a slightly yellow oil (γ-hydroxy-methylbutyrate-d₄). This material was dissolved in H₂O (100 mL) and concentrated HCl (9 mL) was added. The reaction was heated at reflux for 1 hour and then cooled to room temperature. The solution was saturated with NaCl and the product extracted with CH₂Cl₂ (3×500 mL). The combined organic layers were dried over Na₂SO₄, filtered, and evaporated under reduced pressure to afford 15.1 g of a yellow oil. The crude product was purified by Kugelrohr distillation to afford γ-butyrolactone-d₄ (13.9 g, 94%) as a colorless liquid.

A solution of diisopropylamine (9.9 g, 98 mmol) in THF (89 mL) was cooled to 0° C. and a solution of n-butyllithium (36 mL, 2.5M in hexane, 90 mmol) was added dropwise over 10 minutes. The resulting yellow solution was stirred at 0° C. for 15 minutes and then cooled to −78° C. To this was added a solution of γ-butyrolactone-d₄ (7.5 g, 83 mmol) and trimethylsilyl chloride (10.6 g, 98 mmol) in THF (56 mL) over 5 minutes. The reaction was warmed to room temperature and stirred for 2.5 hours. The majority of the THF was evaporated under reduced pressure and hexane was added to the remaining suspension. The solids were removed by filtration through a pad of Celite®. The filtrate was concentrated under reduced pressure to provide a yellow oil. This material was purified by Kugelrohr distillation (bp 80° C. at 15 torr) affording 4.5 g (33%) of 184 as a colorless oil. MS (ESI) 162 [(M+H)⁺].

Step 3. (S)-3-((S)-2-(Benzyloxy)-2,2-d₂-1-hydroxyethyl)-4,4,5,5-d₄-dihydrofuran-2(3H)-one (186a). A solution of [Cu((S,S)-Phenyl-bis(oxazolinyl)-pyridine)](SbF₆)₂ (0.05 equiv.; prepared according to JACS, 1996, 118, 5814) was cooled to −78° C. and 181b (1 equiv.) and 184 (1.2 equiv.) were then added. The reaction was stirred 1 hour and then warmed to room temperature. The crude reaction mixture was filtered through a plug of silica gel washing with MTBE and the filtrate was concentrated under reduced pressure. The resulting oil was dissolved in THF (10 mL) and 1 N HCl (3 mL) was added. The mixture was stirred at room temperature for 15 minutes and then diluted with H₂O (15 mL). The aqueous mixture was extracted with MTBE (2×30 mL). The combined organic layers were washed with saturated aqueous NaHCO₃ (1×30 mL) and brine (1×30 mL), dried over Na₂SO₄, filtered, and concentrated under reduced pressure to afford a slightly yellow oil. The crude material was purified by silica gel column chromatography on an Analogix system (30-60% EtOAc/heptane). Fractions containing product were evaporated under reduced pressure affording 186a (63%) as a colorless oil.

Step 4. (35)-3-((S)-2-(Benzyloxy)-2,2-d₂-1-hydroxyethyl)-4,4,5,5-d₄-tetrahydrofuran-2-ol (187a). Compound 186a (1 equiv.) was dissolved in CH₂Cl₂ (0.12 M) and the solution cooled to −78° C. A solution of diisobutylaluminum hydride (1M in CH₂Cl₂, 2.5 equiv.) was added over a period of 5 minutes. The reaction was stirred for 45 minutes then quenched by the slow addition of MeOH (2 mL) and saturated aqueous potassium sodium tartrate (0.5 mL). The mixture was warmed to room temperature and stirred for 30 minutes. The turbid solution was filtered through Celite® washing with CH₂Cl₂. The filtrate was concentrated under reduced pressure affording 187a (100%) as a white solid which was used crude in the next step.

Step 5. (3S,3aS,6aR)-2,2,4,4,5,5-d₆-hexahydrofuro[2,3-b]furan-3-ol (188a). To a solution of 187a (1 equiv.) in THF (0.04 M) was added 20% Pd/C, (50% wet, 20 wt %). The resulting mixture was hydrogenated at 25 psi of H₂ for 30 minutes. After TLC analysis confirmed complete consumption of starting material, the reaction mixture was filtered through a pad of Celite® under a stream of N₂ washing with THF. The filtrate was evaporated affording 188a (61%) as a slightly yellow oil which was used without further purification in the next step.

Step 6. (3aR,6aR)-2,2,4,4,5,5-d₆-tetrahydrofuro[2,3-b]furan-3(2H)-one (189a). To a cold (0° C.) solution of 188a (1 equiv.) in CH₂Cl₂ (0.04M) was added 4 A molecular sieves, tetrapropylammonium perruthenate (0.1 equiv.), and 4-methylmorpholine-N-oxide (1.5 equiv.). The brown/black reaction was warmed to room temperature and stirred for 2 hours. The crude reaction mixture was then passed through a short plug of silica gel eluting with 50% EtOAc/heptane. The filtrate was evaporated under reduced pressure yielding 189a (58%) as a colorless oil which solidified upon standing.

Step 7. (3R,3aS,6aR)-2,2,3,4,4,5,5-d₇-hexahydrofuro[2,3-b]furan-3-ol (32b). A solution of 189a (1 equiv.) in EtOD (0.04M; Aldrich, 99.5 atom % D) was cooled to −15° C. Sodium borodeuteride (1.4 equiv.; Cambridge Isotope Laboratories, 99 atom % D) was added in one portion and stirring was continued for 2.5 hours while allowing the solution to gradually warm to room temperature. The reaction was then quenched by the addition of saturated aqueous sodium chloride. The mixture was extracted with EtOAc (3×50 mL) and the combined organic layers were dried over Na₂SO₄, filtered, and concentrated under reduced pressure to afford 32b as a light yellow oil. MS (ESI) 136 [(M+H)⁺], 158 [(M+Na)⁺].

Step 8. 2,5-Dioxopyrrolidin-1-yl-(3R,3aS,6aR)-2,2,3,4,4,5,5-d₇-hexahydrofuro[2,3-b]furan-3-yl-carbonate (16b). Prepared from alcohol 32b according to the final step in the synthesis of 16a above. MS (ESI) 279.0 [(M+H)⁺], 301.2 [(M+Na)⁺].

### Example 4C

Synthesis of 2,5-Dioxopyrrolidin-1-yl-(3R,3aS,6aR)-(hexahydrofuro[2,3-b]furan-d₉)-3-yl-carbonate (16c). Intermediate 16c was prepared as shown in Scheme 23 and as described below.

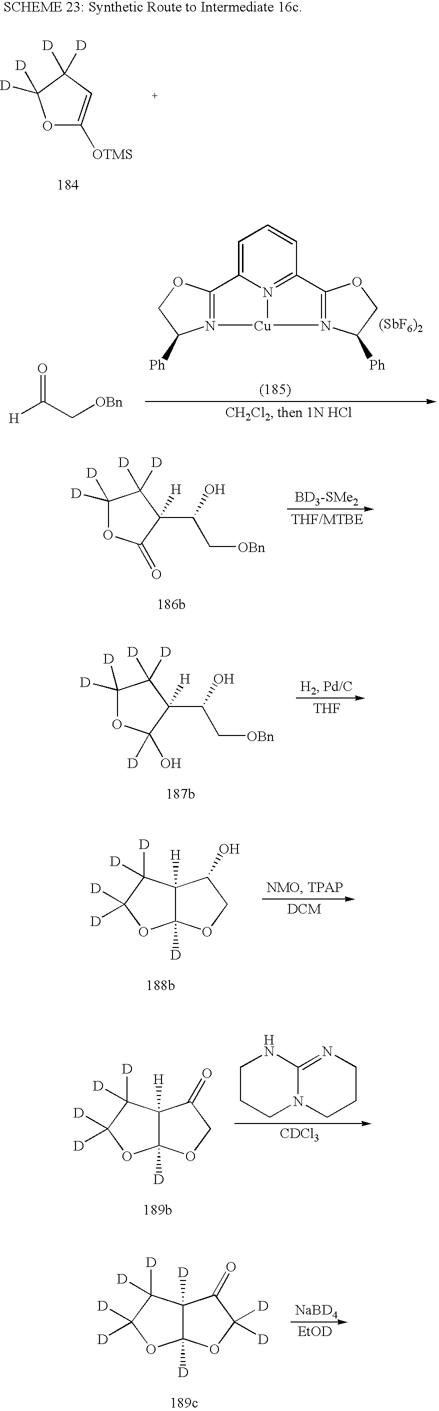

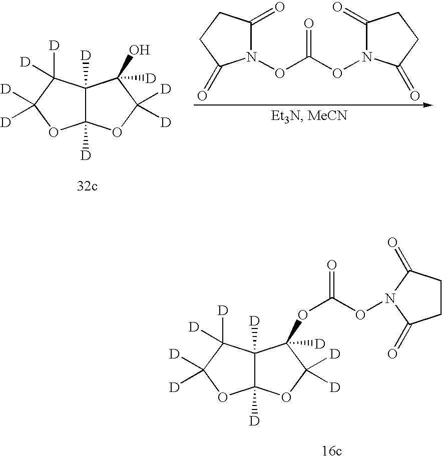

Step 1. (S)-3-((S)-2-(Benzyloxy)-1-hydroxyethyl)-4,4,5,5-d₄-dihydro furan-2(3H)-one (186b). A 0.0125 M solution of [Cu((S,S)-Phenyl-bis(oxazolinyl)pyridine)](SbF₆)₂, 185 (125 mL, 1.6 mmol) [prepared according to the method described in JACS, 1996, 118, 5814] was cooled to −78° C. Benzyloxyacetaldehyde (3.0 g, 20 mmol, purified by Kugelrohr distillation prior to use), and 184 (3.8 g, 23 mmol) were added. The reaction was stirred for 3 hours then warmed to room temperature and filtered through a plug of silica gel washing with MTBE. The filtrate was concentrated under reduced pressure and the resulting oil was dissolved in THF (200 mL) and 1 N HCl (25 mL) was added. The mixture was stirred at room temperature for 15 min and then diluted with H₂O (250 mL). The aqueous mixture was extracted with MTBE (2×500 mL) and the combined organic layers were washed with saturated aqueous NaHCO₃ (1×300 mL) and brine (1×300 mL), dried over Na₂SO₄, filtered, and evaporated under reduced pressure to give a slightly yellow oil. The crude product was purified by silica gel column chromatogrpahy on an Analogix system (30-80% EtOAc/heptane). Fractions containing product were evaporated under reduced pressure to give 3.1 g (65%) of 186b as a colorless oil. MS (ESI) 241 [(M+H)⁺].

Step 2. (35)-3-45)-2-(Benzyloxy)-1-hydroxyethyl)-2,4,4,5,5-d₅-tetrahydrofuran-2-ol (187b). A solution of 186b (1.8 g, 7.5 mmol) in THF/MTBE (15 mL/15 mL) was cooled to 0° C. and BD₃-SMe₂ (0.74 mL, 7.5 mmol; Cambridge Isotope Laboratories, 99 atom % D) was added. The reaction mixture was allowed to warm to room temperature and stirred overnight. The reaction mixture was quenched with MeOD (5 mL; Cambridge Isotope Laboratories, 99 atom % D) and the solvent was evaporated under reduced pressure. The crude product was purified by silica gel column chromatography on an Analogix system (20-80% EtOAc/heptane). Unreacted starting material (1.0 g) eluted first followed by the desired product (400 mg). The unreacted starting material was re-subjected to these reaction conditions a total of 3 times to give a total of 930 mg (51%) 187b as colorless oil. MS (ESI) 266 [(M+Na)⁺].

Step 3. (3S,3aS,6aR)-4,4,5,5,6a-d5-hexahydrofuro[2,3-b]furan-3-ol (188b). A 500 ml, Parr shaker bottle was charged with 20% Pd/C (50% wet, 180 mg). The catalyst was washed with D₂O (2×20 mL; Cambridge Isotope Laboratories, 99.9 atom % D) and THF (2×20 mL). To the catalyst was added a solution of 32 (930 mg, 3.80 mmol) in THF (50 mL). The resulting mixture was hydrogenated at 25 psi H₂ for 2 hr. After TLC analysis confirmed complete consumption of starting material, the reaction mixture was filtered through a pad of Celite® under a stream of N₂ washing the pad with THF. The filtrate was evaporated under reduced pressure to give 530 mg (quantitative) of 33 as a white solid which was used without further purification in the next step. MS (ESI) 136 [(M+H)⁺].

Step 4. (3aR,6aR)-4,4,5,5,6a-d₅-Tetrahydrofuro[2,3-b]furan-3(2H)-one (189b). To a cold (0° C.) solution of 33 (470 mg, 3.5 mmol) in CH₂Cl₂ (80 mL) was added 4 A molecular sieves (3g), tetrapropylammonium perruthenate (124 mg, 0.350 mmol), and 4-methylmorpholine-N-oxide (614 mg, 5.30 mmol). The brown/black reaction mixture was warmed to room temperature and stirred for 2 hr. The crude reaction mixture was then passed through a short plug of silica gel eluting with 50% EtOAc/heptane. The filtrate was evaporated under reduced pressure to give 450 mg (97%) of 34 as a white solid.

Step 5. (3aR,6aR)-Tetrahydrofuro[2,3-b]furan-3(2H)-one-d₈ (189c). To a solution of 189b (450 mg. 3.40 mmol) in CDCl₃ (40 mL; Cambridge Isotope Laboratories, 99.8 atom % D) was added 2,3,4,6,7,8-hexahydro-1H-pyrimido[1,2-a]pyrimidine (41 mg, 0.34 mmol). The reaction was stirred for 3 hr at room temperature during which time a yellow color developed. Silica gel was added to the reaction mixture and the solvent was evaporated under reduced pressure. The crude material was purified by silica gel column chromatography on an Analogix system (20-50% EtOAc/heptane). Fractions containing product were evaporated to provide 290 mg (63%) of 189c as a white solid. MS (ESI) 136 [(M+H)⁺].

Step 6. (3R,3aS,6aR)-Hexahydrofuro[2,3-b]furan-3-ol-d₉ (32c). A solution of 189c (250 mg, 1.80 mmol) in EtOD (5 mL; Aldrich, 99 atom atom % D) was cooled to −15° C. Sodium borodeuteride (85 mg, 2.02 mmol; Cambridge Isotope Laboratories, 99 atom % D) was added portionwise and stirring was continued for 2.5 hr while the solution gradually warmed to room temperature. The reaction was then quenched by the addition of acetone (10 mL) and MeOH (10 mL). The solvent was evaporated under reduced pressure and the crude material redissolved in MeOH and evaporated. This process was repeated 8 times. The crude product was adsorbed onto silica gel and purified by silica gel column chromatography on an Analogix system (0-5% MeOH/CH₂Cl₂). Product fractions were evaporated under reduced pressure to provide 210 mg (84%) 32c as colorless oil. MS (ESI) 140 [(M+H)⁺].

Step 7. 2,5-Dioxopyrrolidin-1-yl-(3R,3aS,6aR)-(hexahydrofuro[2,3-b]furan-3-yl-d₉)-carbonate (16c). To a solution of 32c (230 mg, 1.65 mmol) in MeCN (8 mL) was added N,N′-disuccinimidyl carbonate (640 mg, 2.48 mmol) and triethylamine (0.5 mL, 3.3 mmol). The resulting slightly brown solution was stirred at room temperature overnight. The solvent was evaporated under reduced pressure and the residue dissolved in EtOAc. This solution was washed with sat. aq. NaHCO₃, brine, dried over Na₂SO₄, filtered, and evaporated. The crude product was adsorbed onto silica gel and purified by silica gel column chromatography on an Analogix system (0-30% EtOAc/CH₂Cl₂). Product fractions were evaporated under reduced pressure yielding 180 mg of a colorless film. This material was dissolved in a minimal amount of CH₂Cl₂ and diluted with hexane. Slow evaporation of this solution gave 160 mg (35%) of 16c as a white solid. MS (ESI) 303 [(M+Na)⁺].

### Example 5

Synthesis of (3R,3aS,6aR)-Hexahydrofuro[2,3-b]furan-3-yl-((2S,3R)-3-hydroxy-4-(N-isobutyl-phenylsulfonamido)-1-phenylbutan-2-yl)carbamate-d12 (Compound 220) and general route for Examples 6-39.

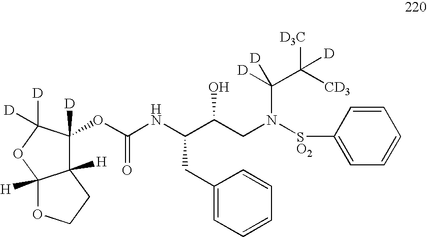

Compound 220 and Examples 6-39 were prepared as outlined in Scheme 15 above and as described below. Intermediates for use in the syntheses are described in the table below.

| Intermediate # | R² | R³ | R⁴ |
|---|---|---|---|
| 12a | H | iPr—D₇ | — |
| 12b | H | Cyclopentyl-D₉ | — |
| 12c | p-OCD₃ | iPr—D₇ | — |
| 12d | p-OD₇iPr | iPr—D₇ | — |
| 12e | p-OD₂Bn | iPr—D₇ | — |
| 14a | H | iPr—D₇ | Ph |
| 14b | H | iPr—D₇ | 3,4-diF—Ph |
| 14c | H | iPr—D₇ | 3-F-4-OCD₃—Ph |
| 14d | H | iPr—D₇ | 4-OCD₃—Ph |
| 14e | H | iPr—D₇ | 4-OCF₃—Ph |
| 14f | H | iPr—D₇ | 4-OCHF₂—Ph |
| 14g | H | iPr—D₇ | 4-CN—Ph |
| 14h | H | iPr—D₇ | 4-CF₃ |
| 14i | H | iPr—D₇ | 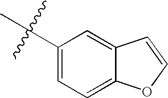 |
| 14j | H | iPr—D₇ | 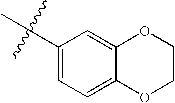 |
| 14k | H | iPr—D₇ | 4-NHAc—Ph |
| 14-l | H | iPr—D₇ | 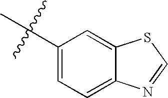 |
| 14m | H | iPr—D₇ | 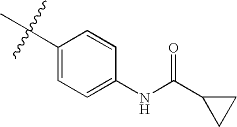 |
| 14n | H | iPr—D₇ | 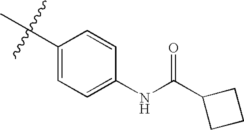 |
| 14o | H | iPr—D₇ | 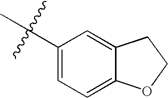 |
| 14p | H | iPr—D₇ | 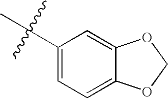 |
| 14q | H | Cyclopentyl-D₉ | 4-NHAc—Ph |
| 14r | p-OCD₃ | iPr—D₇ | 3,4-diF—Ph |
| 14s | p-OCD₃ | iPr—D₇ | 3-F-4-OCD₃—Ph |
| 14t | p-OCD₃ | iPr—D₇ | 4-OCD₃—Ph |
| 14u | p-OCD₃ | iPr—D₇ | 4-OCF₃—Ph |
| 14v | p-OCD₃ | iPr—D₇ | 4-OCHF₂—Ph |
| 14w | p-OiPr—D₇ | iPr—D₇ | 3,4-diF—Ph |
| 14x | p-OiPr—D₇ | iPr—D₇ | 3-F-4-OCD₃—Ph |
| 14y | p-OiPr—D₇ | iPr—D₇ | 4-OCD₃—Ph |
| 14z | p-OiPr—D₇ | iPr—D₇ | 4-OCF₃—Ph |
| 14aa | p-OiPr—D₇ | iPr—D₇ | 4-OCHF₂—Ph |
| 14bb | p-OBn—D₂ | iPr—D₇ | 3,4-diF—Ph |
| 14cc | p-OBn—D₂ | iPr—D₇ | 3-F-4-OCD₃—Ph |
| 14dd | p-OBn—D₂ | iPr—D₇ | 4-OCD₃—Ph |
| 14ee | p-OBn—D₂ | iPr—D₇ | 4-OCF₃—Ph |
| 14ff | p-OBn—D₂ | iPr—D₇ | 4-OCHF₂—Ph |

Step 1. tert-Butyl-((2S,3R)-3-hydroxy-4-(isobutylamino)-1-phenylbutan-2-yl)carbamate-d9 (12a). Epoxide 10a (1.83 g, 6.95 mmol) was added to a pressure flask containing a solution of amine 11a (1.14 g, 13.9 mmol) in isopropanol (33.0 mL). The reaction was then sealed and heated to 65° C. for 15 hours. After cooling to room temperature, the reaction was concentrated to dryness and purified by silica gel column chromatography on an ISCO system (0-10% MeOH/CH₂Cl₂) to afford amino alcohol 12a (1.80 g, 75%) as a white solid. MS (ESI) 346.4 [(M+H)⁺].

Step 2. tert-Butyl-((2S,3R)-3-hydroxy-4-(N-isobutylphenylsulfonamido)-1-phenylbutan-2-yl)carbamate-d9 (14a). To a solution of amino alcohol 12a (118 mg, 0.342 mmol) in CH₂Cl₂ (6.00 mL) was added phenyl sulfonyl chloride 13a (66 μL, 0.513 mmol) followed by N,N-diisopropylethylamine (119 μL, 0.684 mmol). The reaction stirred at room temperature for 15 hours then was concentrated under vacuum. The resulting residue was purified by silica gel column chromatography on an ISCO system (0-40% EtOAc/heptane) to afford sulfonamide 14a (160 mg, 96%) as a white solid. MS (ESI) 386.2 [(M-Boc+H)⁺].

Step 3. (3R,3aS,6aR)-Hexahydrofuro[2,3-b]furan-3-yl-((2S,3R)-3-hydroxy-4-(N-isobutyl-phenylsulfonamido)-1-phenylbutan-2-yl)carbamate-d12 (Compound 220). A solution of 4 M HCl in dioxane (5.00 mL) was added to sulfonamide 14a (160 mg, 0.329 mmol) and the reaction stirred at room temperature for 2 hours then was concentrated under vacuum to afford a white solid. Dicholoromethane was added to the resulting solid followed by 16a (108 mg, 0.395 mmol) and triethylamine (0.138 mL, 0.987 mmol). The reaction stirred at room temperature for 15 hours then was diluted with water 25 mL and extracted with CH₂Cl₂ (3×25 mL). The organic layers were combined, dried (Na₂SO₄), filtered through Celite® and concentrated. The resulting residue was purified by silica gel column chromatography on an ISCO system (0-80% EtOAc/heptane) to afford Compound 220 (100 mg, 56%) as a white solid. MS (ESI) 545.3 [(M+H)⁺]. ¹H NMR (CDCl₃, 400 MHz, rotamers) δ 7.83-7.73 (m, 2H), 7.61 (t, J=7.6 Hz, 1H), 7.53 (t, J=7.6 Hz, 2H), 7.32-7.25 (m, 2H), 7.25-7.17 (m, 3H), 5.77-5.60 (m, 1H), 5.05-4.93 (m, 1H), 4.07-3.74 (m, 3H), 3.73-3.61 (m, 2H), 3.25-2.94 (m, 3H), 2.93-2.71 (m, 2H), 2.05-1.74 (m, 1H), 1.70-1.54 (m, 1H), 1.52-1.40 (m, 1H).

### Example 6

Synthesis of (3R,3aS,6aR)-hexahydrofuro[2,3-b]furan-3-yl-((2S,3R)-4-(3,4-difluoro-N-isobutyl-phenylsulfonamido)-3-hydroxy-1-phenylbutan-2-yl)carbamate-d12 (Compound 211).

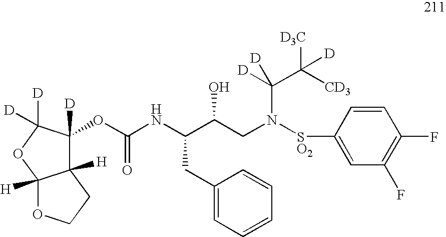

Step 1. tert-Butyl-((2S,3R)-4-(3,4-difluoro-N-isobutylphenylsulfonamido)-3-hydroxy-1-phenylbutan-2-yl)carbamate-d9 (14b). Followed step 2 for Example 5 employing amino alcohol 12a and sulfonyl chloride 13b to afford sulfonamide 14b (84%) as a white solid. MS (ESI) 422.2 [(M-Boc+H)⁺].

Step 2. (3R,3aS,6aR)-hexahydrofuro[2,3-b]furan-3-yl-((2S,3R)-4-(3,4-difluoro-N-isobutyl-phenylsulfonamido)-3-hydroxy-1-phenylbutan-2-yl)carbamate-d12 (211). Followed step 3 for Example 5 employing sulfonamide 14b and carbonate 16a to afford 211 (52%) as a white solid. MS (ESI) 581.3 [(M+H)⁺]. ¹H NMR (CDCl₃, 400 MHz, rotamers) δ 7.67-7.50 (m, 2H), 7.39-7.15 (m, 6H), 5.72-5.61 (m, 1H), 4.95 (br d, J=8.3 Hz, 1H), 3.99 (dt, J=2.5, 8.3 Hz, 0.2H), 3.95-3.79 (m, 2.8H), 3.73-3.61 (m, 1H), 3.51 (d, J=2.8 Hz, 1H), 3.20-2.97 (m, 3H), 2.93-2.71 (m, 2H), 2.35-2.25 (m, 0.4H), 1.94-1.81 (m, 0.6H), 1.71-1.55 (m, 1H), 1.49-1.37 (m, 1H).

### Example 7

Synthesis of (3R,3aS,6aR)-hexahydrofuro[2,3-b]furan-3-yl-((2S,3R)-4-(3-fluoro-N-isobutyl-4-methoxyphenylsulfonamido)-3-hydroxy-1-phenylbutan-2-yl)carbamate-d15 (Compound 207).

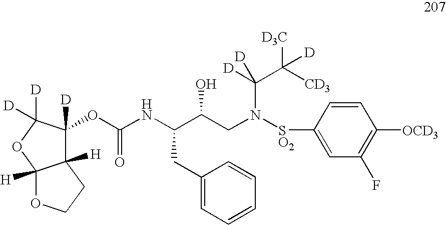

Step 1. tert-Butyl-((2S,3R)-4-(3-fluoro-N-isobutyl-4-methoxyphenylsulfonamido)-3-hydroxy-1-phenylbutan-2-yl)carbamate-d12 (14c). Followed step 2 for Example 5 employing amino alcohol 12a and sulfonyl chloride 134 to afford sulfonamide 14c (76%) as a white solid. MS (ESI) 437.3 [(M-Boc+H)⁺].

Step 2. (3R,3aS,6aR)-hexahydrofuro[2,3-b]furan-3-yl-((2S,3R)-4-(3-fluoro-N-isobutyl-4-methoxyphenylsulfonamido)-3-hydroxy-1-phenylbutan-2-yl)carbamate-d15 (207). Followed step 3 for Example 5 employing sulfonamide 14c and carbonate 16a to afford 207 (72%) as a white solid. MS (ESI) 596.3 [(M+H)⁺]. ¹H NMR (CDCl₃, 400 MHz, rotamers) δ 7.59-7.51 (m, 1H), 7.48 (dd, J=2.0, 10.4 Hz, 1H), 7.35-7.16 (m, 5H), 7.05 (t, J=8.3 Hz, 1H) 5.72-5.61 (m, 1H), 4.94 (br d, J=8.6 Hz, 1H), 3.95-3.79 (m, 3H), 3.73-3.61 (m, 1H), 3.59 (d, J=2.5 Hz, 1H), 3.19-2.94 (m, 3H), 2.93-2.85 (m, 1H), 2.85-2.74 (m, 1H), 1.71-1.55 (m, 1H), 1.49-1.40 (m, 1H).

### Example 8

Synthesis (3R,3aS,6aR)-hexahydrofuro[2,3-b]furan-3-yl-((2S,3R)-4-(N-isobutyl-4-methoxy-phenylsulfonamido)-3-hydroxy-1-phenylbutan-2-yl)carbamate-d15 (Compound 203).

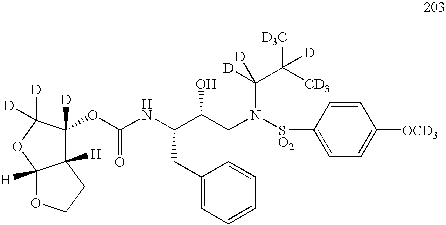

Step 1. tert-Butyl-((2S,3R)-4-(N-isobutyl-4-methoxyphenylsulfonamido)-3-hydroxy-1-phenylbutan-2-yl)carbamate-d12 (14d). Followed step 2 for Example 5 employing amino alcohol 12a and sulfonyl chloride 131 to afford sulfonamide 14d (87%) as a white solid. MS (ESI) 419.4 [(M-Boc+H)⁺].

Step 2. (3R,3aS,6aR)-hexahydrofuro[2,3-b]furan-3-yl-((2S,3R)-4-(N-isobutyl-4-methoxy-phenylsulfonamido)-3-hydroxy-1-phenylbutan-2-yl)carbamate-d15 (203). Followed step 3 for Example 5 employing sulfonamide 14d and carbonate 16a to afford 203 (74%) as a white solid. MS (ESI) 578.3 [(M+H)⁺]. ¹H NMR (CDCl₃, 400 MHz, rotamers) δ 7.71 (d, J=9.1 Hz, 2H), 7.34-7.16 (m, 5H), 6.98 (d, J=8.8 Hz, 2H), 5.70 (d, J=5.3 Hz, 0.3H), 5.64 (d, J=5.1H, 0.7H), 4.95 (br d, J=8.8 Hz, 1H), 3.99 (dt, J=2.5, 8.3 Hz, 0.4H), 3.95-3.79 (m, 2.6H), 3.73-3.61 (m, 2H), 3.20-2.97 (m, 2H), 2.93-2.73 (m, 3H), 2.35-2.25 (m, 0.4H), 1.94-1.81 (m, 0.6H), 1.71-1.55 (m, 1H), 1.49-1.37 (m, 1H).

### Example 9

Synthesis (3R,3aS,6aR)-hexahydrofuro[2,3-b]furan-3-yl-((2S,3R)-3-hydroxy-4-(N-isobutyl-4-(trifluoromethoxy)phenylsulfonamido)-1-phenylbutan-2-yl)carbamate-d12 (Compound 219).

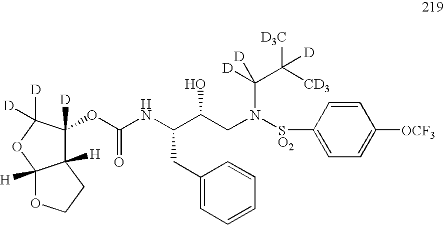

Step 1. tert-butyl-((2S,3R)-3-hydroxy-4-(N-isobutyl-4-(trifluoromethoxy)phenylsulfon-amido)-1-phenylbutan-2-yl)carbamate-d9 (5e). Followed step 2 for Example 5 employing amino alcohol 12a and sulfonyl chloride 13c to afford sulfonamide 14e (76%) as a white solid. MS (ESI) 470.4 [(M-Boc+H)⁺].

Step 2. (3R,3aS,6aR)-hexahydrofuro[2,3-b]furan-3-yl-((2S,3R)-3-hydroxy-4-(N-isobutyl-4-(trifluoromethoxy)phenylsulfonamido)-1-phenylbutan-2-yl)carbamate-d12 (219). Followed step 3 for Example 5 employing sulfonamide 14e and carbonate 16a to afford 219 (47%) as a white solid. MS (ESI) 629.3 [(M+H)⁺]. ¹H NMR (CDCl₃, 400 MHz, rotamers) δ 7.83 (d, J=8.8 Hz, 2H), 7.34 (d, J=8.3 Hz, 2H), 7.31-7.17 (m, 5H), 5.75-5.59 (m, 1H), 5.10-4.93 (m, 1H), 4.00-3.53 (m, 5H), 3.23-3.93 (m, 3H), 2.93-2.70 (m, 2H), 1.71-1.55 (m, 1H), 1.49-1.36 (m, 1H).

### Example 10

Synthesis (3R,3aS,6aR)-hexahydrofuro[2,3-b]furan-3-yl-((2S,3R)-4-(N-isobutyl-4-(difluoro-methoxy)phenylsulfonamido)-3-hydroxy-1-phenylbutan-2-yl)carbamate-d12 (Compound 217).

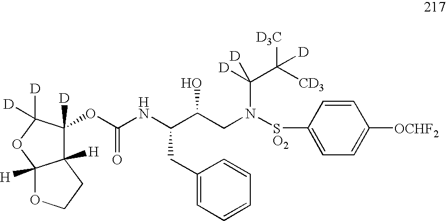

Step 1. tert-butyl-((2S,3R)-4-(N-isobutyl-4-(difluoromethoxy)phenylsulfonamido)-3-hydroxy-1-phenylbutan-2-yl)carbamate-d9 (14f). Followed step 2 for Example 5 employing amino alcohol 12a and sulfonyl chloride 13d to afford sulfonamide 14f (78%) as a white solid. MS (ESI) 452.2 [(M-Boc+H)⁺].

Step 2. (3R,3aS,6aR)-hexahydrofuro[2,3-b]furan-3-yl-((2S,3R)-4-(N-isobutyl-4-(difluoro-methoxy)phenylsulfonamido)-3-hydroxy-1-phenylbutan-2-yl)carbamate-d12 (217). Followed step 3 for Example 5 employing sulfonamide 14f and carbonate 16a to afford 217 (52%) as a white solid. MS (ESI) 611.3 [(M+H)⁺]. ¹H NMR (CDCl₃, 400 MHz, rotamers) δ 7.79 (d, J=8.8 Hz, 2H), 7.35-7.16 (m, 7H), 6.61 (t, J=72.5 Hz, 1H), 5.77-5.60 (m, 1H), 5.10-4.95 (m, 1H), 4.04-3.55 (m, 5H), 3.19-2.94 (m, 3H), 2.93-2.71 (m, 2H), 1.71-1.55 (m, 1H), 1.49-1.36 (m, 1H).

### Example 11

Synthesis (3R,3aS,6aR)-hexahydrofuro[2,3-b]furan-3-yl-((2S,3R)-4-(4-cyano-N-isobutyl phenylsulfonamido)-3-hydroxy-1-phenylbutan-2-yl)carbamate-d12 (Compound 222).

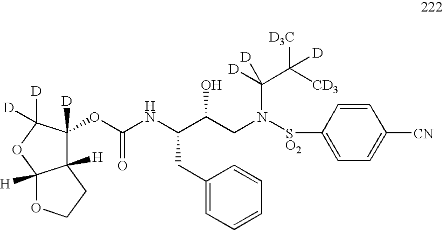

Step 1. tert-butyl-((2S,3R)-4-(4-cyano-N-isobutylphenylsulfonamido)-3-hydroxy-1-phenylbutan-2-yl)carbamate-d9 (5g). Followed step 2 for Example 5 employing amino alcohol 12a and sulfonyl chloride 13e to afford sulfonamide 14g (89%) as a white solid. MS (ESI) 411.2 [(M-Boc+H)⁺].

Step 2. (3R,3aS,6aR)-hexahydrofuro[2,3-b]furan-3-yl-((2S,3R)-4-(4-cyano-N-isobutyl phenylsulfonamido)-3-hydroxy-1-phenylbutan-2-yl)carbamate-d12 (222). Followed step 3 for Example 5 employing sulfonamide 14g and carbonate 16a to afford 222 (48%) as a white solid. MS (ESI) 570.3 [(M+H)⁺]. ¹H NMR (CDCl₃, 400 MHz, rotamers) δ 7.91-7.73 (m, 4H), 7.32-7.15 (m, 5H), 5.72-5.61 (m, 1H), 5.00-4.81 (m, 1H), 4.08-3.75 (m, 3H), 3.74-3.60 (m, 1H), 3.44 (br s, 1H), 3.19-2.89 (m, 3H), 2.89-2.70 (m, 2H), 1.98-1.71 (m, 1H), 1.71-1.55 (m, 1H), 1.49-1.36 (m, 1H).

### Example 12

Synthesis (3R,3aS,6aR)-hexahydrofuro[2,3-b]furan-3-yl-((2S,3R)-4-(N-isobutyl-4-(trifluoro-methyl)phenylsulfonamido)-3-hydroxy-1-phenylbutan-2-yl)carbamate-d12 (Compound 221).

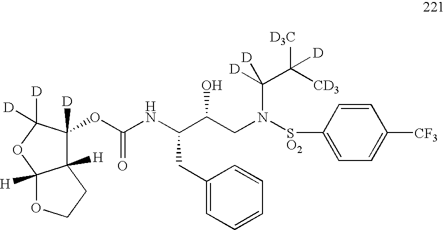

Step 1. tert-butyl-((2S,3R)-4-(N-isobutyl-4-(trifluoromethyl)phenylsulfonamido)-3-hydroxy-1-phenylbutan-2-yl)carbamate-d9 (14h). Followed step 2 for Example 5 employing amino alcohol 12a and sulfonyl chloride 13o to afford sulfonamide 14h (93%) as a white solid. MS (ESI) 454.2 [(M-Boc+H)⁺].

Step 2. (3R,3aS,6aR)-hexahydrofuro[2,3-b]furan-3-yl-((2S,3R)-4-(N-isobutyl-4-(trifluoro-methyl)phenylsulfonamido)-3-hydroxy-1-phenylbutan-2-yl)carbamate-d12 (221). Followed step 3 for Example 5 employing sulfonamide 14h and carbonate 16a to afford 221 (54%) as a white solid. MS (ESI) 454.2 [(M-BisTHFCO₂+H)⁺]. ¹H NMR (CDCl₃, 400 MHz, rotamers) δ 7.91 (d, J=8.3 Hz, 2H), 7.80 (d, J=8.6 Hz, 2H), 7.32-7.15 (m, 5H), 5.72-5.61 (m, 1H), 5.00-4.81 (m, 1H), 4.08-3.75 (m, 3H), 3.74-3.60 (m, 1H), 3.44 (br s, 1H), 3.19-2.89 (m, 3H), 2.89-2.70 (m, 2H), 1.98-1.71 (m, 1H), 1.71-1.55 (m, 1H), 1.49-1.36 (m, 1H).

### Example 13

Synthesis (3R,3aS,6aR)-hexahydrofuro[2,3-b]furan-3-yl-((2S,3R)-3-hydroxy-4-(N-isobutyl benzofuran-5-sulfonamido)-1-phenylbutan-2-yl)carbamate-d12 (Compound 106).

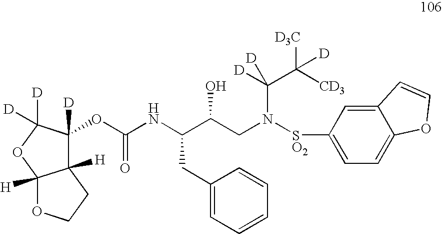

Step 1. tert-butyl-((2S,3R)-3-hydroxy-4-(N-isobutylbenzofuran-5-sulfonamido)-1-phenyl-butan-2-yl)carbamate-d9 (14i). Followed step 2 for Example 5 employing amino alcohol 12a and sulfonyl chloride 13h to afford sulfonamide 14i (88%) as a white solid. MS (ESI) 426.2 [(M-Boc+H)⁺].

Step 2. (3R,3aS,6aR)-hexahydrofuro[2,3-b]furan-3-yl-((2S,3R)-3-hydroxy-4-(N-isobutyl benzofuran-5-sulfonamido)-1-phenylbutan-2-yl)carbamate-d12 (106). Followed step 3 for Example 5 employing sulfonamide 14i and carbonate 16a to afford 106 (53%) as a white solid. MS (ESI) 585.3 [(M+H)⁺]. ¹H NMR (CDCl₃, 400 MHz, rotamers) δ 8.10 (s, 1H), 7.77 (s, 1H), 7.74-7.59 (m, 2H), 7.33-7.15 (m, 5H), 6.90 (s, 1H), 5.72-5.61 (m, 1H), 5.10-4.90 (m, 1H), 4.08-3.75 (m, 3H), 3.75-3.62 (m, 2H), 3.26-2.75 (m, 5H), 1.98-1.71 (m, 1H), 1.71-1.55 (m, 1H), 1.49-1.36 (m, 1H).

### Example 14

Synthesis (3R,3aS,6aR)-hexahydrofuro[2,3-b]furan-3-yl((2S,3R)-3-hydroxy-4-(N-isobutyl-2,3-dihydrobenzo[b][1,4]dioxine-6-sulfonamido)-1-phenylbutan-2-yl)carbamate-d12 (Compound 224).

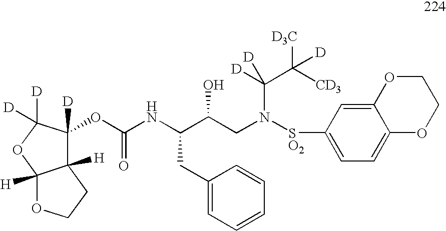

Step 1. tert-butyl-((2S,3R)-3-hydroxy-4-(N-isobutyl-2,3-dihydrobenzo[b][1,4]dioxine-6-sulfonamido)-1-phenylbutan-2-yl)carbamate-d9 (14j). Followed step 2 for Example 5 employing amino alcohol 12a and sulfonyl chloride 13f to afford sulfonamide 14j (90%) as a white solid. MS (ESI) 444.2 [(M-Boc+H)⁺].

Step 2. (3R,3aS,6aR)-hexahydrofuro[2,3-b]furan-3-yl((2S,3R)-3-hydroxy-4-(N-isobutyl-2,3-dihydrobenzo[b][1,4]dioxine-6-sulfonamido)-1-phenylbutan-2-yl)carbamate-d12 (224). Followed step 3 for Example 5 employing sulfonamide 14j and carbonate 16a to afford 224 (44%) as a white solid. MS (ESI) 603.3 [(M+H)⁺]. ¹H NMR (CDCl₃, 400 MHz, rotamers) δ 7.39-7.16 (m, 7H), 6.96 (d, J=8.6 Hz, 1H), 5.72-5.61 (m, 1H), 5.10-4.90 (m, 1H), 4.40-4.25 (m, 4H), 4.08-3.75 (m, 3H), 3.75-3.62 (m, 2H), 3.23-2.66 (m, 5H), 1.98-1.71 (m, 1H), 1.71-1.55 (m, 1H), 1.49-1.36 (m, 1H).

### Example 15

Synthesis (3R,3aS,6aR)-hexahydrofuro[2,3-b]furan-3-yl-((2S,3R)-4-(4-acetamido-N-isobutyl phenylsulfonamido)-3-hydroxy-1-phenylbutan-2-yl) carbamate-d12 (Compound 227).

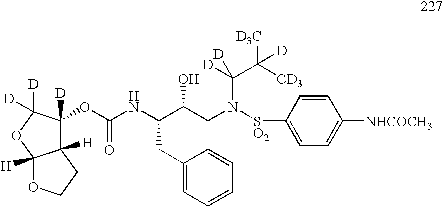

Step 1. tert-butyl-((2S,3R)-4-(4-acetamido-N-isobutylphenylsulfonamido)-3-hydroxy-1-phenylbutan-2-yl)carbamate-d9 (14k). Followed step 2 for Example 5 employing amino alcohol 12a and sulfonyl chloride 13i to afford sulfonamide 14k (89%) as a white solid. MS (ESI) 443.2 [(M-Boc+H)⁺].

Step 2. (3R,3aS,6aR)-hexahydrofuro[2,3-b]furan-3-yl-((2S,3R)-4-(4-acetamido-N-isobutyl phenylsulfonamido)-3-hydroxy-1-phenylbutan-2-yl) carbamate-d12 (227). Followed step 3 for Example 5 employing sulfonamide 14k and carbonate 16a to afford 227 (43%) as a white solid. MS (ESI) 602.3 [(M+H)⁺]. ¹H NMR (CDCl₃, 400 MHz, rotamers) δ 7.79-7.58 (m, 4H), 7.34-7.17 (m, 5H), 5.72-5.61 (m, 1H), 5.10-4.90 (m, 1H), 4.00-3.52 (m, 5H), 3.23-2.66 (m, 5H), 2.21 (s, 3H), 1.71-1.55 (m, 1H), 1.49-1.36 (m, 1H).

### Example 16

Synthesis (3R,3aS,6aR)-hexahydrofuro[2,3-b]furan-3-yl-((2S,3R)-3-hydroxy-4-(N-isobutyl benzo[d]thiazole-6-sulfonamido)-1-phenylbutan-2-yl)carbamate-d12 (Compound 229).

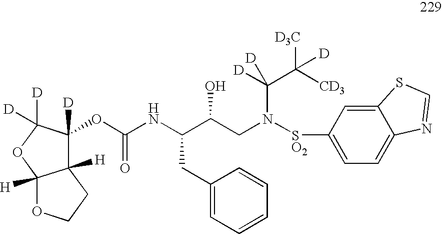

Step 1. tert-butyl-((2S,3R)-3-hydroxy-4-(N-isobutylbenzo[d]thiazole-6-sulfonamido)-1-phenylbutan-2-yl)carbamate-d9 (14-1). Followed step 2 for Example 5 employing amino alcohol 12a and sulfonyl chloride 13j to afford sulfonamide 14-1 (96%) as a white solid. MS (ESI) 443.2 [(M-Boc+H)⁺].

Step 2. (3R,3aS,6aR)-hexahydrofuro[2,3-b]furan-3-yl-((2S,3R)-3-hydroxy-4-(N-isobutyl benzo[d]thiazole-6-sulfonamido)-1-phenylbutan-2-yl)carbamate-d12 (229). Followed step 3 for Example 5 employing sulfonamide 14-1 and carbonate 16a to afford 229 (64%) as a white solid. MS (ESI) 602.3 [(M+H)⁺]. ¹H NMR (CDCl₃, 400 MHz, rotamers) δ 9.22 (s, 1H), 8.45 (s, 1H), 8.25 (d, J=8.6 Hz, 1H), 7.90 (d, J=10.6 Hz, 1H), 7.38-7.17 (m, 5H), 5.72-5.61 (m, 1H), 5.10-4.90 (m, 1H), 3.98-3.75 (m, 3H), 3.75-3.58 (m, 2H), 3.26-2.68 (m, 5H), 1.98-1.71 (m, 1H), 1.71-1.55 (m, 1H), 1.49-1.36 (m, 1H).

### Example 17

Synthesis (3R,3aS,6aR)-hexahydrofuro[2,3-b]furan-3-yl-((2S,3R)-4-(4-(cyclopropanecarbox-amido)-N-isobutylphenylsulfonamido)-3-hydroxy-1-phenylbutan-2-yl)carbamate-d12 (Compound 233).

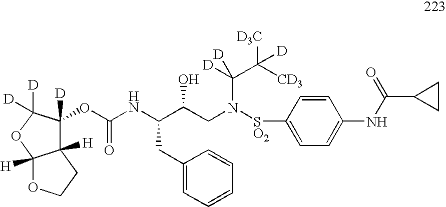

Step 1. tert-butyl-((2S,3R)-4-(4-(cyclopropanecarboxamido)-N-isobutylphenylsulfonamido)-3-hydroxy-1-phenylbutan-2-yl)carbamate-d9 (14m). Followed step 2 for Example 5 employing amino alcohol 12a and sulfonyl chloride 13m to afford sulfonamide 14m (100%) as a white solid. MS (ESI) 469.3 [(M-Boc+H)⁺].

Step 2. (3R,3aS,6aR)-hexahydrofuro[2,3-b]furan-3-yl-((2S,3R)-4-(4-(cyclopropanecarbox-amido)-N-isobutylphenylsulfonamido)-3-hydroxy-1-phenylbutan-2-yl)carbamate-d12 (233). Followed step 3 for Example 5 employing sulfonamide 14m and carbonate 16a to afford 233 (57%) as a white solid. MS (ESI) 628.3 [(M+H)⁺]. ¹H NMR (CDCl₃, 400 MHz, rotamers) δ 7.80-7.64 (m, 4H), 7.34-7.17 (m, 5H), 5.72-5.61 (m, 1H), 5.10-4.90 (m, 1H), 3.98-3.79 (m, 3H), 3.78-3.65 (m, 1H), 3.60 (s, 1H), 3.22-2.72 (m, 5H), 1.75-1.42 (m, 3H), 1.19-1.09 (m, 2H), 0.98-0.85 (m, 2H).

### Example 18

Synthesis (3R,3aS,6aR)-hexahydrofuro[2,3-b]furan-3-yl-((2S,3R)-4-(4-(cyclobutanecarbox-amido)-N-isobutylphenylsulfonamido)-3-hydroxy-1-phenylbutan-2-yl)carbamate-d12 (Compound 234).

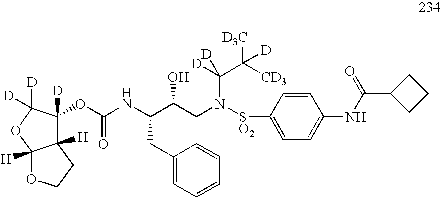

Step 1. tert-butyl-((2S,3R)-4-(4-(cyclobutanecarboxamido)-N-isobutylphenylsulfonamido)-3-hydroxy-1-phenylbutan-2-yl)carbamate-d9 (14n). Followed step 2 for Example 5 employing amino alcohol 12a and sulfonyl chloride 13n to afford sulfonamide 14n (91%) as a white solid. MS (ESI) 483.4 [(M-Boc+H)⁺].

Step 2. (3R,3aS,6aR)-hexahydrofuro[2,3-b]furan-3-yl-((2S,3R)-4-(4-(cyclobutanecarbox-amido)-N-isobutylphenylsulfonamido)-3-hydroxy-1-phenylbutan-2-yl)carbamate-d12 (234): Followed step 3 for Example 5 employing sulfonamide 14n and carbonate 16a to afford 234 (71%) as a white solid. MS (ESI) 642.3 [(M+H)⁺]. ¹H NMR (CDCl₃, 400 MHz, rotamers) δ 7.71 (s, 4H), 7.35-7.16 (m, 5H), 5.72-5.61 (m, 1H), 5.10-4.90 (m, 1H), 3.98-3.79 (m, 3H), 3.78-3.65 (m, 1H), 3.57 (s, 1H), 3.25-2.72 (m, 6H), 2.48-2.34 (m, 2H), 2.32-2.19 (m, 2H), 2.11-1.88 (m, 3H), 1.71-1.56 (m, 1H), 1.50-1.42 (m, 1H).

### Example 19

Synthesis (3R,3aS,6aR)-hexahydrofuro[2,3-b]furan-3-yl42S,3R)-3-hydroxy-4-(N-isobutyl-2,3-dihydrobenzofuran-5-sulfonamido)-1-phenylbutan-2-yl)carbamate-d12 (Compound 223).

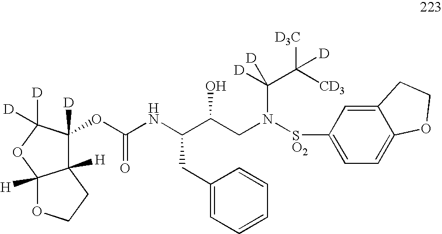

Step 1. tert-butyl-((2S,3R)-3-hydroxy-4-(N-isobutyl-2,3-dihydrobenzofuran-5-sulfonamido)-1-phenylbutan-2-yl)carbamate-d9 (14o). Followed step 2 for Example 5 employing amino alcohol 12a and sulfonyl chloride 13g to afford sulfonamide 14o (50%) as a white solid. MS (ESI) 428.3 [(M-Boc+H)⁺].

Step 2. (3R,3aS,6aR)-hexahydrofuro[2,3-b]furan-3-yl((2S,3R)-3-hydroxy-4-(N-isobutyl-2,3-dihydrobenzofuran-5-sulfonamido)-1-phenylbutan-2-yl)carbamate-d12 (223). Followed step 3 for Example 5 employing sulfonamide 14o and carbonate 16a to afford 223 (55%) as a white solid. MS (ESI) 587.3 [(M+H)⁺]. ¹H NMR (CDCl₃, 400 MHz, rotamers) δ 7.62-7.52 (m, 2H), 7.34-7.16 (m, 5H), 6.85 (d, J=8.6 Hz, 1H), 5.78-5.61 (m, 1H), 5.00-4.88 (m, 1H), 4.69 (t, J=8.8 Hz, 2H), 3.98-3.75 (m, 3H), 3.76-3.62 (m, 2H), 3.28 (t, J=8.8 Hz, 2H), 3.21-2.74 (m, 5H), 1.98-1.71 (m, 1H), 1.71-1.55 (m, 1H), 1.49-1.36 (m, 1H).

### Example 20

Synthesis (3R,3aS,6aR)-hexahydrofuro[2,3-b]furan-3-yl-((2S,3R)-3-hydroxy-4-(N-isobutyl benzo[d][1,3]dioxole-5-sulfonamido)-1-phenylbutan-2-yl)carbamate-d18 (Compound 238).

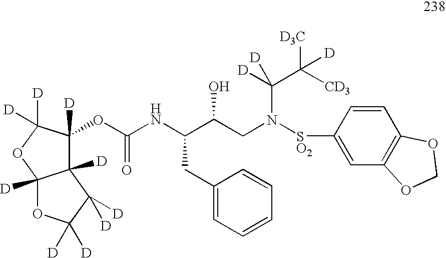

Step 1. tert-butyl-((2S,3R)-3-hydroxy-4-(N-isobutylbenzo[d][1,3]dioxole-5-sulfonamido)-1-phenylbutan-2-yl)carbamate-d9 (14p). Followed step 2 for Example 5 employing 10% NaHCO₃ as base in place of N,N-diisopropylethylamine, amino alcohol 12a and sulfonyl chloride 13p to afford sulfonamide 14p (92%) as a white solid. MS (ESI) 430.3 [(M-Boc+H)⁺].

Step 2. (3R,3aS,6aR)-hexahydrofuro[2,3-b]furan-3-yl-((2S,3R)-3-hydroxy-4-(N-isobutyl benzo[d][1,3]dioxole-5-sulfonamido)-1-phenylbutan-2-yl)carbamate-d18 (238). Followed step 3 for Example 5 employing sulfonamide 14p and carbonate 16c to afford 238 (76%) as a white solid. MS (ESI) 595.3 [(M+H)⁺]. ¹H NMR (CDCl₃, 400 MHz, rotamers) δ 7.39-7.15 (m, 7H), 6.90 (d, J=8.3 Hz, 1H), 6.09 (s, 2H), 4.96 (d, J=8.8 Hz, 1H), 3.98-3.79 (m, 2H), 3.63 (s, 1H), 3.19-2.94 (m, 3H), 2.85-2.73 (m, 1H).

### Example 21

Synthesis (3R,3aS,6aR)-hexahydrofuro[2,3-b]furan-3-yl-((2S,3R)-4-(4-acetamido-N-(cyclopen-tylmethyl)phenylsulfonamido)-3-hydroxy-1-phenylbutan-2-yl)carbamate-d14 (Compound 236).

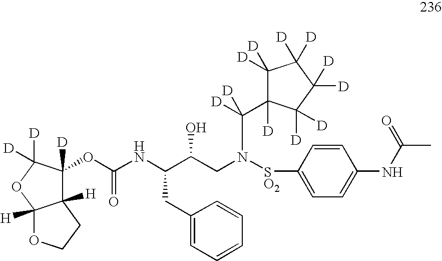

Step 1. tert-butyl-((2S,3R)-4-((cyclopentylmethyl)amino)-3-hydroxy-1-phenylbutan-2-yl)carbamate-d11 (12b): Followed step 1 for Example 5 employing epoxide 10a and amine 11b to afford amino alcohol 12b (65%). MS (ESI) 374.4 [(M+H)⁺].

Step 2. tert-butyl-((2S,3R)-4-(4-acetamido-N-(cyclopentylmethyl)phenylsulfonamido)-3-hydroxy-1-phenylbutan-2-yl)carbamate-d11 (14q): Followed step 2 for Example 5 employing 10% NaHCO₃ as base in place of N,N-diisopropylethylamine, amino alcohol 12b and sulfonyl chloride 13i to afford sulfonamide 14q (100%) as a white solid. MS (ESI) 471.3 [(M-Boc+H)⁺].

Step 3. (3R,3aS,6aR)-hexahydrofuro[2,3-b]furan-3-yl-((2S,3R)-4-(4-acetamido-N-(cyclopen-tylmethyl)phenylsulfonamido)-3-hydroxy-1-phenylbutan-2-yl)carbamate-d14 (236): Followed step 3 for Example 5 employing sulfonamide 14q and carbonate 16a to afford 236 (80%) as a white solid. MS (ESI) 630.3 [(M+H)⁺]. ¹H NMR (CDCl₃, 400 MHz, rotamers) δ 7.79-7.58 (m, 4H), 7.34-7.17 (m, 5H), 5.72-5.61 (m, 1H), 5.10-4.90 (m, 1H), 4.00-3.52 (m, 5H), 3.23-2.66 (m, 5H), 2.21 (s, 3H), 1.71-1.55 (m, 1H), 1.49-1.36 (m, 1H).

### Example 22

Synthesis (3R,3aS,6aR)-hexahydrofuro[2,3-b]furan-3-yl-((2S,3R)-4-(4-acetamido-N-(cyclopen-tylmethyl)phenylsulfonamido)-3-hydroxy-1-phenylbutan-2-yl)carbamate-d20 (Compound 228).

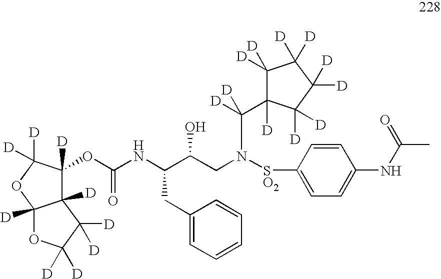

(3R,3aS,6aR)-hexahydrofuro[2,3-b]furan-3-yl-((2S,3R)-4-(4-acetamido-N-(cyclopen-tylmethyl)phenylsulfonamido)-3-hydroxy-1-phenylbutan-2-yl)carbamate-d20 (228): Followed step 3 for Example 5 employing sulfonamide 14q and carbonate 16c to afford 228 (70%) as a white solid. MS (ESI) 636.4 [(M+H)⁺]. ¹H NMR (CDCl₃, 400 MHz, rotamers) δ 7.79-7.58 (m, 4H), 7.34-7.17 (m, 5H), 4.97 (d, J=8.8 Hz, 1H), 3.89 (br s, 2H), 3.53 (s, 1H), 3.19-2.97 (m, 3H), 2.86-2.72 (m, 1H), 2.21 (s, 3H).

### Example 23

Synthesis (3R,3aS,6aR)-hexahydrofuro[2,3-b]furan-3-yl-((2S,3R)-3-hydroxy-4-(N-isobutyl benzo[d]thiazole-6-sulfonamido)-1-phenylbutan-2-yl)carbamate-d16 (Compound 237).

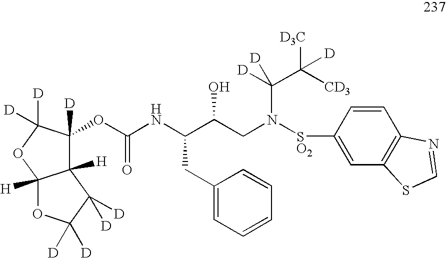

(3R,3aS,6aR)-hexahydrofuro[2,3-b]furan-3-yl-((2S,3R)-3-hydroxy-4-(N-isobutyl benzo[d]thiazole-6-sulfonamido)-1-phenylbutan-2-yl)carbamate-d16 (237): Followed step 3 for Example 5 employing sulfonamide 14-1 and carbonate 16b to afford 237 (62%) as a white solid. MS (ESI) 606.3 [(M+H)⁺]. ¹H NMR (CDCl₃, 400 MHz, rotamers) δ 9.22 (s, 1H), 8.45 (s, 1H), 8.25 (d, J=8.6 Hz, 1H), 7.90 (d, J=10.6 Hz, 1H), 7.38-7.17 (m, 5H), 5.63 (d, J=5.1 Hz, 1H), 4.99 (d, J=8.3 Hz, 1H), 3.89 (br s, 2H), 3.65 (s, 1H), 3.26-3.16 (m, 1H), 3.14-2.98 (m, 2H), 2.90-2.74 (m, 2H).

### Example 24

Synthesis (3R,3aS,6aR)-hexahydrofuro[2,3-b]furan-3-yl-((2S,3R)-3-hydroxy-4-(N-isobutyl benzo[d]thiazole-6-sulfonamido)-1-phenylbutan-2-yl)carbamate-d18 (Compound 230).

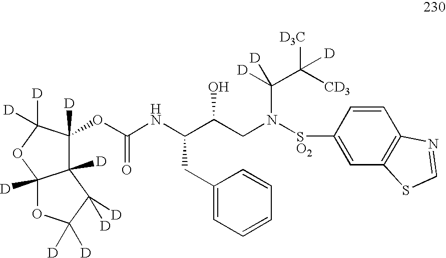

(3R,3aS,6aR)-hexahydrofuro[2,3-b]furan-3-yl-((2S,3R)-3-hydroxy-4-(N-isobutyl benzo[d]thiazole-6-sulfonamido)-1-phenylbutan-2-yl)carbamate-d18 (230): Followed step 3 for Example 5 employing sulfonamide 14-1 and carbonate 16c to afford 230 (65%) as a white solid. MS (ESI) 608.3 [(M+H)⁺]. ¹H NMR (CDCl₃, 400 MHz, rotamers) δ 9.22 (s, 1H), 8.45 (s, 1H), 8.25 (d, J=8.6 Hz, 1H), 7.90 (d, J=10.6 Hz, 1H), 7.38-7.17 (m, 5H), 4.99 (d, J=8.3 Hz, 1H), 3.89 (br s, 2H), 3.65 (s, 1H), 3.26-3.16 (m, 1H), 3.14-2.98 (m, 2H), 2.90-2.74 (m, 1H).

### Example 25

Synthesis (3R,3aS,6aR)-hexahydrofuro[2,3-b]furan-3-yl-((2S,3R)-4-(3,4-difluoro-N-isobutyl-phenylsulfonamido)-3-hydroxy-1-(4-methoxyphenyl)butan-2-yl)carbamate-d15 (Compound 208).

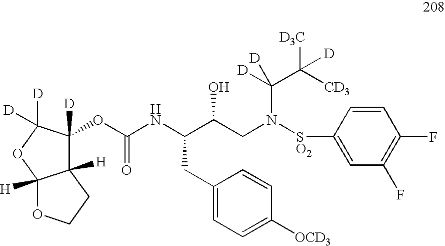

Step 1. tert-butyl-((2S,3R)-3-hydroxy-4-(isobutylamino)-1-(4-methoxypenyl)butan-2-yl)carbamate-d12 (12c): Followed step 1 for Example 5 employing epoxide 10b and the hydrochloride salt of amine 11a with the addition of N,N-diisopropylethylamine to afford amino alcohol 12c (51%). MS (ESI) 379.4 [(M+H)⁺].

Step 2. tert-butyl-((2S,3R)-4-(3,4-difluoro-N-isobutylphenylsulfonamido)-3-hydroxy-1-(4-methoxyphenyl)butan-2-yl)carbamate-d12 (14r): Followed step 2 for Example 5 employing amino alcohol 12c and sulfonyl chloride 13b to afford sulfonamide 14r (30%) as a white solid. MS (ESI) 454.9 [(M-Boc+H)⁺].

Step 3. (3R,3aS,6aR)-hexahydrofuro[2,3-b]furan-3-yl-((2S,3R)-4-(3,4-difluoro-N-isobutyl-phenylsulfonamido)-3-hydroxy-1-(4-methoxyphenyl)butan-2-yl)carbamate-d15 (208): Followed step 3 for Example 5 employing sulfonamide 14r and carbonate 16a to afford 208 (63%) as a white solid. MS (ESI) 613.9 [(M+H)⁺]. ¹H NMR (CDCl₃, 400 MHz, rotamers) δ 7.68-7.50 (m, 2H), 7.38-7.27 (m, 1H), 7.17-7.06 (m, 2H), 6.86-6.77 (m, 2H), 5.72-5.61 (m, 1H), 5.10-4.90 (m, 1H), 4.00-3.75 (m, 3H), 3.75-3.60 (m, 1H), 3.49 (s, 1H), 3.20-2.65 (m, 5H), 1.94-1.81 (m, 1H), 1.71-1.55 (m, 1H), 1.49-1.37 (m, 1H).

### Example 26

Synthesis (3R,3aS,6aR)-hexahydrofuro[2,3-b]furan-3-yl-((2S,3R)-4-(3-fluoro-N-isobutyl-4-methoxyphenylsulfonamido)-3-hydroxy-1-(4-methoxyphenyl)butan-2-yl)carbamate-d18 (Compound 204).

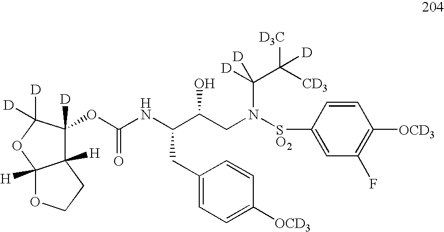

Step 1. tert-butyl((2S,3R)-4-(3-fluoro-N-isobutyl-4-methoxyphenylsulfonamido)-3-hydroxy-1-(4-metho xyphenyl)butan-2-yl)carb amate-d15 (14s): Followed step 2 for Example 5 employing amino alcohol 12c and sulfonyl chloride 134 to afford sulfonamide 14s (95%) as a white solid. MS (ESI) 470.4 [(M-Boc+H)⁺].

Step 2. (3R,3aS,6aR)-hexahydrofuro[2,3-b]furan-3-yl-((2S,3R)-4-(3-fluoro-N-isobutyl-4-methoxyphenylsulfonamido)-3-hydroxy-1-(4-methoxyphenyl)butan-2-yl)carbamate-d18 (204): Followed step 3 for Example 5 employing sulfonamide 14s and carbonate 16a to afford 204 (74%) as a white solid. MS (ESI) 629.3 [(M+H)⁺]. ¹H NMR (CDCl₃, 400 MHz, rotamers) δ 7.57-7.45 (m, 2H), 7.12 (d, J=8.6 Hz, 2H), 7.04 (t, J=8.3 Hz, 1H), 6.81 (d, J=8.6 Hz, 2H), 5.64 (d, J=5.3 Hz, 1H), 4.97 (d, J=8.6 Hz, 1H), 3.90-3.75 (m, 3H), 3.75-3.60 (m, 1H), 3.57 (s, 1H), 3.19-2.88 (m, 4H), 2.79-2.66 (m, 1H), 1.71-1.55 (m, 1H), 1.49-1.37 (m, 1H).

### Example 27

Synthesis (3R,3aS,6aR)-hexahydrofuro[2,3-b]furan-3-yl-((2S,3R)-4-(N-isobutyl-4-methoxy-phenylsulfonamido)-3-hydroxy-1-(4-methoxyphenyl)butan-2-yl)carbamate-d18 (Compound 200).

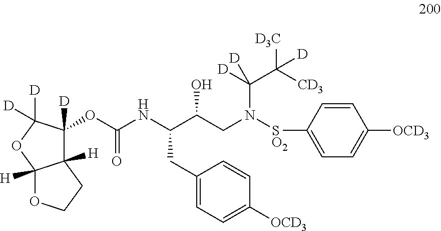

Step 1. tert-butyl-((2S,3R)-4-(N-isobutyl-4-methoxyphenylsulfonamido)-3-hydroxy-1-(4-methoxyphenyl)butan-2-yl)carbamate-d15 (14t): Followed step 2 for Example 5 employing amino alcohol 12c and sulfonyl chloride 131 to afford sulfonamide 14t (70%) as a white solid. MS (ESI) 452.4 [(M-Boc+H)⁺].

Step 2. (3R,3aS,6aR)-hexahydrofuro[2,3-b]furan-3-yl-((2S,3R)-4-(N-isobutyl-4-methoxy-phenylsulfonamido)-3-hydroxy-1-(4-methoxyphenyl)butan-2-yl)carbamate-d18 (200): Followed step 3 for Example 5 employing sulfonamide 14t and carbonate 16a to afford 200 (77%) as a white solid. MS (ESI) 611.3 [(M+H)⁺]. ¹H NMR (CDCl₃, 400 MHz, rotamers) δ 7.70 (d, J=8.8 Hz, 2H), 7.13 (d, J=8.3 Hz, 2H), 6.98 (d, J=8.8 Hz, 2H), 6.81 (d, J=8.6 Hz, 2H), 5.65 (d, J=5.1 Hz, 1H), 4.93 (d, J=8.8 Hz, 1H), 3.93-3.75 (m, 3H), 3.74-3.64 (m, 2H), 3.20-3.05 (m, 1H), 3.04-2.84 (m, 3H), 2.80-2.69 (m, 1H), 1.71-1.55 (m, 1H), 1.51-1.40 (m, 1H).

### Example 28

Synthesis (3R,3aS,6aR)-hexahydrofuro[2,3-b]furan-3-yl-((2S,3R)-3-hydroxy-4-(N-isobutyl-4-(trifluoromethoxy)phenylsulfonamido)-1-(4-methoxyphenyl)butan-2-yl)carbamate-d15 (Compound 215).

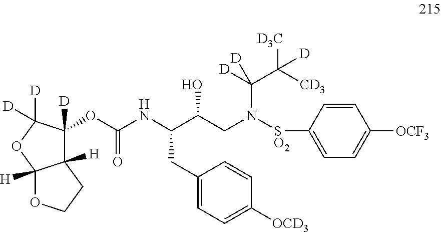

Step 1. tert-butyl-((2S,3R)-3-hydroxy-4-(N-isobutyl-4-(trifluoromethoxy)phenylsulfon-amido)-1-(4-methoxyphenyl)butan-2-yl)carbamate-d12 (14u): Followed step 2 for Example 5 employing amino alcohol 12c and sulfonyl chloride 13c to afford sulfonamide 14u (81%) as a white solid. MS (ESI) 503.3 [(M-Boc+H)⁺].

Step 2. (3R,3aS,6aR)-hexahydrofuro[2,3-b]furan-3-yl-((2S,3R)-3-hydroxy-4-(N-isobutyl-4-(trifluoromethoxy)phenylsulfonamido)-1-(4-methoxyphenyl)butan-2-yl)carbamate-d15 (215): Followed step 3 for Example 5 employing sulfonamide 14u and carbonate 16a to afford 215 (53%) as a white solid. MS (ESI) 662.2 [(M+H)⁺]. ¹H NMR (CDCl₃, 400 MHz, rotamers) δ 7.84 (d, J=8.8 Hz, 2H), 7.37 (d, J=8.8 Hz, 2H), 7.15 (d, J=7.8 Hz, 2H), 6.89-6.81 (m, 2H), 5.75-5.65 (m, 1H), 5.07-4.92 (m, 1H), 4.06-3.78 (m, 3H), 3.78-3.66 (m, 1H), 3.58 (s, 1H), 3.25-2.70 (m, 5H), 2.38-2.28 (m, 0.4H), 1.96-1.81 (0.4H), 1.77-1.61 (m, 0.6H), 1.59-1.46 (m, 0.6H).

### Example 29

Synthesis (3R,3aS,6aR)-hexahydrofuro[2,3-b]furan-3-yl-((2S,3R)-3-hydroxy-4-(N-isobutyl-4-(difluoromethoxy)phenylsulfonamido)-1-(4-methoxyphenyl)butan-2-yl)carbamate-d15 (Compound 212).

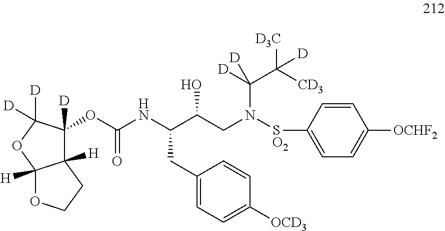

Step 1. tert-butyl-((2S,3R)-3-hydroxy-4-(N-isobutyl-4-(difluoromethoxy)phenylsulfon-amido)-1-(4-methoxyphenyl)butan-2-yl)carbamate-d12 (14v): Followed step 2 for Example 5 employing amino alcohol 12c and sulfonyl chloride 13d to afford sulfonamide 14v (81%) as a white solid. MS (ESI) 485.3 [(M-Boc+H)⁺].

Step 2. (3R,3aS,6aR)-hexahydrofuro[2,3-b]furan-3-yl-((2S,3R)-3-hydroxy-4-(N-isobutyl-4-(difluoromethoxy)phenylsulfonamido)-1-(4-methoxyphenyl)butan-2-yl)carbamate-d15 (212): Followed step 3 for Example 5 employing sulfonamide 14v and carbonate 16a to afford 212 (55%) as a white solid. MS (ESI) 644.5 [(M+H)⁺]. ¹H NMR (CDCl₃, 400 MHz, rotamers) δ 7.78 (d, J=7.8 Hz, 2H), 7.24 (d, J=8.1 Hz, 2H), 7.12 (d, J=7.8 Hz, 2H), 6.88-6.77 (m, 2H), 6.61 (t, J=72.3 Hz, 1H), 5.72-5.61 (m, 1H), 5.05-4.91 (m, 1H), 4.03-3.75 (m, 3H), 3.75-3.64 (m, 1H), 3.56 (s, 1H), 3.20-3.06 (m, 1H), 3.05-2.80 (m, 3H), 2.80-2.67 (m, 1H), 2.35-2.25 (m, 0.3H), 1.96-1.78 (0.3H), 1.74-1.59 (m, 0.7H), 1.56-1.45 (m, 0.7H).

### Example 30

Synthesis (3R,3aS,6aR)-hexahydrofuro[2,3-b]furan-3-yl-((2S,3R)-4-(3,4-difluoro-N-isobutyl-phenylsulfonamido)-3-hydroxy-1-(4-isopropoxyphenyl)butan-2-yl)carbamate-d19 (Compound 210).

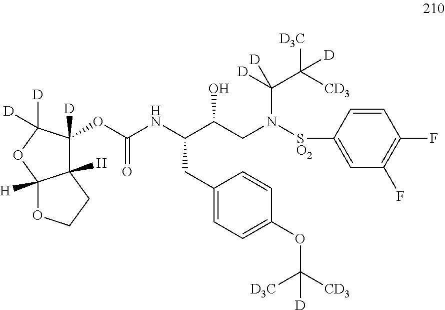

Step 1. tert-butyl-((2S,3R)-3-hydroxy-4-(isobutylamino)-1-(4-isopropoxyphenyl)butan-2-yl)carbamate-d16 (12d): Followed step 1 for Example 5 employing epoxide 10c and the hydrochloride salt of amine 11a with the addition of N,N-diisopropylethylamine to afford amino alcohol 12d (48%). MS (ESI) 411.4 [(M+H)⁺].

Step 2. tert-butyl-((2S,3R)-4-(3,4-difluoro-N-isobutylphenylsulfonamido)-3-hydroxy-1-(4-isopropoxyphenyl)butan-2-yl)carbamate-d16 (14w): Followed step 2 for Example 5 employing amino alcohol 12d and sulfonyl chloride 13b to afford sulfonamide 14w (71%) as a white solid.

Step 3. (3R,3aS,6aR)-hexahydrofuro[2,3-b]furan-3-yl-((2S,3R)-4-(3,4-difluoro-N-isobutyl-phenylsulfonamido)-3-hydroxy-1-(4-isopropoxyphenyl)butan-2-yl)carbamate-d19 (210): Followed step 3 for Example 5 employing sulfonamide 14w and carbonate 16a to afford 210 (81%) as a white solid. MS (ESI) 646.4 [(M+H)⁺]. ¹H NMR (CDCl₃, 400 MHz, rotamers) δ 7.68-7.58 (m, 1H), 7.58-7.50 (m, 1H), 7.38-7.27 (m, 1H), 7.10 (d, J=8.3 Hz, 2H), 6.86-6.77 (m, 2H), 5.72-5.59 (m, 1H), 5.10-4.87 (m, 1H), 4.00-3.60 (m, 4H), 3.47 (s, 1H), 3.20-2.79 (m, 4H), 2.78-2.66 (m, 1H), 1.94-1.63 (m, 1H), 1.58-1.47 (m, 1H).

### Example 31

Synthesis (3R,3aS,6aR)-hexahydrofuro[2,3-b]furan-3-yl-((2S,3R)-4-(3-fluoro-N-isobutyl-4-methoxyphenylsulfonamido)-3-hydroxy-1-(4-isopropoxyphenyl)butan-2-yl)-carbamate-d22 (Compound 206).

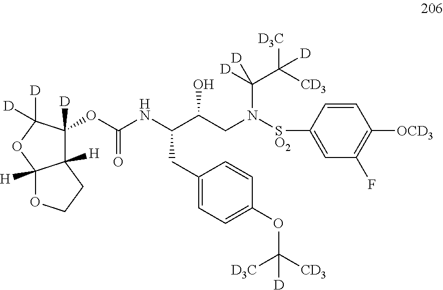

Step 1. tert-butyl 42S,3R)-4-(3-fluoro-N-isobutyl-4-methoxyphenylsulfonamido)-3-hydroxy-1-(4-isopropoxyphenyl)butan-2-yl)carbamate-d19 (14×): Followed step 2 for Example 5 employing amino alcohol 12d and sulfonyl chloride 134 to afford sulfonamide 14x (80%) as a white solid. MS (ESI) 502.4 [(M-Boc+H)⁺].

Step 2. (3R,3aS,6aR)-hexahydrofuro[2,3-b]furan-3-yl-((2S,3R)-4-(3-fluoro-N-isobutyl-4-methoxyphenylsulfonamido)-3-hydroxy-1-(4-isopropoxyphenyl)butan-2-yl)-carbamate-d22 (206): Followed step 3 for Example 5 employing sulfonamide 14x and carbonate 16a to afford 206 (77%) as a white solid. MS (ESI) 661.3 [(M+H)⁺]. ¹H NMR (CDCl₃, 400 MHz, rotamers) δ 7.59-7.43 (m, 2H), 7.16-6.98 (m, 3H), 6.84-6.74 (m, 2H), 5.72-5.62 (m, 1H), 5.04-4.89 (m, 1H), 4.08-3.63 (m, 4H), 3.58 (s, 1H), 3.20-2.78 (m, 4H), 2.78-2.66 (m, 1H), 2.37-2.26 (m, 0.3H), 1.94-1.77 (m, 0.4H), 1.73-1.58 (m, 0.7H), 1.54-1.45 (m, 0.6H).

### Example 32

Synthesis (3R,3aS,6aR)-hexahydrofuro[2,3-b]furan-3-yl-((2S,3R)-3-hydroxy-4-(N-isobutyl-4-methoxyphenylsulfonamido)-1-(4-isopropoxyphenyl)butan-2-yl)carbamate-d22 (Compound 202).

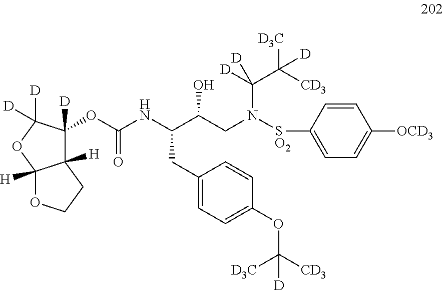

Step 1. tert-butyl-((2S,3R)-3-hydroxy-4-(N-isobutyl-4-methoxyphenylsulfonamido)-1-(4-isopropoxyphenyl)butan-2-yl)carbamate-d19 (14y): Followed step 2 for Example 5 employing amino alcohol 12d and sulfonyl chloride 131 to afford sulfonamide 14y (79%) as a white solid. MS (ESI) 484.4 [(M-Boc+H)⁺].

Step 3:(3R,3aS,6aR)-hexahydrofuro[2,3-b]furan-3-yl-((2S,3R)-3-hydroxy-4-(N-isobutyl-4-methoxyphenylsulfonamido)-1-(4-isopropoxyphenyl)butan-2-yl)carbamate-d22 (202): Followed step 3 for Example 5 employing sulfonamide 14y and carbonate 16a to afford 202 (74%) as a white solid. MS (ESI) 643.5 [(M+H)⁺]. ¹H NMR (CDCl₃, 400 MHz, rotamers) δ 7.77-7.62 (m, 2H), 7.17-7.05 (m, H), 7.03-6.93 (m, 2H), 6.86-6.73 (m, 2H), 5.71-5.60 (m, 1H), 5.07-4.89 (m, 1H), 4.03-3.75 (m, 4H), 3.75-3.64 (m, 1H), 3.20-2.79 (m, 4H), 2.79-2.68 (m, 1H), 2.37-2.26 (m, 0.3H), 1.94-1.77 (m, 0.4H), 1.73-1.58 (m, 0.7H), 1.54-1.45 (m, 0.6H).

### Example 33

Synthesis (3R,3aS,6aR)-hexahydrofuro[2,3-b]furan-3-yl-((2S,3R)-3-hydroxy-4-(N-isobutyl-4-(trifluoromethoxy)phenylsulfonamido)-1-(4-isopropoxyphenyl)butan-2-yl)-carbamate-d19 (Compound 218).

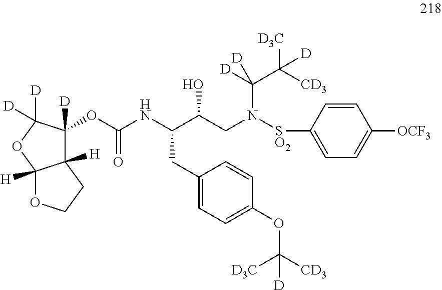

Step 1. tert-butyl-((2S,3R)-3-hydroxy-4-(N-isobutyl-4-(trifluoromethoxy)phenylsulfon-amido)-1-(4-isopropoxyphenyl)butan-2-yl)carbamate-d16 (14z): Followed step 2 for Example 5 employing amino alcohol 12d and sulfonyl chloride 13c to afford sulfonamide 14z (78%) as a white solid.

Step 2. (3R,3aS,6aR)-hexahydrofuro[2,3-b]furan-3-yl-((2S,3R)-3-hydroxy-4-(N-isobutyl-4-(trifluoromethoxy)phenylsulfonamido)-1-(4-isopropoxyphenyl)butan-2-yl)-carbamate-d19 (218): Followed step 3 for Example 5 employing sulfonamide 14z and carbonate 16a to afford 218 (43%) as a white solid. MS (ESI) 694.2 [(M+H)⁺]. ¹H NMR (CDCl₃, 400 MHz, rotamers) δ 7.81 (d, J=9.1 Hz, 2H), 7.34 (d, J=8.1 Hz, 2H), 7.10 (d, J=8.3 Hz, 2H), 6.79 (d, J=8.6 Hz, 2H), 5.77-5.61 (m, 1H), 5.06-4.89 (m, 1H), 4.04-3.60 (m, 4H), 3.55 (s, 1H), 3.22-3.07 (m, 1H), 3.07-2.79 (m, 3H), 2.79-2.67 (m, 1H), 2.36-2.23 (m, 0.2H), 1.95-1.80 (m, 0.4H), 1.74-1.60 (m, 0.8H), 1.58-1.48 (m, 0.6H).

### Example 34

Synthesis (3R,3aS,6aR)-hexahydrofuro[2,3-b]furan-3-yl-((2S,3R)-3-hydroxy-4-(N-isobutyl-4-(difluoromethoxy)phenylsulfonamido)-1-(4-isopropoxyphenyl)butan-2-yl)-carbamate-d19 (Compound 214).

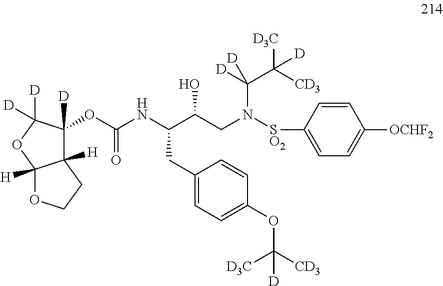

Step 1. tert-butyl-((2S,3R)-3-hydroxy-4-(N-isobutyl-4-(difluoromethoxy)phenylsulfon-amido)-1-(4-isopropoxyphenyl)butan-2-yl)carbamate-d16 (14aa): Followed step 2 for Example 5 employing amino alcohol 12d and sulfonyl chloride 13d to afford sulfonamide 14aa (75%) as a white solid. MS (ESI) 517.3 [(M-Boc+H)⁺].

Step 3:(3R,3aS,6aR)-hexahydrofuro[2,3-b]furan-3-yl-((2S,3R)-3-hydroxy-4-(N-isobutyl-4-(difluoromethoxy)phenylsulfonamido)-1-(4-isopropoxyphenyl)butan-2-yl)-carbamate-d19 (214): Followed step 3 for Example 5 employing sulfonamide 14aa and carbonate 16a to afford 214 (71%) as a white solid. MS (ESI) 676.4 [(M+H)⁺]. ¹H NMR (CDCl₃, 400 MHz, rotamers) δ 7.78 (d, J=8.6 Hz, 2H), 7.24 (d, J=8.6 Hz, 2H), 7.10 (d, J=8.3 Hz, 2H), 6.79 (d, J=8.3 Hz, 2H), 6.62 (t, J=72.8 Hz, 1H), 5.77-5.61 (m, 1H), 5.06-4.89 (m, 1H), 4.04-3.75 (m, 3H), 3.75-3.65 (m, 1H), 3.57 (s, 1H), 3.20-3.05 (m, 1H), 3.04-2.79 (m, 3H), 2.79-2.68 (m, 1H), 2.36-2.25 (m, 0.3H), 1.95-1.80 (m, 0.3H), 1.74-1.60 (m, 0.7H), 1.58-1.48 (m, 0.7H).

### Example 35

Synthesis (3R,3aS,6aR)-hexahydrofuro[2,3-b]furan-3-yl-((2S,3R)-1-(4-(benzyloxy)phenyl)-4-(3,4-difluoro-N-isobutylphenylsulfonamido)-3-hydroxybutan-2-yl)carbamate-d14 (Compound 209).

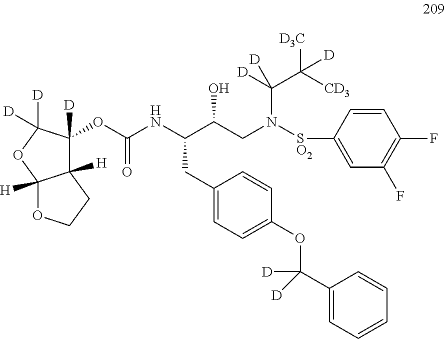

Step 1. tert-butyl-((2S,3R)-1-(4-(benzyloxy)phenyl)-3-hydroxy-4-(isobutylamino)butan-2-yl)carbamate-d11 (12e): Followed step 1 for Example 5 employing epoxide 10d and the hydrochloride salt of amine 11a with the addition of N,N-diisopropylethylamine to afford amino alcohol 12e (47%). MS (ESI) 454.0 [(M+H)⁺].

Step 2. tert-butyl-((2S,3R)-1-(4-(benzyloxy)phenyl)-4-(3,4-difluoro-N-isobutylphenylsulfon-amido)-3-hydroxybutan-2-yl)carbamate-d11 (14bb) : Followed step 2 for Example 5 employing amino alcohol 12e and sulfonyl chloride 13b to afford sulfonamide 14bb (78%) as a white solid.

Step 3. (3R,3aS,6aR)-hexahydrofuro[2,3-b]furan-3-yl-((2S,3R)-1-(4-(benzyloxy)phenyl)-4-(3,4-difluoro-N-isobutylphenylsulfonamido)-3-hydroxybutan-2-yl)carbamate-d14 (209): Followed step 3 for Example 5 employing sulfonamide 14bb and carbonate 16a to afford 209 (66%) as a white solid. MS (ESI) 530.2 [(M-BisTHFCO₂+H)⁺]. ¹H NMR (CDCl₃, 400 MHz, rotamers) δ 7.68-7.58 (m, 1H), 7.58-7.51 (m, 1H), 7.46-7.27 (m, 6H), 7.17-7.08 (m, 2H), 6.96-6.87 (m, 2H), 5.71-5.61 (m, 1H), 5.03-4.87 (m, 1H), 4.00-3.60 (m, 4H), 3.48 (s, 1H), 3.20-2.68 (m, 5H), 1.99-1.73 (m, 0.7H), 1.71-1.44 (m, 1.3H).

### Example 36

Synthesis (3R,3aS,6aR)-hexahydrofuro[2,3-b]furan-3-yl-((2S,3R)-1-(4-(benzyloxy)phenyl)-4-(3-fluoro-N-isobutyl-4-methoxyphenylsulfonamido)-3-hydroxybutan-2-yl)-carbamate-d17 (Compound 205).

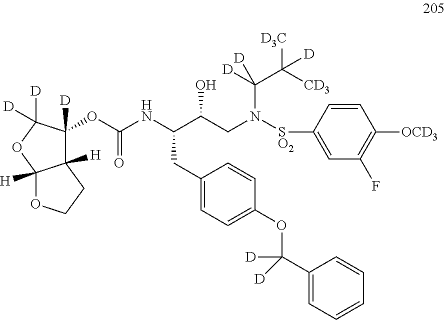

Step 1. tert-Butyl-((2S,3R)-1-(4-(benzyloxy)phenyl)-4-(3-fluoro-N-isobutyl-4-methoxyphen-ylsulfonamido)-3-hydroxybutan-2-yl)carbamate-d14 (14 cc): Followed step 2 for Example 5 employing amino alcohol 12e and sulfonyl chloride 134 to afford sulfonamide 14cc (78%) as a white solid.

Step 2. (3R,3aS,6aR)-hexahydrofuro[2,3-b]furan-3-yl-((2S,3R)-1-(4-(benzyloxy)phenyl)-4-(3-fluoro-N-isobutyl-4-methoxyphenylsulfonamido)-3-hydroxybutan-2-yl)-carbamate-d17 (205): Followed step 3 for Example 5 employing sulfonamide 14 cc and carbonate 16a to afford 205 (74%) as a white solid. MS (ESI) 704.5 [(M+H)⁺]. ¹H NMR (CDCl₃, 400 MHz, rotamers) δ 7.59-7.40 (m, 7H), 7.13 (d, J=8.3 Hz, 2H), 7.04 (t, J=8.3 Hz, 1H), 6.90 (d, J=8.6 Hz, 2H), 5.72-5.63 (m, 1H), 4.93 (d, J=7.3 Hz, 1H), 3.93-3.77 (m, 3H), 3.75-3.63 (m, 1H), 3.57 (s, 1H), 3.17-3.05 (m, 1H), 3.05-2.83 (m, 3H), 2.82-2.69 (m, 1H), 1.71-1.45 (m, 2H).

### Example 37

Synthesis (3R,3aS,6aR)-hexahydrofuro[2,3-b]furan-3-yl-((2S,3R)-1-(4-(benzyloxy)phenyl)-3-hydroxy-4-(N-isobutyl-4-methoxyphenylsulfonamido)butan-2-yl)carbamate-d17 (Compound 201).

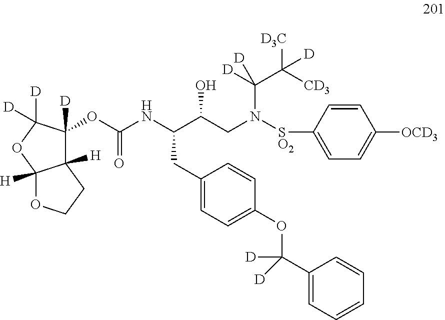

Step 1. tert-butyl-((2S,3R)-1-(4-(benzyloxy)phenyl)-3-hydroxy-4-(N-isobutyl-4-methoxy-phenylsulfonamido)butan-2-yl)carbamate-d14 (14dd): Followed step 2 for Example 5 employing amino alcohol 12e and sulfonyl chloride 131 to afford sulfonamide 14dd (74%) as a white solid.

Step 2. (3R,3aS,6aR)-hexahydrofuro[2,3-b]furan-3-yl-((2S,3R)-1-(4-(benzyloxy)phenyl)-3-hydroxy-4-(N-isobutyl-4-methoxyphenylsulfonamido)butan-2-yl)carbamate-d17 (201): Followed step 3 for Example 5 employing sulfonamide 14dd and carbonate 16a to afford 201 (80%) as a white solid. MS (ESI) 686.4 [(M+H)⁺]. ¹H NMR (CDCl₃, 400 MHz, rotamers) δ 7.70 (d, J=8.8 Hz, 2H), 7.47-7.28 (m, 5H), 7.13 (d, J=8.6 Hz, 2H), 6.98 (d, J=8.8 Hz, 2H), 6.89 (d, J=8.6 Hz, 2H), 5.73-5.62 (m, 1H), 5.03-4.87 (m, 1H), 4.04-3.76 (m, 3H), 3.75-3.64 (m, 2H), 3.20-3.05 (m, 1H), 3.05-2.81 (m, 3H), 2.81-2.70 (m, 1H), 1.70-1.43 (m, 2H).

### Example 38

Synthesis (3R,3aS,6aR)-hexahydrofuro[2,3-b]furan-3-yl-((2S,3R)-1-(4-(benzyloxy)phenyl)-3-hydroxy-4-(N-isobutyl-4-(trifluoromethoxy)phenylsulfonamido)butan-2-yl)carbamate-d14 (Compound 216).

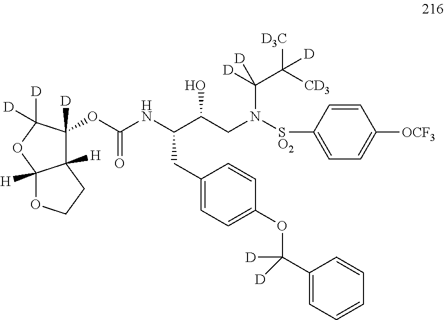

Step 1. tert-butyl-((2S,3R)-1-(4-(benzyloxy)phenyl)-3-hydroxy-4-(N-isobutyl-4-(trifluoro-methoxy)phenylsulfonamido)butan-2-yl)carbamate-d11 (14ee): Followed step 2 for Example 5 employing amino alcohol 12e and sulfonyl chloride 13c to afford sulfonamide 14ee (75%) as a white solid.

Step 2. (3R,3aS,6aR)-hexahydrofuro[2,3-b]furan-3-yl-((2S,3R)-1-(4-(benzyloxy)phenyl)-3-hydroxy-4-(N-isobutyl-4-(trifluoromethoxy)phenylsulfonamido)butan-2-yl)carbamate-d14 (216): Followed step 3 for Example 5 employing sulfonamide 14ee and carbonate 16a to afford 216 (64%) as a white solid. MS (ESI) 737.2 [(M+H)⁺]. ¹H NMR (CDCl₃, 400 MHz, rotamers) δ 7.83 (d, J=8.8 Hz, 2H), 7.46-7.29 (m, 7H), 7.12 (d, J=8.6 Hz, 2H), 6.89 (d, J=8.8 Hz, 2H), 5.78-5.61 (m, 1H), 5.05-4.88 (m, 1H), 4.08-3.74 (m, 3H), 3.74-3.64 (m, 1H), 3.55 (s, 1H), 3.23-3.08 (m, 1H), 3.07-2.94 (m, 2H), 2.94-2.79 (m, 1H), 2.79-2.68 (m, 1H), 2.36-2.25 (m, 0.3H), 1.95-1.80 (m, 0.3H), 1.74-1.60 (m, 0.7H), 1.58-1.48 (m, 0.7H).

### Example 39

Synthesis (3R,3aS,6aR)-hexahydrofuro[2,3-b]furan-3-yl-((2S,3R)-1-(4-(benzyloxy)phenyl)-3-hydroxy-4-(N-isobutyl-4-(difluoromethoxy)phenylsulfonamido)butan-2-yl)-carbamate-d14 (Compound 213).

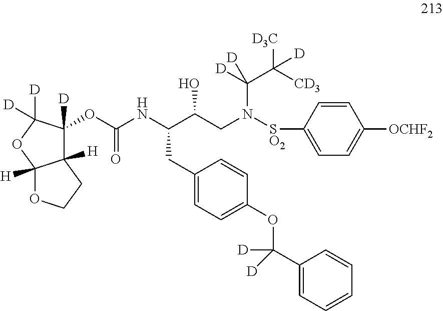

Step 1. tert-Butyl-((2S,3R)-1-(4-(benzyloxy)phenyl)-3-hydroxy-4-(N-isobutyl-4-(difluoro-methoxy)phenylsulfonamido)butan-2-yl)carbamate-d11 (14ff): Followed step 2 for Example 5 employing amino alcohol 11e and sulfonyl chloride 13d to afford sulfonamide 14ff (75%) as a white solid.

Step 2. (3R,3aS,6aR)-hexahydrofuro[2,3-b]furan-3-yl-((2S,3R)-1-(4-(benzyloxy)phenyl)-3-hydroxy-4-(N-isobutyl-4-(difluoromethoxy)phenylsulfonamido)butan-2-yl)-carbamate-d14 (213): Followed step 3 for Example 5 employing sulfonamide 14ff and carbonate 16a to afford 213 (75%) as a white solid. MS (ESI) 719.3 [(M+H)⁺]. ¹H NMR (CDCl₃, 400 MHz, rotamers) δ 7.78 (d, J=8.8 Hz, 2H), 7.47-7.29 (m, 5H), 7.24 (d, J=8.6 Hz, 2H), 7.12 (d, J=8.6 Hz, 2H), 6.89 (d, J=8.6 Hz, 2H), 6.59 (t, J=72.5 Hz, 1H), 5.78-5.61 (m, 1H), 5.05-4.90 (m, 1H), 4.05-3.75 (m, 3H), 3.75-3.63 (m, 1H), 3.57 (s, 1H), 3.21-3.06 (m, 1H), 3.05-2.67 (m, 4H), 2.36-2.25 (m, 0.4H), 1.95-1.80 (m, 0.4H), 1.74-1.60 (m, 0.6H), 1.58-1.48 (m, 0.6H).

### Example 40

Synthesis (3R,3aS,6aR)-hexahydrofuro[2,3-b]furan-3-yl-((2S,3R)-4-(2-amino-N-isobutylbenzo-[d]thiazole-6-sulfonamido)-3-hydroxy-1-phenylbutan-2-yl)carbamate-d12 (Compound 232) and general route to Compound 231 (Example 41). Compound 232 was prepared as shown in Scheme 24 and as described below.

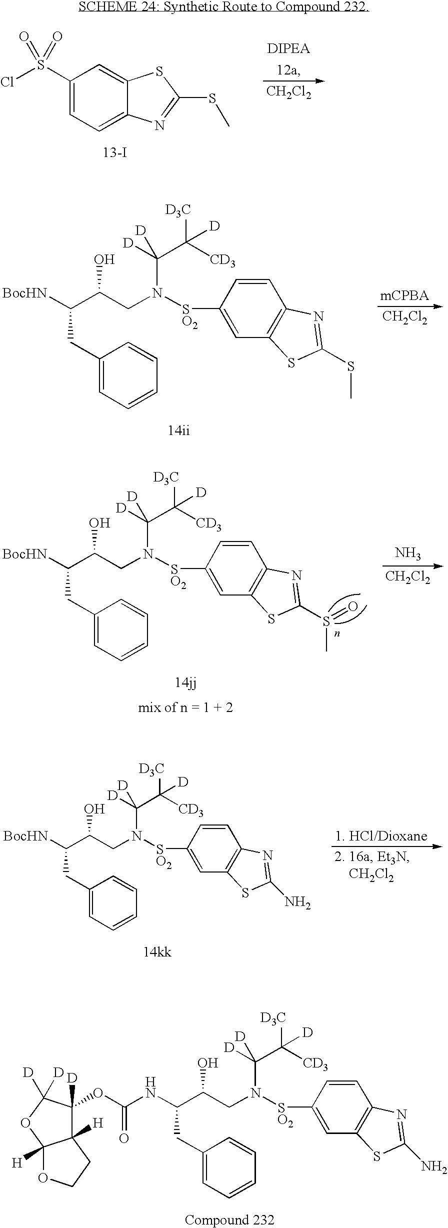

Step 1. tert-Butyl-((2S,3R)-3-hydroxy-4-(N-isobutyl-2-(methylthio)benzo[d]thiazole-6-sulfonamido)-1-phenylbutan-2-yl)carbamate-d9 (141i): Followed step 2 for 220 employing amino alcohol 12a and sulfonyl chloride 13-1 to afford sulfonamide 14ii (96%). MS (ESI) 489.2 [(M-Boc+H)⁺].

Step 2. tert-Butyl-((2S,3R)-3-hydroxy-4-(N-isobutyl-2-(methylsulfonyl)benzo[d]thiazole-6-sulfonamido)-1-phenylbutan-2-yl)carbamate-d9 (14jj): To a solution of 14ii (172 mg, 0.292 mmol) in CH₂Cl₂ (4.00 mL) was added mCPBA (70%, 108 mg, 0.438 mmol). The reaction stirred at room temperature for 2 hours then was diluted with CH₂Cl₂ and washed sequentially with 10% Na₂S₂O₃, sat NaHCO₃, and brine. The organic layer was dried (Na₂SO₄), filtered through Celite® and concentrated under reduced pressure to afford 14jj (177 mg, 99%) as a white solid and as a mixture of sulfone and sulfoxide oxidation states. MS (ESI) sulfone: 521.2 [(M-Boc+H)⁺]sulfoxide: 505.3 [(M-Boc+H)⁺].

Step 3. tert-Butyl-((2S,3R)-4-(2-amino-N-isobutylbenzo[d]thiazole-6-sulfonamido)-3-hydroxy-1-phenylbutan-2-yl)carbamate-d9 (14kk): To a solution of 14jj (255 mg, 0.416 mmol) in CH₂Cl₂ (1 mL) was added ammonia (1.04 mL, 2.08 mmol, 2M in iPrOH). The reaction stirred at room temperature for 2.5 hours then another 1 mL ammonia (2 M in iPrOH) was added. The reaction was stirred at 40° C. for 15 hours then cooled to room temperature and concentrated under reduced pressure. The resulting residue was purified by silica gel column chromatography on an ISCO system (0-60% EtOAc/heptane) to afford 14kk (200 mg, 86%) as a white solid. MS (ESI) 458.3 [(M-Boc+H)⁺].

Step 4. (3R,3aS,6aR)-hexahydrofuro[2,3-b]furan-3-yl-((2S,3R)-4-(2-amino-N-isobutylbenzo-[d]thiazole-6-sulfonamido)-3-hydroxy-1-phenylbutan-2-yl)carbamate-d12 (232): Followed step 3 for Example 5 employing sulfonamide 14kk and carbonate 16a to afford 232 (73%) as a white solid. MS (ESI) 617.3 [(M+H)⁺]. ¹H NMR (CDCl₃, 400 MHz, rotamers) δ 8.03 (s, 1H), 7.69 (d, J=8.6 Hz, 1H), 7.59 (d, J=8.6 Hz, 1H), 7.34-7.15 (m, 5H), 5.69-5.62 (m, 1H), 5.59 (s, 2H), 5.08-4.93 (m, 1H), 3.97-3.76 (m, 4H), 3.74-3.62 (m, 2H), 3.24-2.73 (m, 5H), 1.69-1.55 (m, 1H), 1.52-1.41 (m, 1H).

### Example 41

Synthesis (3R,3aS,6aR)-hexahydrofuro[2,3-b]furan-3-yl-((2S,3R)-3-hydroxy-4-(N-isobutyl-2-(methylamino)benzo[d]thiazole-6-sulfonamido)-1-phenylbutan-2-yl)carbamate-d12 (Compound 231). Compound 231 was prepared as shown generally in Scheme 24 and as described below.

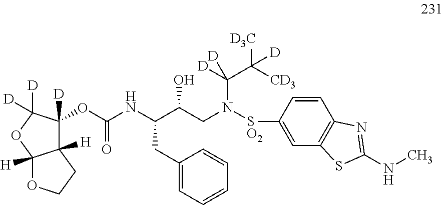

Step 1. tert-butyl-((2S,3R)-3-hydroxy-4-(N-isobutyl-2-(methylamino)benzo[d]thiazole-6-sulfonamido)-1-phenylbutan-2-yl)carbamate-d9 (14-ll): Followed procedure for 14kk employing methylamine in THF in place of ammonia in iPrOH to afford 14-ll (78%). MS (ESI) 472.3 [(M-Boc+H)⁺].

Step 2. (3R,3aS,6aR)-hexahydrofuro[2,3-b]furan-3-yl-((2S,3R)-3-hydroxy-4-(N-isobutyl-2-(methylamino)benzo[d]thiazole-6-sulfonamido)-1-phenylbutan-2-yl)carbamate-d12 (231): Followed step 3 for Example 5 employing sulfonamide 14-11 and carbonate 16a to afford 231 (81%) as a white solid. MS (ESI) 631.4 [(M+H)⁺]. ¹H NMR (CDCl₃, 400 MHz, rotamers) δ 8.01 (s, 1H), 7.67 (d, J=8.6 Hz, 1H), 7.57 (d, J=8.6 Hz, 1H), 7.34-7.15 (m, 5H), 5.71-5.57 (m, 2H), 5.08-4.93 (m, 1H), 3.97-3.76 (m, 3H), 3.74-3.62 (m, 2H), 3.24-2.73 (m, 5H), 3.16 (s, 3H), 1.69-1.55 (m, 1H), 1.52-1.41 (m, 1H).

### Example 42

Synthesis 45-(N-((2R,3S)-3-(((((3R,3aS,6aR)-hexahydrofuro[2,3-b]furan-3-yl)oxy)carbonyl)amino)-2-hydroxy-4-phenylbutyl)-N-isobutylsulfamoyl)benzofuran-3-yl)methyl)carbamic acid ethyl ester-d12 (Compound 235). Compound 235 was prepared as shown in Scheme 25 and as described below.

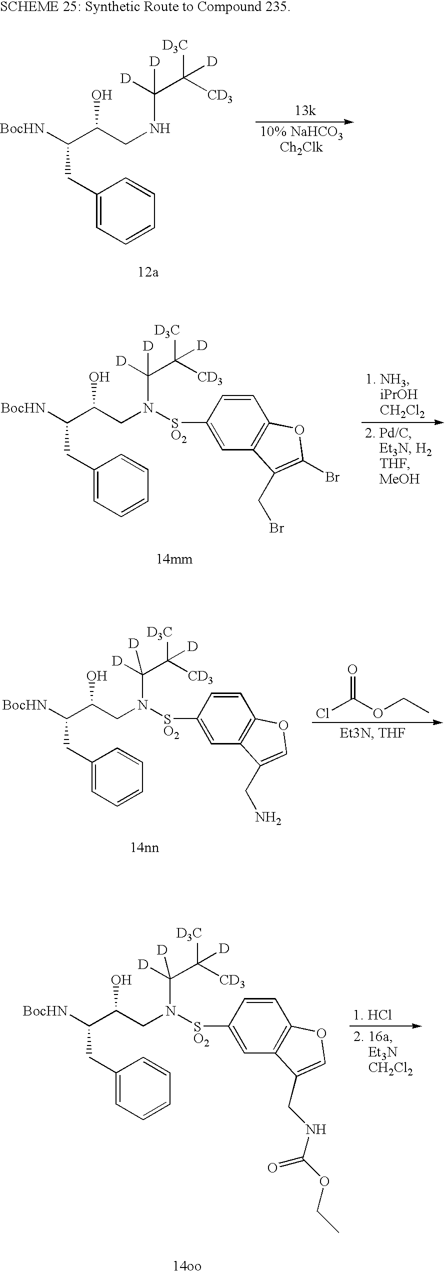

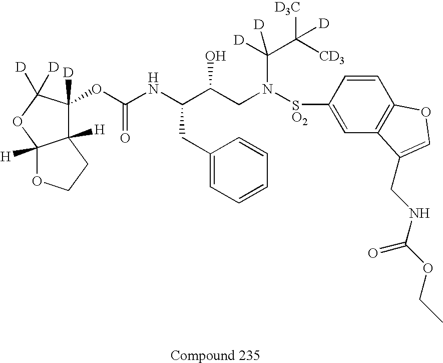

Step 1. tert-Butyl-((2S,3R)-4-(2-bromo -3-(bromomethyl)-N-isobutylbenzofuran-5-sulfon-amido)-3-hydroxy-1-phenylbutan-2-yl)carbamate-d9 (14 mm): Followed step 2 for Example 5 employing 10% NaHCO₃ as base in place of N,N-diisopropylethylamine, amino alcohol 12a and sulfonyl chloride 13k to afford sulfonamide 5mm (99%).

Step 2. tert-Butyl-((2S,3R)-4-(3-(aminomethyl)-N-isobutylbenzofuran-5-sulfonamido)-3-hydroxy-1-phenylbutan-2-yl)carbamate-d9 (14nn): To a solution of 14mm (263 mg, 0.377 mmol) in CH₂Cl₂ (10 mL) at 0° C. was added 10 mL of a 2M solution of ammonia in 2-propanol. The reaction stirred at room temperature for 24 hours then was concentrated. The resulting white solid was diluted with THF and methanol was added until dissolution was achieved. Triethylamine (105 μL, 0.754 mmol) was then added and the vessel was evacuated several times with nitrogen. 10% Pd/C (50% wet, 20 mg) was then added and the vessel was again purged with nitrogen several times. The reaction then stirred under an atmosphere of H₂ for 2 hours. LCMS indicated the sole presence of starting material therefore an additional 20 mg Pd/C was added and the reaction continued to stir under an atmosphere of H₂ for 1 hour. The reaction was deemed complete by LCMS and the contents were filtered through Celite®. The filter cake was rinsed with methanol and the filtrate was concentrated under reduced pressure. The resulting residue was purified by silica gel column chromatography on in ISCO system (0-5% MeOH/CH₂Cl₂) to afford 14nn (82 mg, 39%) as a white foam. MS (ESI) 555.4 [(M+H)⁺].

Step 3. ((5-(N-((2R,3S)-3-((tert-butoxycarbonyl)amino)-2-hydroxy-4-phenylbutyl-N-isobutylsulfamoyl)benzofuran-3-yl)methyl)carbamic acid ethyl ester-d9 (14oo): To a solution of 14nn (82 mg, 0.148 mmol) in THF (2.50 mL) was added triethylamine (31 μL, 0.222 mmol) followed by ethyl chloroformate (18 μL, 0.192 mmol). The reaction stirred at room temperature for 1 hour then was diluted with CH₂Cl₂ (10 mL) and washed with sat. NaHCO₃ (3×10 mL). The organic layer was then dried (Na₂SO₄), filtered through Celite® and concentrated under reduced pressure to afford 1400 (86 mg, 93%). MS (ESI) 527.2 [(M−Boc+H)⁺].

Step 4. ((5-(N-((2R,3S)-3-(((((3R,3aS,6aR)-hexahydrofuro[2,3-b]furan-3-yl)oxy)carbonyl)amino)-2-hydroxy-4-phenylbutyl)-isobutylsulfamoyl)benzofuran-3-yl)methyl)carbamic acid ethyl ester-d12 (235): Followed step 3 for Example 5 employing sulfonamide 1400 and carbonate 16a to afford 235 (50%) as a white solid. MS (ESI) 686.3 [(M+H)⁺]. ¹H NMR (CDCl₃, 400 MHz, rotamers) δ 8.14 (s, 1H), 7.80-7.64 (m, 2H), 7.59 (d, J=8.6 Hz, 1H), 7.32-7.13 (m, 5H), 5.68-5.55 (m, 1H), 5.46 (d, J=9.6 Hz, 1H), 5.22 (br s, 1H), 4.61 (dd, J=8.1, 15.9 Hz, 1H), 4.43 (dd, J=4.8, 14.9 Hz, 1H), 4.22-4.08 (m, 2H), 4.03 (br s, 1H), 3.95-3.77 (m, 2H), 3.76-3.59 (m, 1H), 3.50 (s, 1H), 3.32-2.99 (m, 3H), 2.97-2.68 (m, 2H), 1.70-1.48 (m, 1H), 1.46-1.35 (m, 1H), 1.25 (t, J=8.3 Hz, 3H).

### Example 43

### Determination of Metabolic Stability of Test Compounds in

Human Liver Microsomes. Human liver microsomes (20 mg/mL) were obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) were purchased from Sigma-Aldrich.

7.5 mM stock solutions of test compounds were prepared in DMSO. The 7.5 mM stock solutions were diluted to 12.5 μM in acetonitrile (ACN). The 20 mg/mL human liver microsomes were diluted to 0.625 mg/mL in 0.1 M potassium phosphate buffer, pH 7.4, containing 3 mM MgCl₂. The diluted microsomes (375 μL) were added to wells of a 96-well deep-well polypropylene plate in triplicate. 10 to 40 μL of the 12.5 μM test compound was added to the microsomes and the mixture was pre-warmed for 10 minutes. Reactions were initiated by addition of 125 μL of pre-warmed NADPH solution. The final reaction volume was 0.5 mL and contained 0.5 mg/mL human liver microsomes, 0.25-1.0 μM test compound, and 2 mM NADPH in 0.1 M potassium phosphate buffer, pH 7.4, and 3 mM MgCl₂. The reaction mixtures were incubated at 37° C., and 50 μL aliquots were removed at 0, 5, 10, 20, and 30 minutes and added to shallow-well 96-well plates which contained 50 μL of ice-cold ACN with internal standard to stop the reactions. The plates were stored at 4° C. for 20 minutes after which 100 μL of water was added to the wells of the plate before centrifugation to pellet precipitated proteins. Supernatants were transferred to another 96-well plate and analyzed for amounts of parent compound remaining by LC-MS/MS using an Applied Bio-systems API 4000 mass spectrometer. 7-ethoxycoumarin (1 μM) was used as the positive control substrate.

Data analysis: The in vitro half-lives (tᵢᵢ₂s) for test compounds were calculated from the slopes of the linear regression of % parent remaining (ln) vs incubation time relationship using the following formula:

in vitro t_{1/2}=0.693/k, where k=−[slope of linear regression of % parent remaining(ln) vs incubation time]

Data analysis was performed using Microsoft Excel Software.

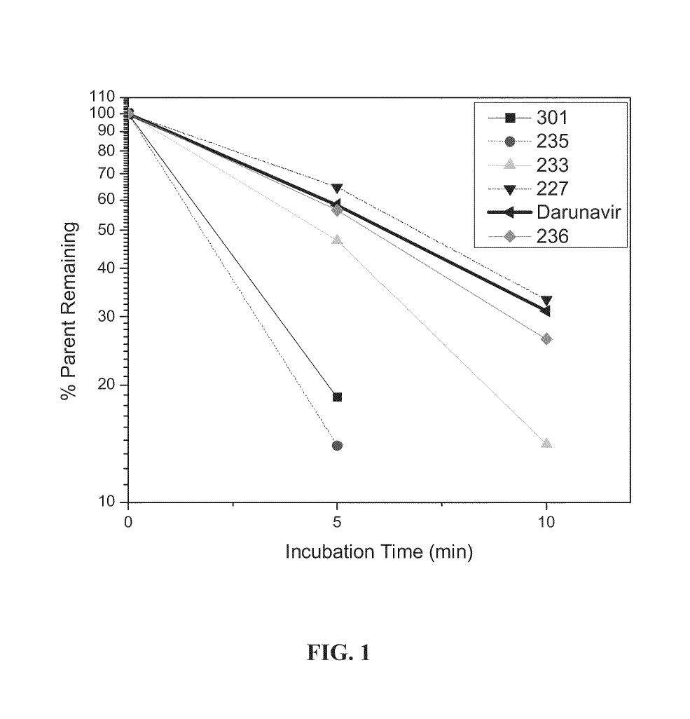

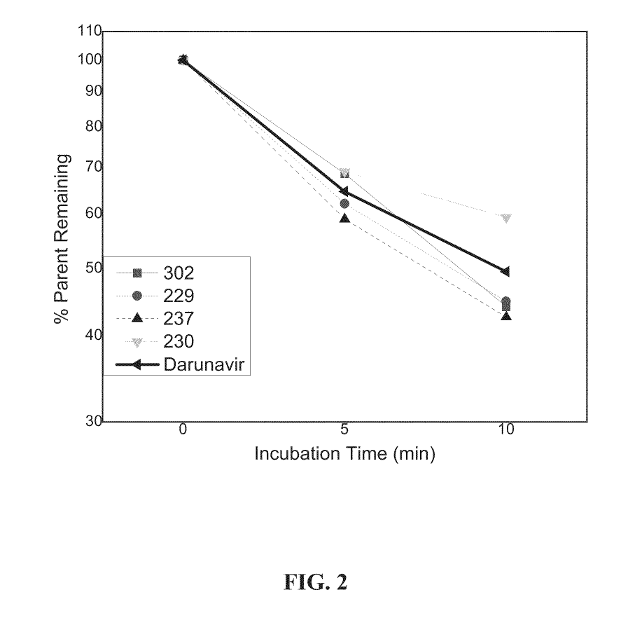

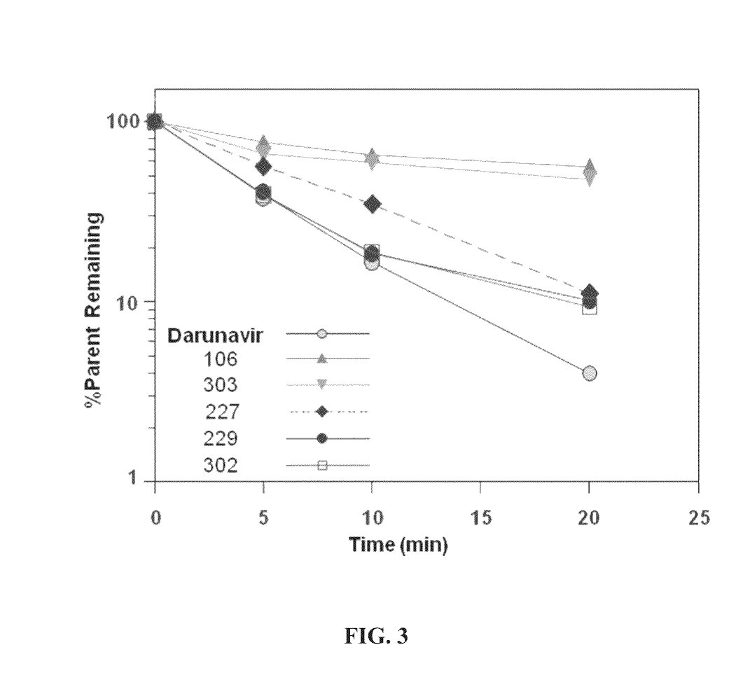

FIG. 1, FIG. 2, FIG. 3, Table 2, Table 3 and Table 4 show the results of this experiment.

TABLE 2
Experimental values of, and calculated average values of, Half-life
(T_{1/2}) in Human Liver Microsomes for deuterated compounds
233, 235, 227 and 236; and non-dueterated compounds 301 (the
non-deuterated counterpart of 235) and darunavir.

T_{1/2} (min)

| Cmpd Tested | Exp A | Exp B | Average | SD |
|---|---|---|---|---|
| Darunavir | 6.4 | 5.4 | 5.9 | 0.3 |
| 301 | 1.9 | 1.5 | 1.7 | 0.3 |
| 235 | 2.4 | 1.5 | 2.0 | 0.6 |
| 233 | 4.7 | 2.4 | 3.5 | 1.7 |
| 227 | 9.0 | 4.3 | 6.6 | 3.3 |
| 236 | 7.1 | 3.6 | 5.4 | 2.4 |

In the structure of 301 (shown below) all atoms are at their natural isotopic abundance:

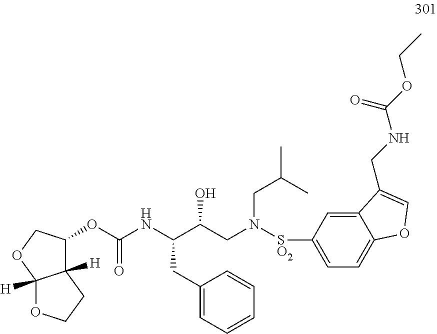

TABLE 3
Experimental values of, and calculated average values of, Half-life
in Human Liver Microsomes for deuterated compounds 229, 237,
and 230; and non-deuterated compounds 302 (the non-deuterated
counterpart of compounds 229, 237, and 230) and darunavir.

T_{1/2} (min)

| Cmpd Tested | Exp C | Exp D | Average | SD |
|---|---|---|---|---|
| Darunavir | 9.7 | 10.0 | 10.0 | 0.3 |
| 302 | 8.2 | 8.7 | 8.5 | 0.3 |
| 229 | 8.7 | 8.6 | 8.6 | 0.1 |
| 237 | 8.3 | 7.9 | 8.1 | 0.3 |
| 230 | 11.7 | 15.1 | 13.4 | 2.4 |

In the structure of 302 (shown below) all atoms are at their natural isotopic abundance:

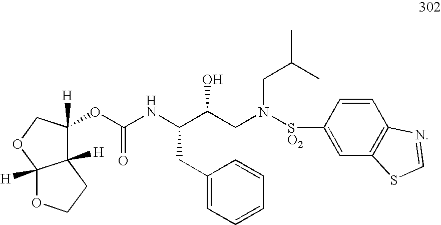

Compound 302 is the non-deuterated counterpart of compounds 230, 229 and 237.

Compound 230 shows an increase in stability over darunavir of 36% (average of two runs), and an even greater increase in stability over 302, its non-deuterated counterpart.

TABLE 4
Experimental values of, and calculated average values of, Half-life in
Human Liver Microsomes for deuterated compounds 106, 227,
and 229; and non-dueterated compounds 303 (the non-deuterated
counterpart of 106), 302, darunavir and brecanavir.

| | T_{1/2 (min)} | | | | | | |
|---|---|---|---|---|---|---|---|
| Cmpd | | Expt | Expt | Expt | Expt | Expt | |
| Tested | Expt E | F | G | H | I | J | Average ± SD |
| Darunavir | 4.4 | 4.3 | 4.2 | 3.9 | 4.5 | 4.5 | 4.3 ± 0.2 |
| 106 | 31.2 | 21.2 | | 18.1 | 21.5 | 46.7 | 23.0 ± 5.7 |
| 303 | | | | | 21.1 | 19.9 | 20.5 |
| 227 | | | 5.7 | 5.3 | 7.2 | 7.1 | 6.3 ± 1.0 |
| 229 | | | 6.4 | 6.7 | 6.0 | 5.8 | 6.2 ± 0.4 |
| 302 | | | | | 5.6 | 6.4 | 6.0 |
| Brecanavir | | | | | 2.0 | 2.0 | 2.0 |

In the structure of 303 (shown below) all atoms are at their natural isotopic abundance:

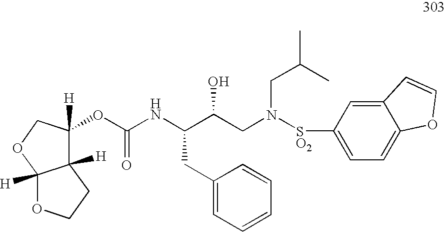

Compound 106 shows a very significant increase in stability over darunavir and brecanavir.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of Formula I: 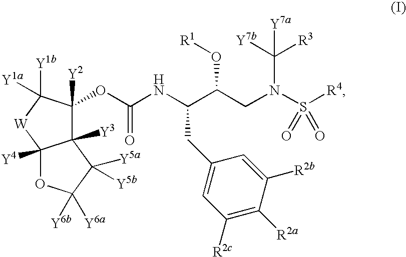
or a pharmaceutically acceptable salt thereof, wherein:
W is —O—, —CH₂—, or —CD₂—;
each Y is independently selected from hydrogen or deuterium;
R¹ is hydrogen or —(C₁-C₁₁ alkylene)-R⁶, wherein, the C₁-C₁₁ alkylene is optionally substituted by one or more groups independently selected from halo, cyano, —OH, ═O, —SH, —PO₃H, —PO₃(C₁₋₆ alkyl), ═N, —NH₂, NH(C₁-C₄ alkyl), N(C₁-C₄ alkyl)₂, —C₁-C₄ alkyl, cycloheteroalkyl, cycloheteroalkylalkyl, aryl, arylalkyl, heteroaryl, heteroarylalkyl, or a side group of a naturally occurring amino acid, and up to 4 methylene units in the C₁-C₁₁ alkylene are optionally and independently replaced with —O—, —S—, —S(O)—, S(O)₂—, —P(O)₂—, —P(O)(OH)—, —NH—, and —N(C₁-C₆ alkyl)-, provided that the terminal end of R¹ bonded to the oxygen is not oxygen or nitrogen, wherein:
R⁶ is selected from hydrogen, —N(R⁷)(R⁷), optionally substituted C₁-C₈ alkyl, C₁-C₈ alkoxy, a heteroaryl or cycloheteroalkyl wherein the heteroaryl or cycloheteroalkyl is optionally substituted with C₁-C₈ alkyl, and wherein each R⁷ is independently selected from hydrogen, C₁-C₈ alkyl, and C₁-C₈ alkoxy;
R^{2a} is hydrogen, —OH, —F, —O—Z—R¹⁰, or C₁₋₄ alkyl optionally substituted with one or more —F, wherein
Z is a C₁₋₄ alkylene that is optionally substituted with deuterium; and
R¹⁰ is selected from hydrogen, deuterium, phenyl, cyanophenyl, pyridyl, 3-cyanopyridyl, pyrazinyl, pyrazol-1-yl, 3,5-dimethylpyrazol-1-yl, isoxazol-3-yl, 5-methyl-isoxazol-3-yl, 2-methyl-thiazol-4-yl, 5-methyl-thiazol-4-yl, 2,5-dimethyl-thiazol-4-yl, 2-methyl-thiazol-5-yl, 4-methyl-thiazol-5-yl, 2,4-dimethyl-thiazol-5-yl, 2-thienyl, 4-morpholinyl, 4-methylpiperazin-1-yl, (C₁₋₄ alkyl)aminocarbonyl, trifluoromethyl, hydroxymethyl, (C₁ alkyl)sulfonylamino, (C₁₋₄ alkoxy)methylcarbonylamino, (C₁₋₄ alkyl)carbonylamino, phenylcarbonylamino, (C₁₋₄ alkoxy)carbonylamino, 2-furanylcarbonylamino, 2-thienylcarbonylamino, (C₁₋₄ alkyl)carbamyloxy, bis(methoxyethyl)amino, C₆H₅C(═N—CN)—NH—, 1H-pyrazol-5-ylamino, pyrimidin-2-ylamino, 1H-1,2,4-triazol-1-yl, thiazol-2-yl, thiazol-4-yl, thiazol-5-yl, and 5-phenyl-1,2,4-oxadiazol-3-yl, wherein any methyl, alkyl or alkoxy moiety in R¹⁰ is optionally substituted with one or more deuterium;
each of R^{2b} and R^{2c} is independently hydrogen or —F;
R³ is a group that is optionally substituted with one or more deuterium and is selected from phenyl, C₃-C₆ cycloalkyl, C₅-C₆ cycloalkenyl, —CH₂—CH(CH₃)₂, —CH(CH₃)₂, —CH(C₂H₅)₂, —CH(CH₃)—C₂H₅, —CH═CH₂, and —C(CH₃)₂—(CH₂)ₘ—NH—R¹¹, wherein:
R¹¹ is selected from hydrogen, —C(O)OCH₃, —C(O)CH₃, —C(O)NHCH₃ and —S(O)₂CH₃; and
m is 1, 2, 3, 4 or 5; and
R⁴ is a group that is optionally substituted with one or more deuterium and is selected from 2,3-dihydrobenzofuran-5-yl, 3-oxo-2,3-dihydrobenzofuran-5-yl, chromanyl-6-yl, 4-oxo-chromanyl-6-yl, 4-oxo-4H-chromenyl-6-yl, 2,3-dihydrobenzo[b][1,4]dioxin-6-yl, 3,4-dihydro-2H-benzo[b][1,4]oxazin-6-yl, 3,4-dihydro-2H-benzo[b][1,4]oxazin-7-yl, 3-oxo-3,4-dihydro-2H-benzo[b][1,4]oxazin-7-yl, 3-oxo-3,4-dihydro-2H-benzo[b][1,4]oxazin-6-yl, 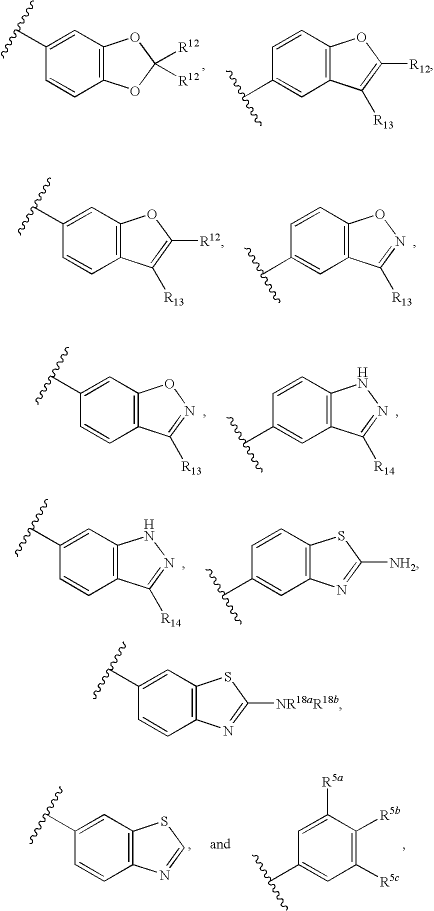
wherein:
each R⁵ is independently selected from hydrogen, —F, —NH₂, —NHC(O)C₁₋₆alkyl optionally substituted with one or more deuterium, such as —NHC(O)CH₃ or —NHC(O)CD₃, —NHC(O)C₃₋₁₀ cycloalkyl optionally substituted with one or more deuterium, —OH, —OCH₃, —OCD₃, —OCF₃, —OCHF₂, —CH₃, —CD₃, —CF₃, —CN, —CH₂—OCD₃, —CD₂-OCD₃, —CD₂-OCH₃, —CH₂OH, —CD₂OH, —OCH₂CH₃, —OCD₂CD₃, —OCH(CH₃)₂, —OCD(CH₃)₂, —OCD(CD₃)₂, and —OCH(CD₃)₂;
each R¹² is independently selected from hydrogen, deuterium, —CH₃, and —CD₃;
R¹³ is selected from hydrogen, deuterium, —CH₃, —CD₃, —CH₂—O—CH₃, —CH₂—O-CD₃, —CD₂-O-CD₃, —CH₂-(1-piperidinyl), —CH₂-(4-morpholinyl), —(CH₂)_{w}-Q-C(O)-Q-R¹⁵, —(CD₂)_{w}-Q-C(O)-Q-R¹⁵, —CH₂—NH—X—R¹⁵, and —CD₂-NH—X—R¹⁵, wherein:
X is selected from a bond, —C(O)—, —CO₂— and —SO₂—;
R¹⁵ is selected from C₁₋₆ alkyl, C₃₋₆ cycloalkyl, or C₄₋₁₀ (cycloalkyl)alkyl, wherein R¹⁵ is optionally substituted with one or more substituents independently selected from deuterium, —CF₃, phenyl, —CH₂-phenyl, —CD₂-phenyl, 2-furanyl, —CH₂-(2-furanyl), —CD₂-(2-furanyl), —CH₂-(2-benzofuranyl), —CD₂-(2-benzofuranyl), 2-pyridyl, 3-pyridyl, —CH₂-(2-pyridyl), —CD₂-(2-pyridyl), —CH₂-(3-pyridyl), —CD₂-(3-pyridyl), 4-thiazolyl, —CH₂-(4-thiazolyl), and —CD₂-(4-thiazolyl);
w is 1 or 2; and
each Q is independently NH, O, CH₂ or CD₂;
R¹⁴ is selected from hydrogen, deuterium, —CH₃, —CD₃, and —N(R¹⁶)₂;
each R¹⁶ is independently selected from C₁₋₆ alkyl, C₃₋₆ cycloalkyl, and C₄₋₁₀ (cycloalkyl)alkyl, wherein R¹⁶ is optionally substituted with one or more substituents independently selected from deuterium, halo, hydroxyl, cyano, —N(R¹⁷)₂, —C(O)—R¹⁷, —CO₂R¹⁷, —C(O)—N(R¹⁷)₂;
each R¹⁷ is independently selected from C₁₋₆ alkyl, C₃₋₆ cycloalkyl, or C₄₋₁₀ (cycloalkyl)alkyl, wherein R¹⁷ is optionally substituted with one or more deuterium:
R^{18a} is selected from hydrogen, deuterium, —CH₃, —CD₃, 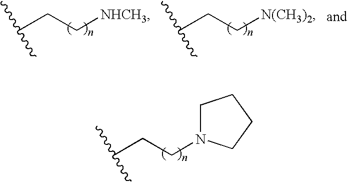
wherein n is 1 or 2, and wherein each of 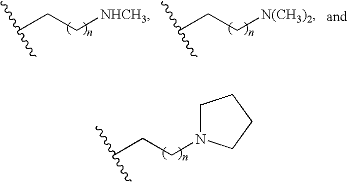
is optionally substituted on one or more carbon atoms with deuterium; and
R^{18b} is selected from hydrogen; deuterium; C₁₋₆ alkyl optionally substituted with one or more of halo, aryl, or heteroaryl; cyano; —COOH; —OC₁₋₆ alkyl; —NH₂; —NH(C₁₋₆ alkyl); N(C₁₋₆alkyl)₂; a 3- to 10-membered cycloheteroalkyl; C₆₋₁₀ aryl, a 5- to 10-membered heteroaryl; —C(O)OC₁₋₆ alkyl; —C(O)NHC₁₋₆ alkyl; 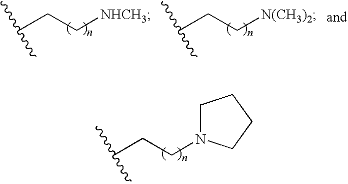
wherein n is 1 or 2, and wherein each of C₁₋₆ alkyl, —OC₁₋₆ alkyl, —NHC₁₋₆ alkyl, N(C₁₋₆alkyl)₂, —C(O)OC₁₋₆ alkyl, —C(O)NHC₁₋₆ 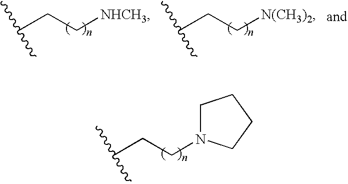
is optionally substituted on one or more carbon atoms with deuterium;
provided that when W is —O—, R^{2a} is hydrogen and R³ is isopropyl optionally substituted with one or more deuterium, then R⁴ is other than 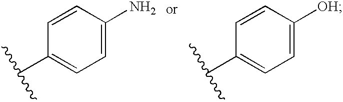
and further provided that at least one Y is deuterium or at least one of R¹, R^{2a}, R³ or R⁴ comprises a deuterium atom.

2. The compound of claim 1, wherein R¹ is hydrogen.

3. The compound of claim 1, wherein:
Y^{1a} and Y^{1b} are the same;
Y^{5a} and Y^{5b} are the same;
Y^{6a} and R^{6b} are the same;
Y^{7a} and Y^{7b} are the same; and
R³ is selected from —CH(CH₃)₂, —CD(CH₃)₂, and —CD(CD₃)₂.

4. The compound of claim 1, wherein W is —O—.

5. The compound of claim 4, wherein:
each Y¹, Y², Y³, Y⁴, each Y⁵ and each Y⁶ is deuterium; and
R³ is selected from —CH(CH₃)₂ and —CD(CD₃)₂.

6. The compound of claim 4, wherein:
each Y¹ and Y² is deuterium;
Y³, Y⁴, each Y⁵ and each Y⁶ is hydrogen; and
R³ is selected from —CH(CH₃)₂, —CD(CH₃)₂, and —CD(CD₃)₂.

7. The compound of claim 5, wherein R³ is —CD(CD₃)₂.

8. The compound of claim 1, wherein:
R^{2a} is H or —O—Z—R¹⁰; and
R^{2b} and R^{2c} are hydrogen.

9. The compound of claim 8, wherein R^{2a} is —O—Z—R¹⁰; and
R¹⁰ is selected from pyridyl, pyrazinyl, pyrazol-1-yl, 3,5-dimethylpyrazol-1-yl, isoxazol-3-yl, 5-methyl-isoxazol-3-yl, 2-methyl-thiazol-4-yl, 5-methyl-thiazol-4-yl, 2,5-dimethyl-thiazol-4-yl, 2-methyl-thiazol-5-yl, 4-methyl-thiazol-5-yl, 2,4-dimethyl-thiazol-5-yl, 4-morpholinyl, 4-piperazinyl, methylsulfonylamino, methoxymethylcarbonylamino, methylcarbamyloxy, and (C₁-C₄ alkoxy)carbonylamino, wherein any methyl or alkoxy moiety in R¹⁰ is optionally substituted with one or more deuterium.

10. The compound of claim 9, wherein R¹⁰ is selected from

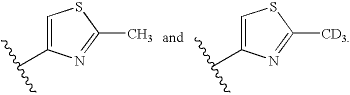

11. The compound of claim 1, wherein R⁴ is selected from: 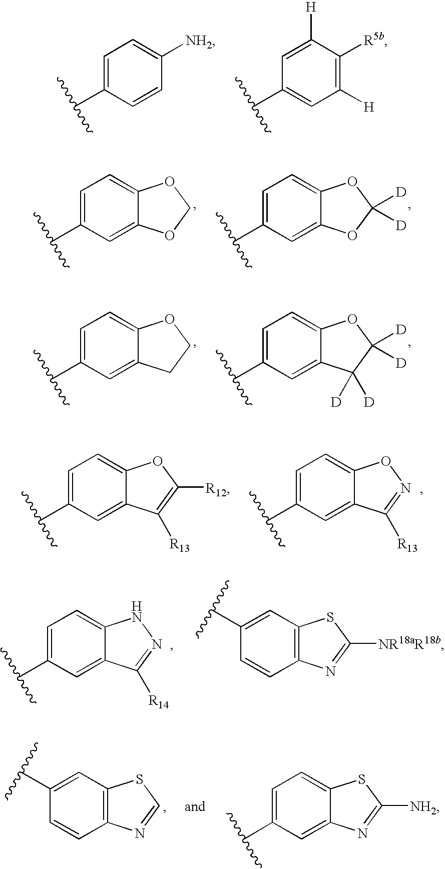
wherein R^{5b} is —NHC(O)C₁₋₆alkyl optionally substituted with one or more deuterium or —NHC(O)C₃₋₁₀cycloalkyl optionally substituted with one or more deuterium.

12. The compound of claim 11, wherein R⁴ is selected from any one of the following:

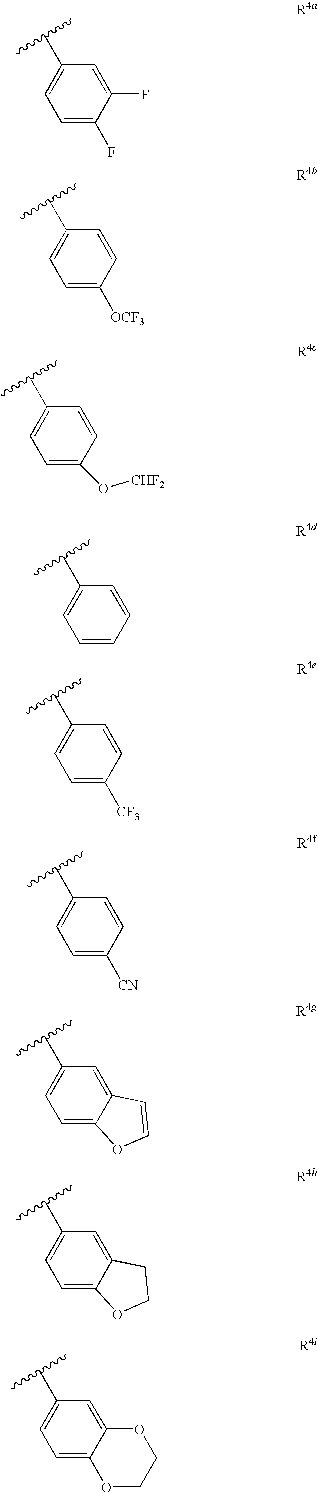

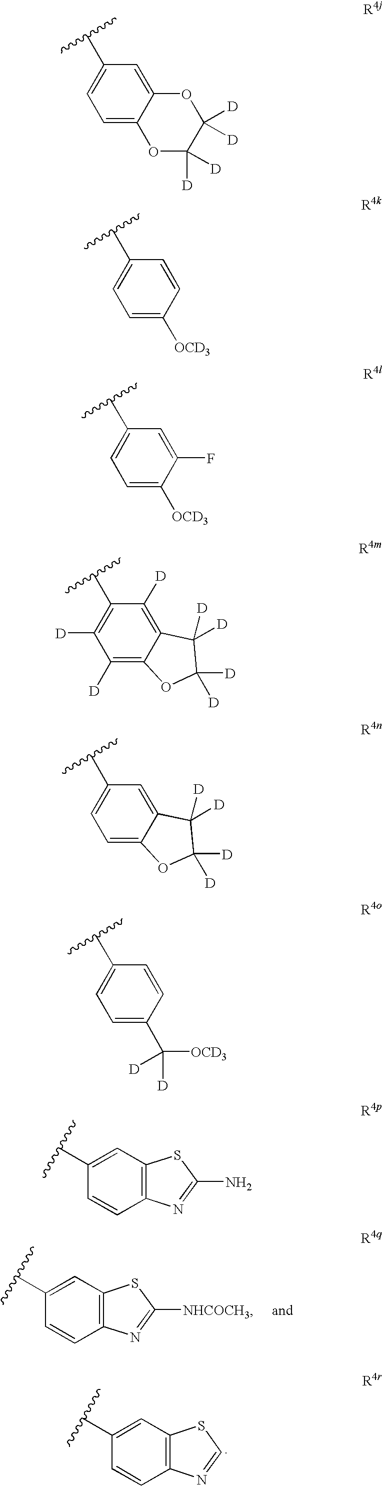

13. The compound of claim 1, selected from any one of the following compounds or a pharmaceutically acceptable salt thereof:

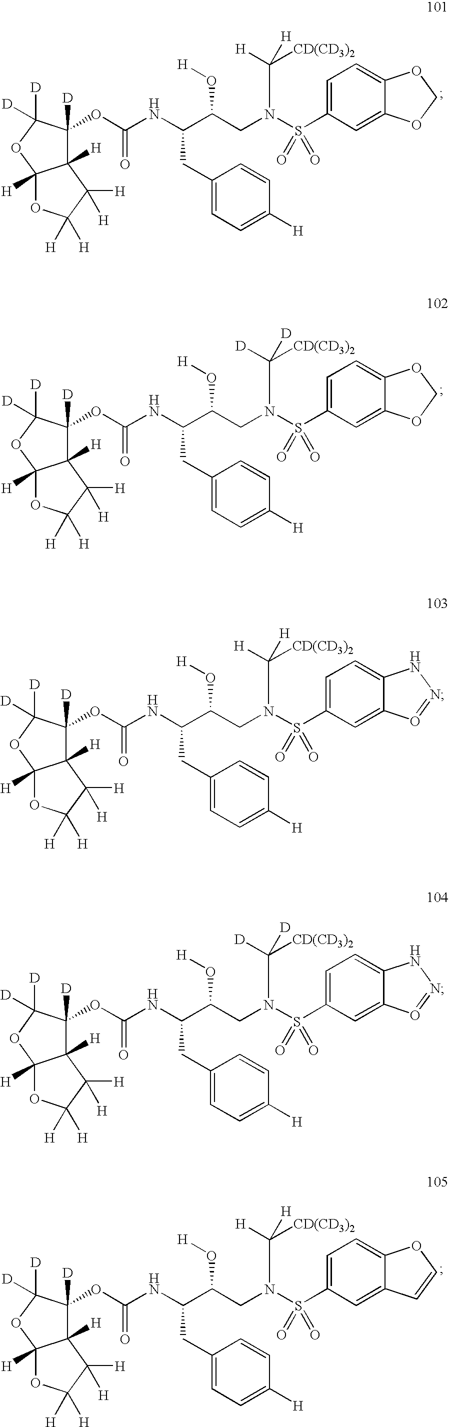

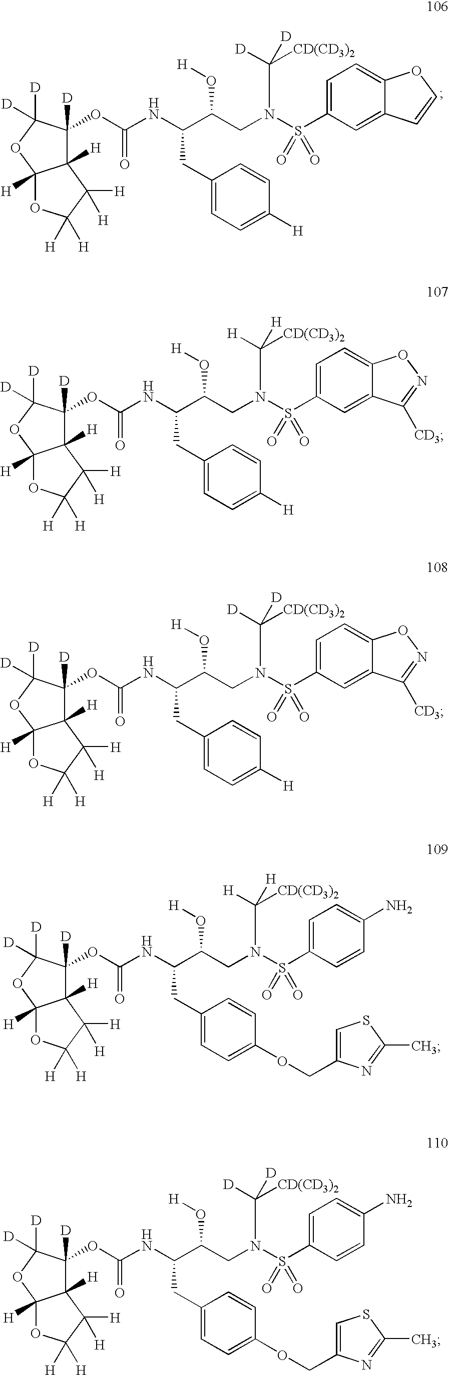

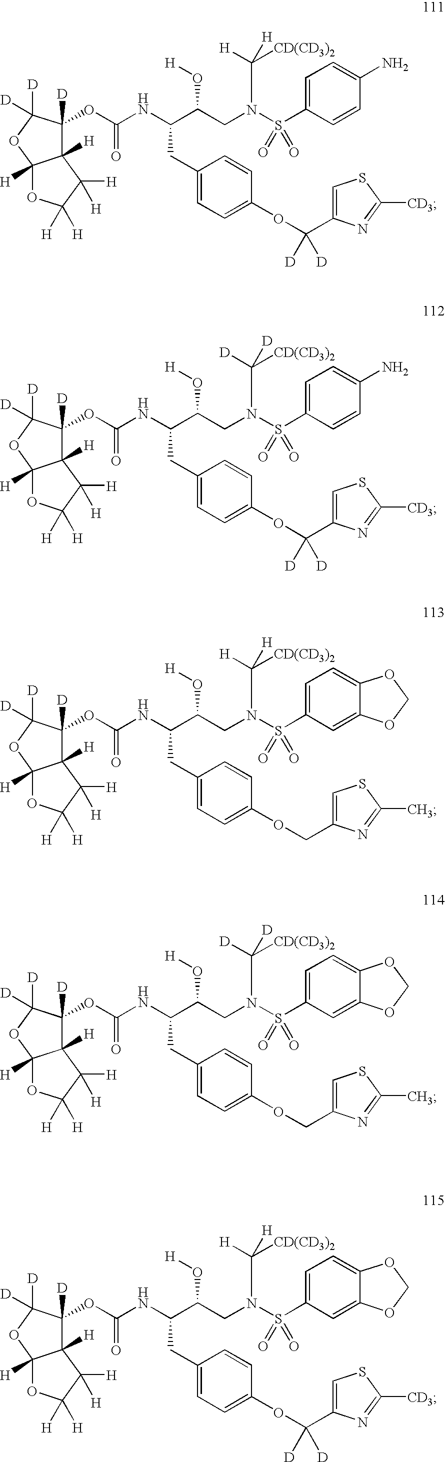

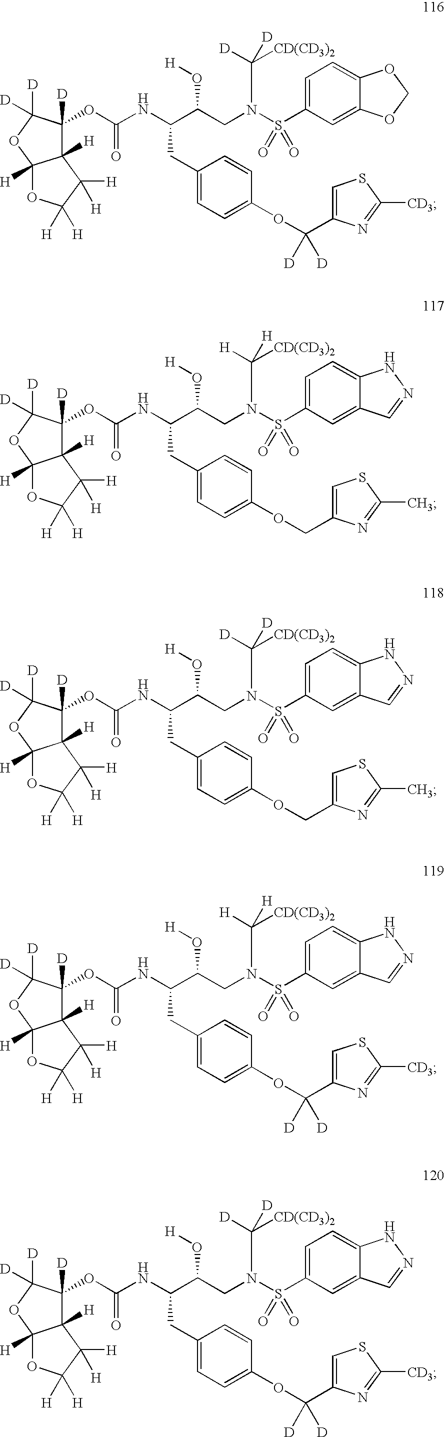

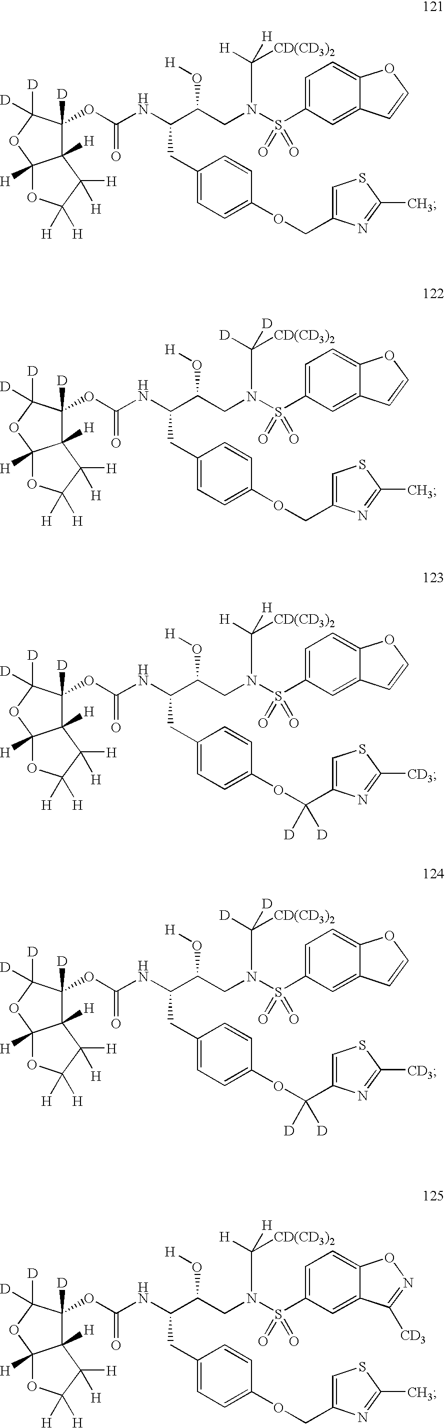

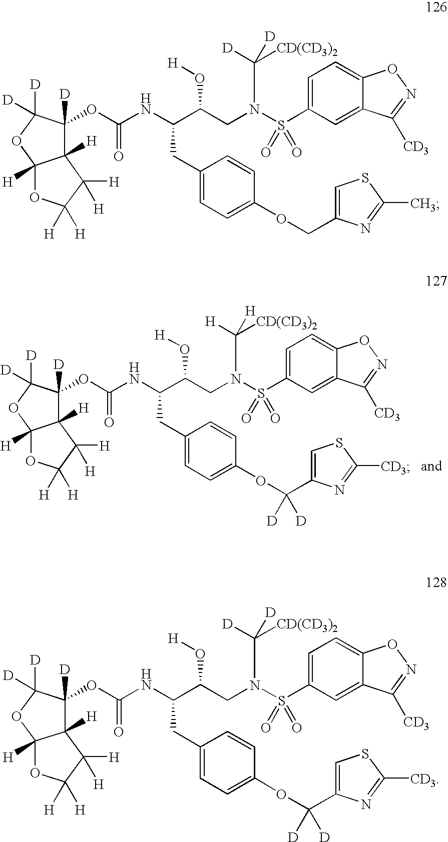

14. The compound of claim 1, selected from any one of the following compounds or a pharmaceutically acceptable salt thereof:

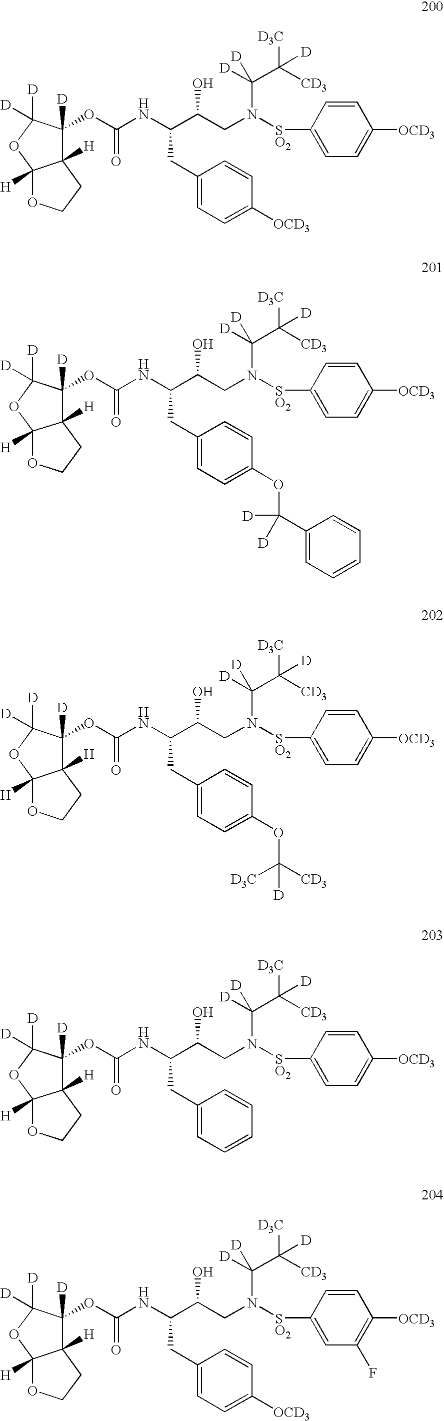

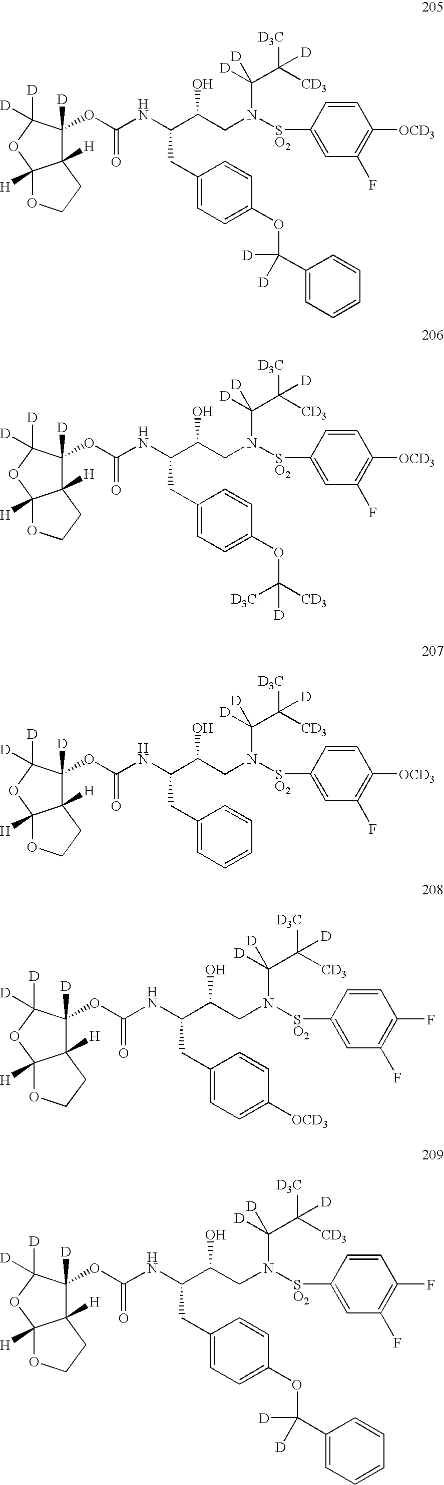

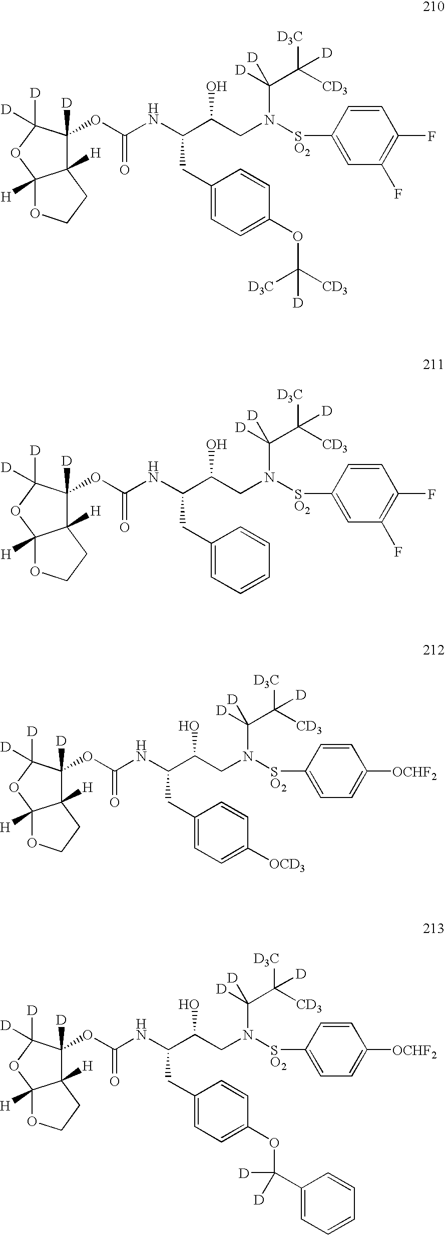

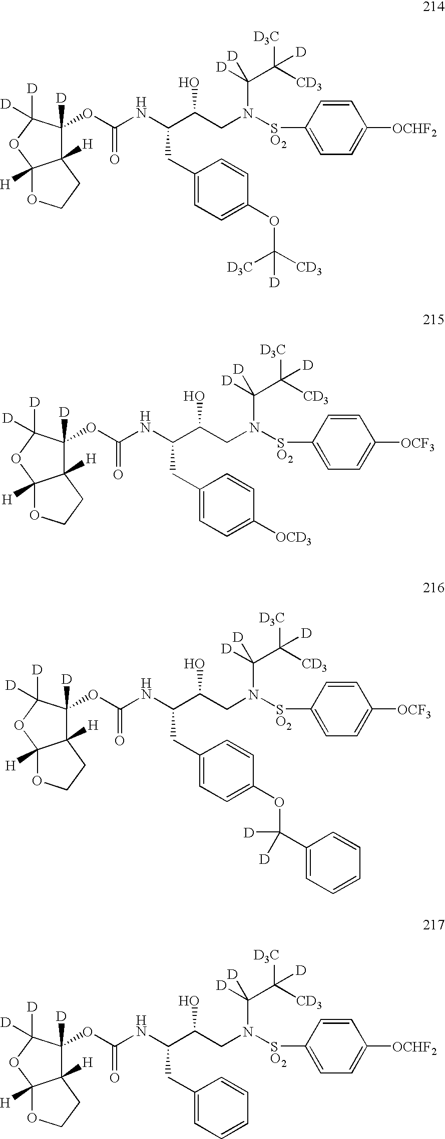

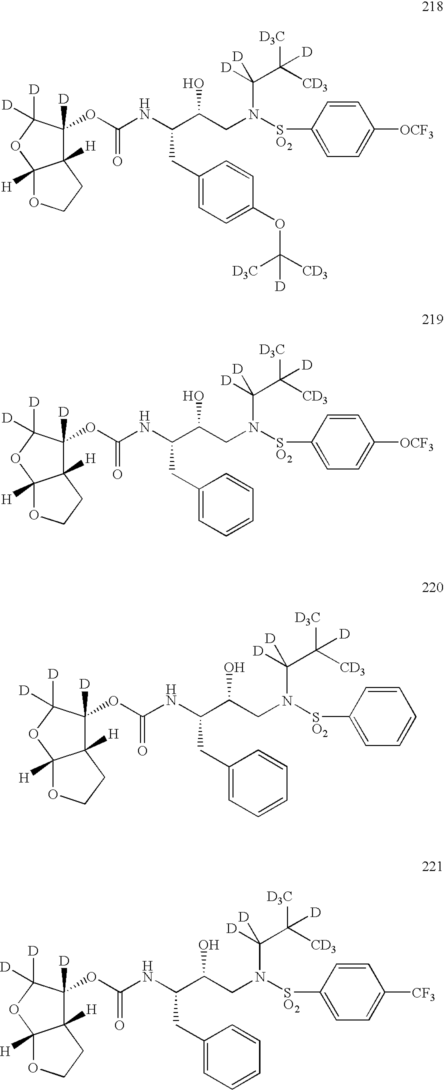

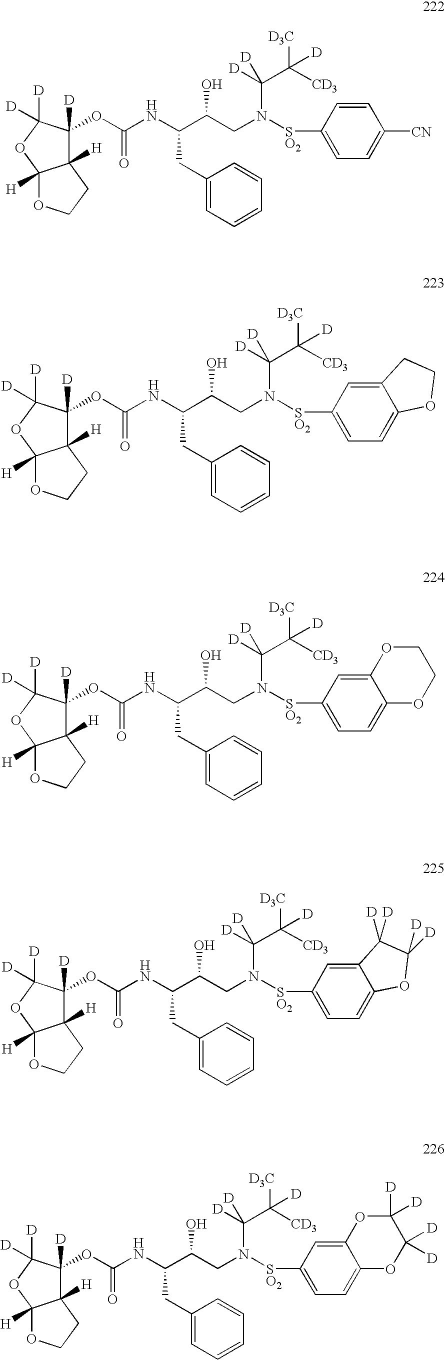

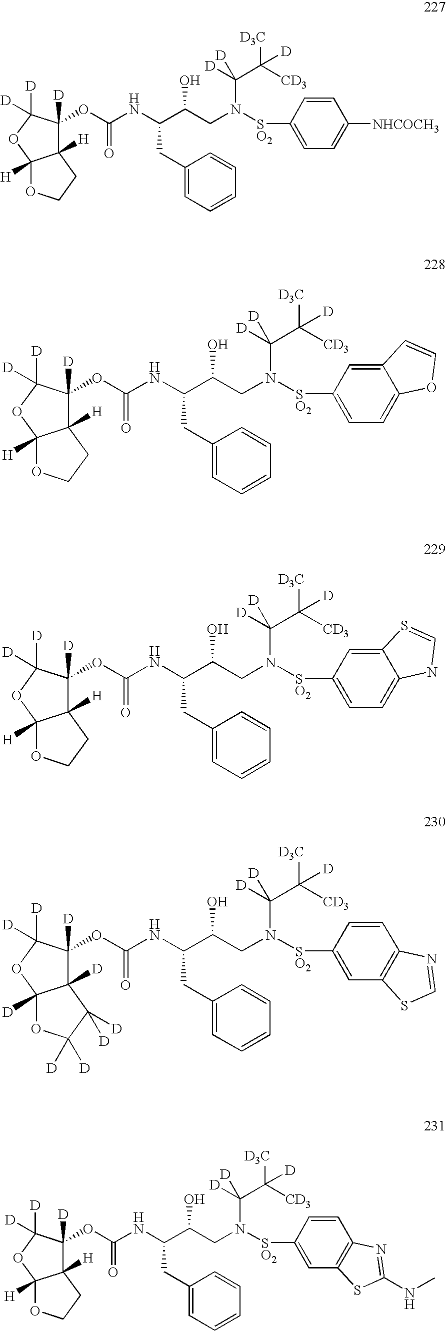

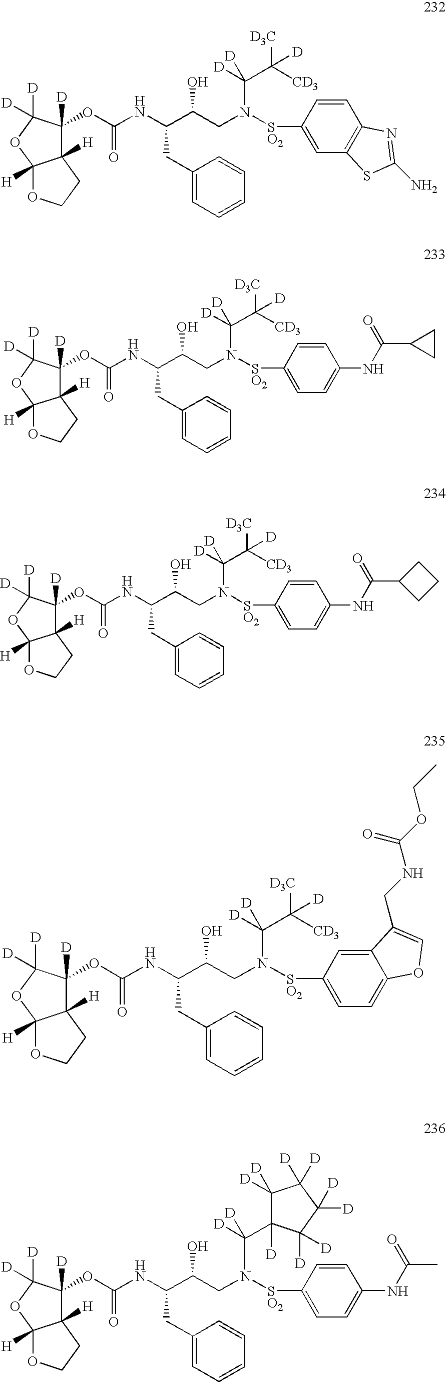

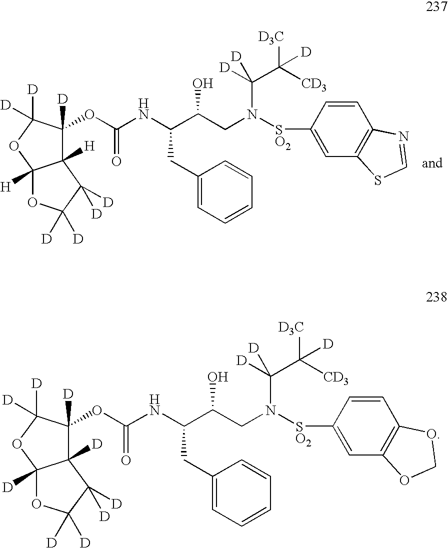

15. The compound of claim 1, wherein any atom not designated as deuterium is present at its natural isotopic abundance.

16. A pharmaceutical composition comprising a compound of claim 1, or a pharmaceutically acceptable salt thereof and a pharmaceutically acceptable carrier.

17. (canceled)

18. The composition of claim 16, additionally comprising a second therapeutic agent, wherein the second therapeutic agent is ritonavir.

19. A method of treating a disease or condition selected from HIV infection and malaria in a patient in need thereof comprising administering to the patient an effective amount of a composition of claim 16.

20. The method of claim 19, wherein the disease or condition is HIV infection.

21. (canceled)

22. (canceled)

23. The method of claim 20, further comprising administering to the patient in need thereof a second therapeutic agent useful in the treatment of HIV infection, wherein the second therapeutic agent is ritonavir.

24. The compound of claim 6, wherein R³ is —CD(CD₃)₂.
